(19) United States
(12) Patent Application Publication
(10) Pub. No.: US 2007/0161633 A1
(43) Pub. Date: Jul. 12, 2007

(54) 4-Bromo-5-(2-chloro-benzoylamino)-1h-pyrazole-3-carboxylic acid (1-aminocarbonyl)eth-1-yl) amide derivatives and related compounds as bradykinin b1 receptor antagonists for the treatment of inflammatory diseases

(75) Inventors: Jay S. Tung, Belmont, CA (US); Albert W. Garofalo, South San Francisco, CA (US); Michael A. Pleiss, Sunnyvale, CA (US); Jing Wu, Redwood City, CA (US); David W.G. Wone, Newark, CA (US); Ashley C. Guinn, Santa Monica, CA (US); Darren B. Dressen, San Mateo, CA (US); Jennifer Marugg, San Jose, CA (US); Martin Neitzel, Pacifica, CA (US)

Correspondence Address: FOLEY & LARDNER LLP, 1530 PAGE MILL ROAD, PALO ALTO, CA, 94304, US

(73) Assignee: Elan Pharmaceuticals, Inc., South San Francisco, CA (US)

(21) Appl. No.: 10/555,515

(22) PCT Filed: Apr. 30, 2004
(86) PCT No.: PCT/US04/13400

§ 371 Date: Aug. 25, 2006

Related U.S. Application Data
(60) Provisional application No. 60/467,695, filed on May 2, 2003.
(60) Provisional application No. 60/503,269, filed on Sep. 15, 2003.

(51) Int. Cl. A61K 31/5377; A61K 31/496; A61K 31/454; A61K 31/4152; C07D 413/02; C07D 403/02

(52) U.S. Cl. 514/235.5; 546/211; 544/140; 544/371; 514/254.05; 514/326; 514/406; 548/364.1; 548/371.7

(57) ABSTRACT

Disclosed are compounds of formulae (I) and (II) that are bradykinin B₁ receptor antagonists and are useful for treating diseases, or relieving adverse symptoms associated with disease conditions, in mammals mediated by bradykinin B₁ receptor. Certain of the compounds exhibit increased potency and are also expected to exhibit increased duration of action. Wherein Z is selected from O, S and NH; Q of formula (III) and the other substituents are as defined in claim 1.

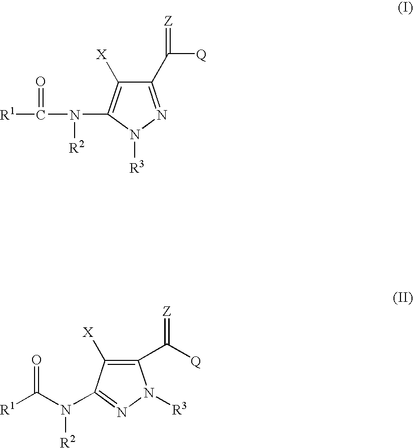

### CROSS-REFERENCE TO RELATED APPLICATIONS

This application claims the benefit of U.S. Provisional Application Ser. No. 60/467,695, filed on May 2, 2003 and U.S. Provisional Application Ser. No. 60/503,269, filed on Sep. 15, 2003, which applications are incorporated herein in their entirety.

### BACKGROUND OF THE INVENTION

### Field of the Invention

This invention is directed to certain 3-amido-5-substituted pyrazole derivatives and related compounds. These compounds are useful as bradykinin B₁ receptor antagonists to relieve adverse symptoms in mammals mediated, at least in part, by bradykinin B₁ receptor including pain, inflammation, septic shock, the scarring process, etc.

### REFERENCES

The following literature and patent publications are cited in this application as superscript numbers.
1 J. G. Menke, et al., J. Biol. Chem., 269(34):21583-21586 (1994).
2 J. F. Hess, Biochem. Human B₂ Receptor, Biophys. Res. Commun., 184:260-268 (1992).
3 R. M. Burch, et al., “Bradykinin Receptor Antagonists”, Med. Res. Reviews, 10(2):237-269 (1990).
4 Clark, W. G. “Kinins and the Peripheral Central Nervous Systems”, Handbook of Experimental Pharmacology, Vol. XXV: Bradykinin, Kallidin, and Kallikrein. Erdo, E. G. (Ed.), 311-322 (1979).
5 Ammons, W. S., et al., “Effects of Intracardiac Bradykinin on T₂-T₅ Medial Spinothalamic Cells”, American Journal of physiology, 249, R145-152 (1985).
6 Costello, A. H. et al., “Suppression of Carageenan-Induced Hyperalgesia, Hyperthermia and Edema by a Bradykinin Antagonist”, European Journal of Pharmacology, 171:259-263 (1989).
7 Laneuville, et al., “Bradykinin Analogue Blocks Bradykinin-induced Inhibition of a Spinal Nociceptive Reflex in the Rat”, European Journal of Pharmacology, 137:281-285 (1987).
8 Steranka, et al., “Antinociceptive Effects of Bradykinin Antagonists”, European Journal of Pharmacology, 136:261-262 (1987).
9 Steranka, et al., “Bradykinin as a Pain Mediator: Receptors are Localized to Sensory Neurons, and Antagonists have Analgesic Actions”, Neurobiology, 85:3245-3249 (1987).
10 Whalley, et al., in Naunyn Schmiederberg's Arch. Pharmacol., 336:652-655 (1987).
11 Back, et al., “Determination of Components of the Kallikrein-Kinin System in the Cerebrospinal Fluid of Patients with Various Diseases”, Res. Clin. Stud. Headaches, 3:219-226 (1972).
12 Ness, et al., “Visceral pain: a Review of Experimental Studies”, Pain, 41:167-234 (1990).
13 Aasen, et al., “Plasma kallikrein Activity and Prekallikrein Levels during Endotoxin Shock in Dogs”, Eur. Surg., 10:5062(1977).
14 Aasen, et al., “Plasma Kallikrein-Kinin System in Septicemia”, Arch. Surg., 118:343-346 (1983).
15 Katori, et al., “Evidence for the Involvement of a Plasma Kallikrein/Kinin System in the Immediate Hypotension Produced by Endotoxin in Anaesthetized Rats”, Br. J. Pharmacol., 98:1383-1391 (1989).
16 Marceau, et al., “Pharmacology of Kinins: Their Relevance to Tissue Injury and Inflammation”, Gen. Pharmacol., 14:209-229 (1982).
17 Weipert, et al., Brit J. Pharm., 94:282-284 (1988).
18 Haberland, “The Role of Kininogenases, Kinin Formation and Kininogenase Inhibitor in Post Traumatic Shock and Related Conditions”, Klinische Woochen-Schrift, 56:325-331 (1978).
19 Ellis, et al., “Inhibition of Bradykinin-and Kallikrein-Induced Cerebral Arteriolar Dilation by Specific Bradykinin Antagonist”, Stroke, 18:792-795 (1987).
20 Kamitani, et al., “Evidence for a Possible Role of the Brain Kallikrein-Kinin System in the Modulation of the Cerebral Circulation”, Circ. Res., 57:545-552 (1985).
21 Barnes, “Inflammatory Mediator Receptors and Asthma”, Am. Rev. Respir. Dis., 135:526-S31 (1987).
23 Fuller, et al., “Bradykinin-induced Bronchoconstriction in Humans”, Am. Rev. Respir. Dis., 135:176-180 (1987).
24 Jin, et al., “Inhibition of Bradykinin-Induced Bronchoconstriction in the Guinea-Pig by a Synthetic B₂ Receptor Antagonist”, Br. J. Pharmacol., 97:598-602 (1989).
25 Polosa, et al., “Contribution of Histamine and Prostanoids to Bronchoconstriction Provoked by Inhaled Bradykinin in Atopic Asthma”, Allergy, 45:174-182 (1990).
26 Baumgarten, et al., “Concentrations of Glandular Kallikrein in Human Nasal Secretions Increase During Experimentally Induced Allergic Rhinitis”, J. Immunology, 137:1323-1328 (1986).
27 Proud, et al., “Nasal Provocation with Bradykinin Induces Symptoms of Rhinitis and a Sore Throat”, Am. Rev. Respir Dis., 137:613-616 (1988).
28 Steward and Vavrek in “Chemistry of Peptide Bradykinin Antagonists” Basic and Chemical Research, R. M. Burch (Ed.), pages 51-96 (1991).
29 Seabrook, et al., Expression of B1 and B2 Bradykinin Receptor mRNA and Their Functional Roles in Sympathetic Ganglia and Sensory Dorsal Root Ganglia Neurons from Wild-type and B2 Receptor Knockout Mice, Neuropharmacology, 36(7):1009-17 (1997).
30 Elguero, et al., Nonconventional Analgesics: Bradykinin Antagonists, An. R. Acad. Farm., 63(1):173-90 (Spa) (1997).
31 McManus, U.S. Pat. No. 3,654,275, Quinoxalinecarboxamide Antiinflammatory Agents, issued Apr. 4, 1972.
32 Beyreuther, B.; et al., International Patent application publication number WO 03/007958 A1 filed on Jul. 4, 2002.
33 Marceau, “Kinin B₁ Receptors: A Review,” Immunopharmacology, 30:1-26 (1995).
34 Giese, et al., U.S. Pat. No. 5,916,908, issued Jun. 29, 1999.
35 Yoshida, et al., Japanese Patent Application Serial No. 49100080.
36 Oxford Dictionary of Biochemistry and Molecular Biology. Oxford University Press, 2001.

All of the above-identified publications are herein incorporated by reference in their entirety to the same extent as if each individual publication was specifically and individually incorporated by reference in its entirety.

### State of the Art

Bradykinin or Kinin-9 (BK) is a vasoactive nonapeptide, H-Arg¹-Pro²-Pro³-Gly⁴-Phe⁵-Ser⁶-Pro⁷-Phe⁸-Arg⁹ —OH (SEQ. ID. NO. 1), formed by the action of plasma kallikrein, which hydrolyses the sequence out of the plasma globulin kininogen. Plasma kallikrein circulates as an inactive zymogen, from which active kallikrein is released by Hageman factor. Tissue kallikrein appears to be located predominantly on the outer surface of epithelial cell membranes at sites thought to be involved in transcellular electrolyte transport.

Glandular kallikrein cleaves kininogen one residue earlier to give the decapeptide Lys-bradykinin (kallidin, Lys-BK) (SEQ. ID. NO. 2). Met-Lys-bradykinin (SEQ. ID. NO. 3) is also formed, perhaps by the action of leukocyte kallikrein. Pharmacologically important analogues include des-Arg⁹ or BK₁₋₈ (Amino Acids 1-8 of SEQ. ID. NO. 1) and Ile-Ser-bradykinin (or T-kinin) (SEQ. ID. NO. 4), [Hyp³]bradykinin (SEQ. ID. NO. 5), and [Hyp⁴]bradykinin (SEQ. ID. NO. 6).³⁶

Bradykinin (BK) (SEQ. ID. NO. 1) is known to be one of the most potent naturally occurring stimulators of C-fiber afferents mediating pain. It also is a powerful blood-vessel dilator, increasing vascular permeability and causing a fall in blood pressure, edema-producing agent, and stimulator of various vascular and non-vascular smooth muscles in tissues such as uterus, gut and bronchiole. Bradykinin (SEQ. ID. NO. 1) is formed in a variety of inflammatory conditions and in experimental anaphylactic shock. The kinin/kininogen activation pathway has also been described as playing a pivotal role in a variety of physiologic and pathophysiologic processes, being one of the first systems to be activated in the inflammatory response and one of the most potent simulators of: (i) phospholipase A₂ and, hence, the generation of prostaglandins and leukotriene; and (ii) phospholipase C and thus, the release of inositol phosphates and diacylgylcerol. These effects are mediated predominantly via activation of BK receptors of the BK₂ type.

Bradykinin receptor is any membrane protein that binds bradykinin (BK) (SEQ. ID. NO. 1) and mediates its intracellular effects. Two types of receptors are recognized: B₁, on which order of potency is des-Arg⁹-bradykinin (BK₁₋₈) (Amino Acids 1-8 of SEQ. ID. NO. 1)=kallidin (Lys-BK) (SEQ. ID. NO. 2)>BK (SEQ. ID. NO. 1); and B₂, with order of potency kallidin (SEQ. ID. NO. 2)>BK (SEQ. ID. NO. 1)>>BK₁₋₈ (Amino Acids 1-8 of SEQ. ID. NO. 1). Hence, BK₁₋₈ (Amino Acids 1-8 of SEQ. ID. NO. 1) is a powerful discriminator.³⁶ B₁ receptors are considerably less common than B₂ receptors, which are present in most tissues. The rat B₂ receptor is a seven-transmembrane-domain protein which has been shown on activation to stimulate phosphoinositide turnover. The B₁ subtype is induced by inflammatory processes.³³ The distribution of receptor B₁ is very limited since this receptor is only expressed during states of inflammation. BK receptors have been cloned for different species, notably the human B₁ receptor (see J. G. Menke et al.¹, and human B2 receptor J. F. Hess²). Examples: B₁, database code BRB1_HUMAN, 353 amino acids (40.00 kDa); B₂, database code BRB2_HUMAN, 364 amino acids (41.44 kDa).³⁶

Two major kinin precursor proteins, high molecular weight and low molecular weight kininogen are synthesized in the liver, circulate in plasma, and are found in secretions such as urine and nasal fluid. High molecular weight kininogen is cleaved by plasma kallikrein, yielding BK (SEQ. ID. NO. 1), or by tissue kallikrein, yielding kallidin. Low molecular weight kininogen, however, is a substrate only for tissue kallikrein. In addition, some conversion of kallidin to BK (SEQ. ID. NO. 1) may occur inasmuch as the amino terminal lysine residue of kallidin (SEQ. ID. NO. 2) is removed by plasma aminopeptidases. Plasma half-lives for kinins are approximately 15 seconds, with a single passage through the pulmonary vascular bed resulting in 80-90% destruction. The principle catabolic enzyme in vascular beds is the dipeptidyl carboxypeptidase kininase II or angiotensin-converting enzyme (ACE). A slower acting enzyme, kininase I, or carboxypeptidase N, which removes the carboxyl terminal Arg, circulates in plasma in great abundance. This suggests that it may be the more important catabolic enzyme physiologically. Des-Arg⁹-bradykinin (Amino Acids 1-8 of SEQ. ID. NO. 1) as well as des-Arg¹⁰-kallidin (Amino Acids 1-9 of SEQ. ID. NO. 2) formed by kininase I acting on BK (SEQ. ID. NO. 1) or kallidin (SEQ. ID. NO. 2), respectively, are active BK₁ receptor agonists, but are relatively inactive at the more abundant BK₂ receptor at which both BK (SEQ. ID. NO. 1) and kallidin (SEQ. ID. NO. 2) are potent agonists.

Direct application of bradykinin (SEQ. ID. NO. 1) to denuded skin or intra-arterial or visceral injection results in the sensation,of pain in mammals including humans. Kinin-like materials have been isolated from inflammatory sites produced by a variety of stimuli. In addition, bradykinin receptors have been localized to nociceptive peripheral nerve pathways and BK (SEQ. ID. NO. 1) has been demonstrated to stimulate central fibers mediating pain sensation. Bradykinin (SEQ. ID. NO. 1) has also been shown to be capable of causing hyperalgesia in animal models of pain. See, Burch, et al,³ and Clark, W. G.⁴

These observations have led to considerable attention being focused on the use of BK antagonists as analgesics. A number of studies have demonstrated that BK antagonists are capable of blocking or ameliorating both pain as well as hyperalgesia in mammals including humans. See, Ammons, W. S., et al.⁵, Clark, W. G.⁴, Costello, A. H., et al.⁶, Laneuville, et al.⁷, Steranka, et al.⁸ and Steranka, et al.⁹

Currently accepted therapeutic approaches to analgesia have significant limitations. While mild to moderate pain can be alleviated with the use of non-steroidal anti-inflammatory drugs and other mild analgesics, severe pain such as that accompanying surgical procedures, bums and severe trauma requires the use of narcotic analgesics. These drugs carry the limitations of abuse potential, physical and psychological dependence, altered mental status and respiratory depression which significantly limit their usefulness.

Prior efforts in the field of BK antagonists indicate that such antagonists can be useful in a variety of roles. These include use in the treatment of burns, perioperative pain, migraine and other forms of pain, shock, central nervous system injury, asthma, rhinitis, premature labor, inflammatory arthritis, inflammatory bowel disease, neuropathic pain, etc. For example, Whalley, et al.¹⁰ has demonstrated that BK antagonists are capable of blocking BK-induced pain in a human blister base model. This suggests that topical application of such antagonists would be capable of inhibiting pain in burned skin, e.g., in severely burned patients that require large doses of narcotics over long periods of time and for the local treatment of relatively minor burns or other forms of local skin injury.

The management of perioperative pain requires the use of adequate doses of narcotic analgesics to alleviate pain while not inducing excessive respiratory depression. Post-operative narcotic-induced hypoventilation predisposes patients to collapse of segments of the lungs, a common cause of post-operative fever, and frequently delays discontinuation of mechanical ventilation. The availability of a potent non-narcotic parenteral analgesic could be a significant addition to the treatment of perioperative pain. While no currently available BK antagonist has the appropriate pharmacodynamic profile to be used for the management of chronic pain, frequent dosing and continuous infusions are already commonly used by anesthesiologists and surgeons in the management of perioperative pain.

Several lines of evidence suggest that the kallikrein/kinin pathway may be involved in the initiation or amplification of vascular reactivity and sterile inflammation in migraine. (See, Back, et al.11). Because of the limited success of both prophylactic and non-narcotic therapeutic regimens for migraine as well as the potential for narcotic dependence in these patients, the use of BK antagonists offers a highly desirable alternative approach to the therapy of migraine.

Bradykinin (SEQ. ID. NO. 1) is produced during tissue injury and can be found in coronary sinus blood after experimental occlusion of the coronary arteries. In addition, when directly injected into the peritoneal cavity, BK (SEQ. ID. NO. 1) produces a visceral type of pain. (See, Ness, et al. 12). While multiple other mediators are also clearly involved in the production of pain and hyperalgesia in settings other than those described above, it is also believed that antagonists of BK (SEQ. ID. NO. 1) have a place in the alleviation of such forms of pain as well.

Shock related to bacterial infections is a major health problem. It is estimated that 400,000 cases of bacterial sepsis occur in the United States yearly; of those, 200,000 progress to shock, and 50% of these patients die. Current therapy is supportive, with some suggestion in recent studies that monoclonal antibodies to Gram-negative endotoxin may have a positive effect on disease outcome. Mortality is still high, even in the face of this specific therapy, and a significant percentage of patients with sepsis are infected with Gram-positive organisms that would not be amenable to anti-endotoxin therapy.

Multiple studies have suggested a role for the kallikrein/kinin system in the production of shock associated with endotoxin. See, Aasen, et al.¹³, Aasen, et al.¹⁴, Katori, et al.¹⁵ and Marceau, et al.¹⁶ Recent studies using newly available BK antagonists have demonstrated in animal models that these compounds can profoundly affect the progress of endotoxic shock. (See, Weipert, et al.¹⁷). Less data is available regarding the role of BK (SEQ. ID. NO. 1) and other mediators in the production of septic shock due to Gram-positive organisms. However, it appears likely that similar mechanisms are involved. Shock secondary to trauma, while frequently due to blood loss, is also accompanied by activation of the kallikrein/kinin system. (See, Haberland.¹⁸)

Numerous studies have also demonstrated significant levels of activity of the kallikrein/kinin system in the brain. Both kallikrein and BK (SEQ. ID. NO. 1) dilate cerebral vessels in animal models of central nervous system (CNS) injury. (See Ellis, et al.¹⁹ and Kamitani, et al.²⁰). BK antagonists have also been shown to reduce cerebral edema in animals after brain trauma. Based on the above, it is believed that BK antagonists should be useful in the management of stroke and head trauma.

Other studies have demonstrated that BK receptors are present in the lung, that BK (SEQ. ID. NO. 1) can cause bronchoconstriction in both animals and man and that a heightened sensitivity to the bronchoconstrictive effect of BK (SEQ. ID. NO. 1) is present in asthmatics. Some studies have been able to demonstrate inhibition of both BK (SEQ. ID. NO. 1) and allergen-induced bronchoconstriction in animal models using BK antagonists. These studies indicate a potential role for the use of BK antagonists as clinical agents in the treatment of asthma (See Barnes²¹, Burch, et al.³, Fuller, et al.²³, Jin, et al.²⁴ and Polosa, et al.²⁵.) BK (SEQ. ID. NO. 1) has also been implicated in the production of histamine and prostanoids to bronchoconstriction provoked by inhaled bradykinin in atopic asthma.²⁵ Bradykinin (SEQ. ID. NO. 1) has also been implicated in the production of symptoms in both allergic and viral rhinitis. These studies include the demonstration of both kallikrein and BK (SEQ. ID. NO. 1) in nasal lavage fluids and that levels of these substances correlate well with symptoms of rhinitis. (See, Baumgarten, et al.²⁶, Jin, et al.²⁴, and Proud, et al.²⁷)

In addition, studies have demonstrated that BK (SEQ. ID. NO. 1) itself can cause symptoms of rhinitis. Stewart and Vavrek²⁸ discuss peptide BK antagonists and their possible use against effects of BK (SEQ. ID. NO. 1). A great deal of research effort has been expended towards developing such antagonists with improved properties. However, notwithstanding extensive efforts to find such improved BK antagonists, there remains a need for additional and more effective BK antagonists. Two of the major problems with presently available BK antagonists are their low levels of potency and their extremely short durations of activity. Thus there is a special need for BK antagonists having increased potency and for duration of action.

Two generations of peptidic antagonists of the B2 receptor have been developed. The second generation has compounds two orders of magnitude more potent as analgesics than first generation compounds and the most important derivative was icatibant. The first non-peptidic antagonist of the B2 receptor, described in 1993, has two phosphonium cations separated by a modified amino acid. Many derivatives of this di-cationic compound have been prepared. Another non-peptidic compound antagonist of B2 is the natural product Martinelline. See Elguero, et al.³⁰ See also Seabrook.²⁹

U.S. Pat. No. 3,654,275³¹ teaches that certain 1,2,3,4-tetrahydro-1-acyl-3-oxo-2-quinoxalinecarboxamides have anti-inflammatory activity.

International Patent Application WO 03/007958 filed on Jul. 2, 2002 and published on Jan. 30, 2003 discloses tetrahydroquinoxalines acting as bradykinin antagonists.³²

U.S. Pat. No. 5,916,908³⁴ teaches the use of 3,5-disubstituted pyrazoles or 3,4,5-trisubstituted pyrazoles as kinase inhibitors.

Japanese Patent Application Serial No. 49100080³⁵ teaches 2-aminopyrazoles as anti-inflammatory agents.

Currently there is no marketed therapeutic agent for the inhibition of bradykinin B₁ receptor. In view of the above, compounds which are bradykinin B₁ receptor antagonists would be particularly advantageous in treating those diseases mediated by bradykinin B₁ receptor.

### SUMMARY OF THE INVENTION

This invention is directed, in part, to compounds that are bradykinin B₁ receptor antagonist. It is also directed to compounds that are useful for treating diseases or relieving adverse symptoms associated with disease conditions in mammals, where the disease is mediated at least in part by bradykinin B₁ receptor. For example, inhibition of the bradykinin B₁ receptor is useful for the moderation of pain, inflammation, septic shock, the scarring process, etc. These compounds are preferably selective for antagonism of the B₁ receptor over the B₂ receptor. Certain of the compounds exhibit increased potency and are expected to also exhibit an increased duration of action.

In one embodiment, this invention provides compounds of Formula (I) and/or Formula (II): 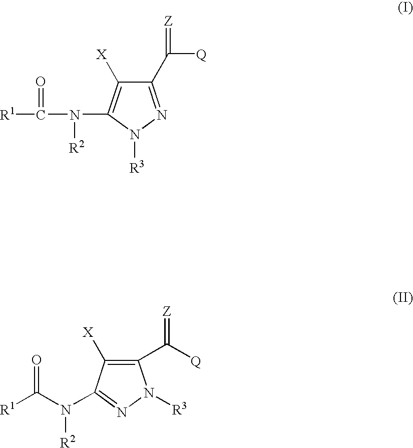
wherein

Z is selected from O, S and NH;

Q is

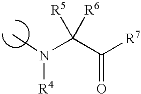

R¹ is selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic and substituted heterocyclic;

R² and R⁴ are independently selected from the group consisting of hydrogen, alkyl and substituted alkyl;

R³ is selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic and substituted heterocyclic;

R⁵ and R⁶ are independently selected from hydrogen, and the side chain of a natural or unnatural amino acid, wherein R⁵ and R⁶ may optionally be linked together to form a cycloalkyl or substituted cycloalkyl;

R⁷ is selected from the group consisting of —NR^{b}R^{c} and —OR^{b} wherein R^{b} and R^{c} are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic, or R^{b} and R^{c} are joined together with the nitrogen atom pendent thereto to form a heterocyclic or substituted heterocyclic group;

X is selected from the group consisting of hydrogen, alkyl, substituted alkyl, alkoxy, substituted alkoxy, aryl, substituted aryl, carboxyl, carboxyl esters, cyano, halo, heteroaryl, substituted heteroaryl, hydroxy, nitro, amino, substituted amino, acylamino, and aminoacyl;

or pharmaceutically acceptable salts, prodrugs or isomers thereof.

In Formula (I) or Formula (II), Z is preferably O.

In Formula (I) or Formula (II), preferred R¹ groups include aryl and substituted aryl groups. Preferred aryl groups include phenyl, naphth-2-yl, naphth-1-yl; and the like. Preferred substituted aryl groups include monosubstituted phenyls, monosubstituted naphthyls, disubstituted phenyls, trisubstituted phenyls, and the like.

Particularly preferred R¹ groups include, by way of example only, 5-dimethylaminonaphth-1-yl, 2-chlorophenyl, 2-fluorophenyl, 2-bromophenyl, 2-hydroxyphenyl, 2-nitrophenyl, 2-methylphenyl, 2-methoxyphenyl, 2-phenoxyphenyl, 2-trifluoromethylphenyl, 4-fluorophenyl, 4-chlorophenyl, 4-bromophenyl, 4-nitrophenyl, 4-methylphenyl, 4-hydroxyphenyl, 4-methoxyphenyl, 4-ethoxyphenyl, 4-butoxyphenyl, 4-isopropylphenyl, 4-phenoxyphenyl, 4-trifluoromethylphenyl, 4-hydroxymethylphenyl, 3-methoxyphenyl, 3-hydroxyphenyl, 3-nitrophenyl, 3-fluorophenyl, 3-chlorophenyl, 3-bromophenyl, 3-phenoxyphenyl, 3-thiomethoxyphenyl, 3-methylphenyl, 3-trifluoromethylphenyl, 2,3-dichlorophenyl, 2,3-difluorophenyl, 2,4-dichlorophenyl, 2,5-dimethoxyphenyl, 3,4-dichlorophenyl, 3,4-difluorophenyl, 3,4-methylenedioxyphenyl, 3,4-dimethoxy-phenyl, 3,5-difluorophenyl, 3,5-dichlorophenyl, 3,5-di-(trifluoromethyl)phenyl, 3,5-dimethoxyphenyl, 2,4-dichlorophenyl, 2,4-difluorophenyl, 2,6-difluorophenyl, 3,4,5-trifluorophenyl, 3,4,5-trimethoxyphenyl, 3,4,5-tri-(trifluoromethyl)phenyl, 2,4,6-trifluorophenyl, 2,4,6-trimethylphenyl, 2,4,6-tri-(trifluoromethyl)phenyl, 2,3,5-trifluorophenyl, 2,4,5-trifluorophenyl, 2,5-difluorophenyl, 2-fluoro-3-trifluoromethylphenyl, 4-fluoro-2-trifluoromethylphenyl, 2-fluoro-4-trifluoromethylphenyl, 4-benzyloxyphenyl, 2-chloro-6-fluorophenyl, 2,3,4,5,6-pentafluorophenyl, 2,5-dimethylphenyl, 4-phenylphenyl, 2-fluoro-3-trifluoromethylphenyl, 2-(quinolin-8-yl) thiomethyl)phenyl, 2-((3-methylphen-1-ylthio)methyl)phenyl, and the like.

Preferred R¹ alkyl, substituted alkyl, alkenyl and cycloalkyl groups in Formula (I) or Formula (II) include, by way of example, isopropyl, n-propyl, n-butyl, isobutyl, sec-butyl, t-butyl, —CH₂CH═CH₂, —CH₂CH═CH(CH₂)₄CH₃, cyclopropyl, cyclobutyl, cyclohexyl, cyclopentyl, —CH₂-cyclopropyl, —CH₂-cyclobutyl, —CH₂-cyclohexyl, —CH₂-cyclopentyl, —CH₂CH₂-cyclopropyl, —CH₂CH₂-cyclobutyl, —CH₂CH₂-cyclohexyl, —CH₂CH₂-cyclopentyl, benzyl, 2-phenyleth-1-yl, 3-phenyl-n-prop-1-yl, and the like.

Preferred R¹ heteroaryls and substituted heteroaryls in Formula (I) or Formula (II) include, by way of example, pyrid-2-yl, pyrid-3-yl, pyrid-4-yl, fluoropyridyls (including 5-fluoropyrid-3-yl), chloropyridyls (including 5-chloropyrid-3-yl), thiophen-2-yl, thiophen-3-yl, benzothiazol-4-yl, 2-phenylbenzoxazol-5-yl, furan-2-yl, benzofuran-2-yl, thionaphthen-2-yl, 2-chlorothiophen-5-yl, 3-methylisoxazol-5-yl, 2-(thiophenyl)thiophen-5-yl, 6-methoxythionaphthen-2-yl, 3-phenyl-1,2,4-thiooxadiazol-5-yl, 2-phenyloxazol-4-yl, 5-chloro-1,3-dimethylpyrazol-4-yl; 2-methoxycarbonyl-thiophen-3-yl; 2,3-dimethylimidazol-5-yl; 2-methylcarbonylamino-4-methyl-thiazol-5-yl; quinolin-8-yl; thiophen-2-yl; 1-methylimidiazol-4-yl; 3,5-dimethylisoxazol-4-yl; and the like.

Especially preferred R¹ groups are 2-chlorophenyl, 2-fluorophenyl, 2-(quinolin-8-yl) thiomethyl)phen-1-yl and 2-((3-methylphen-1-ylthio)methyl)phen-1-yl.

R¹ may be also be sulfonated aminoalkyl such as Formula (V) below, wherein R²¹ is hydrogen or alkyl, and R²⁰ is an amino acid side chain or where R²⁰ and R²¹ and the atoms to which they are attached form a heterocyclic or heteroaryl group of from 4 to 12 ring atoms, and R²² is alkyl, substituted alkyl, aryl or substituted aryl.

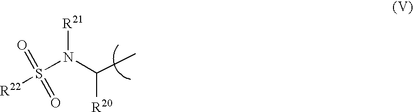

In one embodiment, R¹ is N-(4-methylbenzenesulfonyl)pyrrol-2-yl, N-(4-chloro-2,5-dimethylbenzenesulfonyl)pyrrol-2-yl, N-(napthylsulfonyl)pyrrol-2-yl, N-(bezylsulfonyl)pyrrol-2-yl; N-(4-chloro-2,5-dimethylbenzenesulfonyl)azetidin-2-yl, N-(4-chloro-2,5-dimethylbenzenesulfonyl)piperidin-2-yl, 1-(4-chloro-2,5-dimethylbenzenesulfonyl)-1,2,3,4-tetrahydroisoquinolin-2-yl, N-(4-chloro-2,5-dimethylbenzenesulfonyl)-N-methyl-aminomethyl; and 1-[N-(4-chloro-2,5-dimethylbenzenesulfonyl)amino]eth-1-yl; and the like.

R²² is preferably selected from the group consisting of phenyl, 4-methylphenyl, 2,5-dimethylphenyl, 4-chlorophenyl, 2,5-dimethyl-4-chlorophenyl, benzyl, naphthyl, 1,2,3,4-tetrahydroisoquinoline, and the like.

R²⁰ is preferably hydrogen.

R²¹ is preferably hydrogen, methyl, or ethyl.

Preferably R²⁰ and R²¹ are joined to form a heterocyclic group, such as azetidinyl, pyrrolidinyl, piperidinyl, 1,2,3,4-tetrahydroisoquinolinyl, and the like.

Preferred R² and R⁴ groups include hydrogen, methyl, ethyl, isopropyl, 2-methoxyeth-1-yl, pyrid-3-ylmethyl, benzyl, t-butoxycarbonyl-methyl and the like.

Particularly preferred R² and R⁴ groups are hydrogen.

Preferably R⁵ is hydrogen and R⁶ and is an amino acid sided chain.

Preferably the R⁵ and R⁶ amino acid side chain groups in Formula (I) or Formula (II) are independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic. Particularly preferred amino acid side chain groups in Formula (I) or (II) above include, by way of example only, hydrogen, methyl, ethyl, iso-propyl, n-propyl, n-butyl, isobutyl, sec-butyl, t-butyl, —CH₂CH₂CH₂NHC(═NH)NH₂, —CH₂C(O)NH₂, —CH₂C(O)OH, —CH₂SH, —CH₂CH₂C(O)OH, —CH₂CH₂C(O)NH₂, imidazole-5-ylmethyl, —CH₂CH₂CH₂CH₂NH₂, —CH₂CH₂SCH₃, hydroxymethyl, 1-hydroxyethyl, phenyl, 4-hydroxyphenyl, benzyl, 4-hydroxybenzyl, 2-phenylethyl, 3-phenyl-n-propyl, 1H-indol-3-ylmethyl, —CH₂CH═CH₂, —CH₂CH═CH(CH₂)₄CH₃, cyclopropyl, cyclobutyl, cyclohexyl, cyclopentyl, cyclohex-1-enyl, —CH₂-cyclopropyl, —CH₂-cyclobutyl, —CH₂-cyclohexyl, —CH₂-cyclopentyl, —CH₂CH₂-cyclopropyl, —CH₂CH₂-cyclobutyl, —CH₂CH₂-cyclohexyl, —CH₂CH₂-cyclopentyl, and the like.

In one embodiment, R⁷ is —OR^{b} where R^{b} is preferably selected from the group consisting of hydrogen, methyl, ethyl, isopropyl, benzyl, phenyl and the like.

In another embodiment, R⁷ is —NR^{a}R^{b} where R^{a} and R^{b} are independently selected from the group consisting of hydrogen, methyl, ethyl, isopropyl, phenyl, benzyl, 2-dimethylcarbamoylphenyl, and the like.

In still another embodiment, R⁷ is —NR^{a}R^{b} and R^{a} and R^{b} together with the nitrogen atom pendent thereto form are a piperidinyl, morpholino, thiomorpholino, pyrazinyl, 4-methylpyrazin-1-yl, and the like.

In one embodiment, R³ is selected from hydrogen, methyl, ethyl, isopropyl, 2-methoxyeth-1-yl, pyrid-3-ylmethyl, benzyl, t-butoxycarbonyl-methyl and the like. Particularly preferred R³ groups include hydrogen, C₁₋₄alkyl, optionally substituted monocyclic aryl, and optionally substituted monocyclic heteroaryl. Most preferred R³ groups are hydrogen, methyl and phenyl.

In one preferred embodiment, Q is an α-amino radical derived from the following natural and unnatural amino acids and their derivatives:
2-amino-N,N-dimethylpropanamide;
alaninamide;
alanine methyl ester;
(1-diethylcarbamoyl-ethyl)-amine;
[(2-dimethylcarbamoylphenylcarbamoyl)-methyl]amine;
(1-dimethylcarbamoyl-propyl)-amine;
[1-(ethyl-methyl-carbamoyl)-ethyl]-amine;
leucine methyl ester;
(1-methyl-2-morpholin-4-yl-2-oxo-ethyl)-amine;
[2-(4-methyl-piperazin-1-yl)-2-oxo-ethyl]-amine;
(1-methyl-2-oxo-2-piperidin-1-yl-ethyl)-amine;
norleucine methyl ester;
(2-oxo-1-phenyl-2-piperidin-1-yl-ethyl)-amine;
phenylalanine methyl ester;
2-phenylglycine methyl ester;
2-phenylglycine methylamide
[3-phenyl-1-(piperidine-1-carbonyl)-propyl]-amine;
tryptophan methyl ester;
2-phenylglycine;
2-phenylglycine methylamide;
2-amino-N-methyl-N-phenyl-acetamide;
tyrosine methyl ester;
2-phenylglycine amide;
2-amino-3-methyl-butyramide;
2-(p-hydroxyphenyl)glycine methyl ester;
1-(2-Amino-1-oxopropyl)piperidine;
4-Aza-DL-leucine;
Alanyl-L-proline;
beta-Alaninamide;
beta-Alanine t-butyl ester;
p-hydroxyphenylglycine methyl ester,
L-phenylglycine;
Phenylglycine amide;
Tyrosine methyl ester or
Valine amide.

In another preferred embodiment, Q is selected from the group consisting of ((1-methyl-piperizin-4-ylcarbonyl)methyl)amino; ((N-methyl-N-phenyl-aminocarbonyl)methyl)amino; (1-(R or S)-1-(methoxycarbonyl)-1-(4-hydroxyphen-1-yl)methyl)amino; (1-(R or S)-1-(methoxycarbonyl)-2-(4-hydroxyphen-1-yl)eth-1-yl)amino; (1-(R or S)-1-(methoxycarbonyl)-3-(methyl)but-1-yl)amino; (1-(R or S)-1-(methoxycarbonyl)eth-1-yl)amino; (1-(R or S)-1-(N-morpholinocarbonyl)eth-1-yl)amino; (1-(R)-1-(aminocarbonyl)-2-methylprop-1-yl)amino; (1-(R)-1-(methoxycarbonyl)-2-(1H-indol-3-yl)eth-1-yl)amino; (1-(R)-1-(N,N-dimethylaminocarbonyl)eth-1-yl)amino; (1-(R)-1-(N,N-diethylaminocarbonyl)eth-1-yl)amino; (1-(R)-1-(N,N-dimethylaminocarbonyl)prop-1-yl)amino; (1-(R)-1-(N-ethyl-N-methylaminocarbonyl)eth-1-yl)amino; (1-(R)-1-(N-morpholinocarbonyl)eth-1-yl)amino; (1-(R)-1-(phenyl)-1-(methoxycarbonyl)methyl)amino; (1-(R)-1-(phenyl)-1-(piperidin-1-ylcarbonyl)methyl)amino; (1-(R)-1-(piperidin-1-ylcarbonyl)-3-(phenyl)prop-1-yl)amino; (1-(R)-1-(piperidin-1-ylcarbonyl)eth-1-yl)amino; (1-(S)-1-(aminocarbonyl)eth-1-yl)amino; (1-(S)-1-(methoxycarbonyl)pent-1-yl)amino; (1-(S)-1-(N,N-dimethylaminocarbonyl)eth-1-yl)amino; (1-(S)-1-(phenyl)-1-(aminocarbonyl)methyl)amino; (1-(S)-1-(phenyl)-1-(carboxy)methylamino; (1-(S)-1-(phenyl)-1-(methoxycarbonyl)methyl)amino; (1-(S)-1-(phenyl)-1-(methylaminocarbonyl)methyl)amino; (1-(S)-1-phenyl)-1-(piperidin-1-ylcarbonyl)methyl)amino; (1-(S)-1-(piperidin-1-ylcarbonyl)eth-1-yl)amino; (1-(S)-2-(phenyl)-1-(methoxycarbonyl)eth-1-yl)amino; and [(2-(dimethylaminocarbonyl)phen-1-ylaminocarbonyl)methyl]amino.

In still another preferred embodiment, the —CR⁵R⁶C(O)R⁷ moiety of Q is a radical selected from the following groups:
(1-(R)-1-(N,N-dimethylaminocarbonyl)eth-1-yl)amino
(1-(S)-1-(N,N-dimethylaminocarbonyl)eth-1-yl)amino
(1-(S)-1-(phenyl)-1-(methoxycarbonyl)methylamino
(1-(S)-1-(phenyl)-1-(carboxy)methylamino
((1-(S)-2-(phenyl)-1-(methoxycarbonyl)eth-1-yl)amino
(1-(R)-1-(phenyl)-1-(methoxycarbonyl)methyl)amino
(1-(S)-1-(methoxycarbonyl)pent-1-yl)amino
(1-(S)-1-(phenyl)-1-(methylaminocarbonyl)methyl)amino
(1-(S)-1-(piperidin-1-ylcarbonyl)eth-1-yl)amino
(1-(N-morpholinocarbonyl)eth-1-yl)amino
(1-(R)-1-(piperidin-1-ylcarbonyl)eth-1-yl)amino
(1-(S)-1-(phenyl)-1-(piperidin-1-ylcarbonyl)methyl)amino
(1-(R)-1-(phenyl)-1-(piperidin-1-ylcarbonyl)methyl)amino
(1-(R)-1-(N,N-dimethylaminocarbonyl)prop-1-yl)amino
(1-(R)-1-(N-ethyl-N-methylaminocarbonyl)eth-1-yl)amino
(1-(R)-1-(N,N-diethylaminocarbonyl)eth-1-yl)amino
((N-methyl-N-phenyl-amino)carbonylmethyl)amino
((1-methyl-piperizin-4-ylcarbonyl)methyl)amino
(1-(R)-1-(piperidin-1-ylcarbonyl)-3-(phenyl)prop-1-yl)amino
(1-(methoxycarbonyl)-2-(4-hydroxyphen-1-yl)eth-1-yl)amino
(1-(methoxycarbonyl)-1-(4-hydroxyphen-1-yl)methyl)amino
(1-(R)-1-(aminocarbonyl)-2-(methylprop-1-yl)amino
1-(S)-1-(aminocarbonyl)eth-1-yl)amino
1-(S)-1-phenyl-1-(aminocarbonyl)methyl)amino
(1-(R)-1-(methoxycarbonyl)-2-(1H-indol-3-yl)eth-1-yl)amino
(1-(methoxycarbonyl)-3-(methyl)but-1-yl)amino
(1-(methoxycarbonyl)eth-1-yl)amino
[(2-(dimethylaminocarbonyl)phen-1-ylaminocarbonyl)methyl]amino
2-[(4-amidino)phenyl]-1-(R)-(pyrrolidin-N-ylcarbonyl)eth-1-yl;
1-(S)-carboxamide-2-(indol-3-yl)eth-1-yl;
carboxamidemethyl;
1-carboxamide-2-(S)-methyl-but-1-yl;
1-(R)-carboxamide-2-(phenyl)eth-1-yl;
2-(4-cyanophenyl)-1-(R)-(pyrrolidin-N-ylcarbonyl)eth-1-y;
2-(4-cyanophenyl)-1-(S)-(pyrrolidin-N-ylcarbonyl)eth-1-yl;
2-(N-cyclopropylpiperidin-4-yl)-1-(R)-(pyrrolidin-N-ylcarbonyl)eth-1-yl;
1-(R)-1,3-di(benzyloxycarbonyl)prop-1-yl;
1-(S)-1,3-dicarboxamideprop-1-yl;
1-(S)-ethoxycarbonyleth-1-yl;
1-(R)-(1-N-ethylamino-carbonyl)-4-amino-n-butyl;
1-(S)-(1-N-ethylamino-carbonyl)-4-amino-n-butyl;
1-(R)-(1-N-ethylaminocarbonyl)-5-(t-butoxycarbonylamino)pent-5-yl;
1-(S)-(1-N-ethylaminocarbonyl)-5-(t-butoxycarbonylamino)pent-5-yl;
1-(R)-(1-N-ethylaminocarbonyl)-4-(NN-t-butoxycarbonylamino)-n-but-5-yl;
1-(S)-(1-N-ethylaminocarbonyl)-4-(NN-t-butoxycarbonylamino)-n-but-5-yl;
1-(R)-(1-N-ethylaminocarbonyl)-5-guanadino-n-pent-5-yl;
1-(S)-(1-N-ethylaminocarbonyl)-5-guanadino-n-pent-5-yl;
1-R,S-(1-N-ethylaminocarbonyl)-4-(NN-t-butoxycarbonyl)guanadino-n-but-1-yl;
1-(R)-(1-N-ethylaminocarbonyl)-5-(NN-t-butoxycarbonylamino)-n-pent-5-yl;
1-(S)-(1-N-ethylaminocarbonyl)-5-(NN-t-butoxycarbonylamino)-n-pent-5-yl;
2-(4-hydroxyphenyl)-1-(S)-(methoxycarbonyl)eth-1-yl;
2-(4-hydroxyphenyl)-1-(S)-(isopropoxycarbonyl)eth-1-yl;
2-[4-(imidazolin-2-yl)phenyl]-1-(R)-(pyrrolidin-1-ylcarbonyl)eth-1-yl;
1-(R)-(methoxycarbonyl)eth-1-yl;
1-(R)-(methoxycarbonyl)-2-(N-methylpiperidin-4-yl)eth-1-yl;
1-(R)-(methoxycarbonyl)-2-(N-methyl-1,2,3,6-tetrahydropyrid-4-yl)eth-1-yl;
2-(N-methylpiperidin-4-yl)-1-(R)-(pyrrolidin-N-ylcarbonyl)eth-1-yl;
2-(N-methyl-1,2,5,6-tetrahydropyrid-4-yl)-1-(R)-(pyrrolidin-N-ylcarbonyl) eth-1-yl;
2-(phenyl)-1-(S)-(pyrrolidin-N-ylcarbonyl)eth-1-yl;
2-(pyrid-4-yl)-1-(R)-(pyrrolidin-N-ylcarbonyl)eth-1-yl;
1-(R)-(pyrrolidin-N-ylcarbonyl)-2-(4-amidino)phenyl-eth-1-yl;
1-(S)-(pyrrolidin-N-ylcarbonyl)-2-(4-amidino)phenyl-eth-1-yl;
1-(R)-(pyrrolidin-N-ylcarbonyl)-5-amino-n-pent-1-yl;
1-(S)-(pyrrolidin-N-ylcarbonyl)-5-amino-n-pent-1-yl;
1-(R)-(pyrrolidin-N-ylcarbonyl)-2-(4-biphenyl)eth-1-yl;
1-(S)-(pyrrolidin-N-ylcarbonyl)-2-(4-biphenyl)eth-1-yl;
1-(R)-(pyrrolidin-N-ylcarbonyl-2-(4-iodophenyl)eth-1-yl;
1-(S)-(pyrrolidin-N-ylcarbonyl-2-(4-iodophenyl)eth-1-yl;
1-(R)-(pyrrolidin-N-carbonyl)-4-(t-butoxycarbonylamino)-n-but-1-yl;
1-(S)-(pyrrolidin-N-carbonyl)-4-(t-butoxycarbonylamino)-n-but-1-yl;
1-(S)-(pyrrolidin-N-ylcarbonyl)-2-[4-(2-imidazolin-2-yl)phenyl]eth-1-yl;
2-(R)-(pyrrolidin-N-ylcarbonyl-3-phenylprop-2-yl;
1-(R)-(pyrrolidin-N-ylcarbonyl)-2-[4-(N-methylpiperidin-2-yl) phenyl)]eth-1-yl;
1-(S)-(pyrrolidin-N-ylcarbonyl)-2-[4-(N-methylpiperidin-2-yl)phenyl)]eth-1-yl;
1-(R)-(pyrrolidin-N-ylcarbonyl)-2-[N-methyl-1,2,5,6-tetrahydropyridin-4-yl)-phen-4-yl)]eth-1-yl;
1-(S)-(pyrrolidin-N-ylcarbonyl)-2-[N-methyl-1,2,5,6-tetrahydropyridin-4-yl)-phen-4-yl)]eth-1-yl;
1-(R)-(pyrrolidin-N-ylcarbonyl)-2-[4-(piperidin-2-yl)cyclohexyl)]eth-1-yl;
1-(S)-(pyrrolidin-N-ylcarbonyl)-2-[4-(piperidin-2-yl)cyclohexyl)]eth-1-yl;
1-(R)-(pyrrolidin-N-ylcarbonyl)-2-[N-(phenyl)piperidin-4-yl)]eth-1-yl;
1-(S)-(pyrrolidin-N-ylcarbonyl)-2-[N-(phenyl)piperidin-4-yl)]eth-1-yl;
1-(R)-(pyrrolidin-N-ylcarbonyl)-2-[N-(pyridin-4-yl)piperidin-4-yl)]eth-1-yl;
1-(S)-(pyrrolidin-N-ylcarbonyl)-2-[N-(pyridin-4-yl)piperidin-4-yl)]eth-1-yl;
1-(R)-(pyrrolidin-N-ylcarbonyl)-2-[4-(pyridin-4-yl)phenyl)]eth-1-yl;
1-(S)-(pyrrolidin-N-ylcarbonyl)-2-[4-(pyridin-4-yl)phenyl)]eth-1-yl;
1-(R)-(pyrrolidin-N-ylcarbonyl)-2-[4-(pyrid-2-yl)phenyl]eth-1-yl;
1-(S)-(pyrrolidin-N-ylcarbonyl)-2-[4-(pyrid-2-yl)phenyl]eth-1-yl;
1-(R)-(pyrrolidin-N-ylcarbonyl)-2-[4-(pyrimidin-2-yl)phenyl]eth-1-yl;
1-(S)-(pyrrolidin-N-ylcarbonyl)-2-[4-(pyrimidin-2-yl)phenyl]eth-1-yl;
1-(R)pyrrolidin-N-ylcarbonyl)-2-[4-(N-t-butoxycarbonylpyrrol-2-yl)phenyl]eth-1-yl;
1-(S)-(pyrrolidin-N-ylcarbonyl)-2-[4-(N-t-butoxycarbonylpyrrol-2-yl)phenyl]eth-1-yl;
1-(S)-(t-butoxycarbonyl)-2-(4-hydroxyphenyl)eth-1-yl;
3-t-butoxycarbonyl-1-methoxycarbonylprop-1-yl; and
1-(R)-(t-butoxycarbonyl)-2-(phenyl)eth-1-yl.

Preferred X groups include hydrogen, bromine, chlorine and methyl.

In Formula (I) or Formula (II), the R⁵ and R⁶ groups (other than when both are hydrogen) results in two optical isomers. When R³ in Formula (I) or Formula (II) is other than hydrogen, two geometric isomers may exist. When R³ is hydrogen Formula (I) or Formula (II) are tautomers. In those cases where the compounds of Formula (I) or Formula (II) exist as tautomers, optical isomers or geometric isomers, the above formulas are intended to represent isomer mixtures as well as the individual isomeric bradykinin B₁ receptor antagonist or intermediate isomers, all of which are encompassed within the scope of this invention.

Further, references to the compounds of Formula (I) or Formula (II) with respect to pharmaceutical applications thereof are also intended to include pharmaceutically acceptable salts of the compounds of Formula (I) or Formula (II).

Compounds within the scope of this invention include those set forth in Table I as follows:

TABLE I

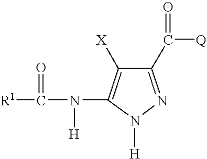

| Cpd # | R¹ | X | Q |
|---|---|---|---|
| 201 | 2-chlorophenyl | Br | (1-(R)-1-(N,N-dimethylaminocarbonyl) |
| | | | eth-1-yl)amino |
| 202 | 2-chlorophenyl | Br | (1-(S)-1-(N,N-dimethylaminocarbonyl) |
| | | | eth-1-yl)amino |
| 203 | 2-chlorophenyl | Br | (1-(S)-1-(phenyl)-1- |
| | | | (methoxycarbonyl)methylamino |
| 204 | 2-chlorophenyl | Br | (1-(S)-1-(phenyl)-1-carboxy)methylamino |
| 205 | 2-chlorophenyl | H | (1-(S)-1-(phenyl)-1- |
| | | | (methoxycarbonyl)methylamino |
| 206 | 2-chlorophenyl | Br | ((1-(S)-2-(phenyl)-1-(methoxycarbonyl) |
| | | | eth-1-yl)amino |
| 207 | 2-chlorophenyl | Br | (1-(R)-1-(phenyl)-1- |
| | | | (methoxycarbonyl)methyl)amino |
| 208 | 2-(3-methyl- | Br | ((1-(S)-1-(phenyl)-1-(methoxycarbonyl) |
| | phenylthio- | | methyl)amino |
| | methyl)phenyl | | |
| 209 | 2-chlorophenyl | Br | 1-(S)-1-(methoxycarbonyl)ent-1-yl)amino |
| 210 | 2-chlorophenyl | Br | (1-(S)-1-(phenyl)-1-(methylamino- |
| | | | carbonyl)methyl)amino |
| 211 | 2-chlorophenyl | Br | (1-(S)-1-(piperidin-1-ylcarbonyl) |
| | | | eth-1-yl)amino |
| 212 | 2-chlorophenyl | Br | (1-(R or S)-1-(N-morpholinocarbonyl) |
| | | | eth-1-yl)amino |
| 213 | 2-chlorophenyl | Br | (1-(R)-1-(piperidin-1-ylcarbonyl) |
| | | | eth-1-yl)amino |
| 214 | 2-chlorophenyl | Br | (1-(S)-1-(phenyl)-1-(piperidin-1- |
| | | | ylcarbonyL)methyl)amino |
| 215 | 2-chlorophenyl | Br | (1-(R)-1-(N-morpholinocarbonyl) |
| | | | eth-1-yl)amino |
| 216 | 2-chlorophenyl | Br | (1-(R)-1-(phenyl)-1-(piperidin-1- |
| | | | ylcarbonyl)methyl)amino |
| 217 | 2-chlorophenyl | H | (1-(R)-1-(piperidin-1-ylcarbonyl)eth-1- |
| | | | yl)amino |
| 218 | 2-chlorophenyl | Br | (1-(R)-1-(N,N-dimethylaminocarbonyl) |
| | | | prop-1-yl)amino |
| 219 | 2-chlorophenyl | Br | (1-(R)-1-(N-ethyl-N- |
| | | | methylaminocarbonyl)eth-1-yl)amino |
| 220 | 2-chlorophenyl | Br | (1-(R)-1-(N,N-diethylaminocarbonyl) |
| | | | eth-1-yl)amino |
| 221 | 2-chlorophenyl | Br | ((N-methyl-N-phenyl- |
| | | | aminocarbonyl)methyl)amino |
| 222 | 2-chlorophenyl | Br | ((1-methyl-piperizin-4- |
| | | | ylcarbonyl)methyl)amino |
| 223 | 2-chlorophenyl | Br | (1-(R)-1-(piperidin-1-ylcarbonyl)-3- |
| | | | (phenyl)prop-1-yl)amino |
| 224 | 2-chlorophenyl | Br | (1-(R or S)-1-(methoxycarbonyl)-2-(4- |
| | | | hydroxyphen-1-yl)eth-1-yl)amino |
| 225 | 2-chlorophenyl | Br | (1-(R or S)-1-(methoxycarbonyl)-1-(4- |
| | | | hydroxyphen-1-yl)methyl)amino |
| 226 | 2-chlorophenyl | Br | (1-(R)-1-(aminocarbonyl)-2-methylprop-1- |
| | | | yl)amino |
| 227 | 2-chlorophenyl | Br | (1-(S)-1-(aminocarbonyl)eth-1-yl)amino |
| 228 | 2-chlorophenyl | Br | (1-(S)-1-(phenyl)-1- |
| | | | (aminocarbonyl)methyl)amino |
| 229 | 2-chlorophenyl | Br | (1-(R)-1-(methoxycarbonyl)-2-(1H-indol- |
| | | | 3-yl)eth-1-yl)amino |
| 230 | 2-chlorophenyl | Br | (1-(R or S)-1-(methoxycarbonyl)-3- |
| | | | (methyl)but-1-yl)amino |
| 231 | 2-chlorophenyl | Br | (1-(R or S)-1-(methoxycarbonyl)eth-1- |
| | | | yl)amino |
| 232 | 2-chlorophenyl | Cl | (1-(R)-1-((piperidin-1-yl)carbonyl)eth-1- |
| | | | yl)amino |
| 233 | 2-chlorophenyl | Br | [(2-(dimethylaminocarbonyl)phen-1- |
| | | | ylaminocarbonyl)methyl]amino |

Particularly preferred compounds include the following compounds:
(R)-4-bromo-5-(2-chloro-benzoylamino)-1H-pyrazole-3-carboxylic acid (1-(N,N-dimethylaminocarbonyl)eth-1-yl)amide;
(S)-4-bromo-5-(2-chloro-benzoylamino)-1H-pyrazole-3-carboxylic acid (1-(N,N-dimethylaminocarbonyl)eth-1-yl)amide;
(S)-4-bromo-5-(2-chloro-benzoylamino)-1H-pyrazole-3-carboxylic acid [1-(phenyl)-1-(methoxycarbonyl)methyl]amide;
(S)-4-bromo-5-(2-chloro-benzoylamino)-1H-pyrazole-3-carboxylic acid [1-(phenyl)-1-(carboxy)methyl]amide;
(S)-5-(2-chloro-benzoylamino)-1H-pyrazole-3-carboxylic acid [1-(phenyl)-1-(methoxycarbonyl)methyl]amide;
(S)-4-bromo-5-(2-chloro-benzoylamino)-1H-pyrazole-3-carboxylic acid [2-(phenyl)-1-(methoxycarbonyl)eth-1-yl)]amide;
(R)-4-bromo-5-(2-chloro-benzoylamino)-1H-pyrazole-3-carboxylic acid [1-(phenyl)-1-(methoxycarbonyl)methyl]amide;
(S)-4-bromo-5-(2-m-tolylthiomethyl-benzoylamino)-1H-pyrazole-3-carboxylic acid [1-(phenyl)-1-(methoxycarbonyl)methyl]amide;
(S)-2-{[4-bromo-5-(2-chloro-benzoylamino)-1H-pyrazole-3-carboxylic acid [1-(methoxycarbonyl)pent-1-yl]amide;
(S)-4-bromo-5-(2-chloro-benzoylamino)-1H-pyrazole-3-carboxylic acid [1-(phenyl)-1-(methylaminocarbonyl)methyl]amide;
(S)-4-bromo-5-(2-chloro-benzoylamino)-1H-pyrazole-3-carboxylic acid [1-(piperidin-1-ylcarbonyl)eth-1-yl]amide;
4-bromo-5-(2-chloro-benzoylamino)-1H-pyrazole-3-carboxylic acid [1-(N-morpholinocarbonyl)eth-1-yl]amide;
(R)-4-bromo-5-(2-chloro-benzoylamino)-1H-pyrazole-3-carboxylic acid [1-(piperidin-1-ylcarbonyl)eth-1-yl]amide;
(S)-4-bromo-5-(2-chloro-benzoylamino)-1H-pyrazole-3-carboxylic acid [1-(phenyl)-1-(piperidin-1-ylcarbonyl)methyl]amide;
(R)-4-bromo-5-(2-chloro-benzoylamino)-1H-pyrazole-3-carboxylic acid [1-(N-morpholinocarbonyl)eth-1-yl]amide;
(R)-4-bromo-5-(2-chloro-benzoylamino)-1H-pyrazole-3-carboxylic acid [1-(phenyl)-1-piperidin-1-ylcarbonyl)methyl]aride;
(R)-5-(2-chloro-benzoylamino)-1H-pyrazole-3-carboxylic acid [1-(piperidin-1-ylcarbonyl)eth-1-yl]amide;
(R)-4-bromo-5-(2-chloro-benzoylamino)-1H-pyrazole-3-carboxylic acid [1-(N,N-dimethylaminocarbonyl)prop-1-yl]amide;
(R)-4-bromo-5-(2-chloro-benzoylamino)-1H-pyrazole-3-carboxylic acid [1-(N-ethyl-N-methylaminocarbonyl)eth-1-yl]amide;
(R)-4-bromo-5-(2-chloro-benzoylamino)-1H-pyrazole-3-carboxylic acid [1-(N,N-diethylaminocarbonyl)eth-1-yl]amide;
4-bromo-5-(2-chloro-benzoylamino)-1H-pyrazole-3-carboxylic acid [N-methyl-N-phenyl-aminocarbonyl)methyl]amide;
4-bromo-5-(2-chloro-benzoylamino)-1H-pyrazole-3-carboxylic acid [(1-methyl-piperizin-4-ylcarbonyl)methyl]amide;
(R)-4-bromo-5-(2-chloro-benzoylamino)-1H-pyrazole-3-carboxylic acid [1-(piperidin-1-ylcarbonyl)-3-(phenyl)prop-1-yl]amide;
4-bromo-5-(2-chloro-benzoylamino)-1H-pyrazole-3-carboxylic acid [1-(methoxycarbonyl)-2-(4-hydroxyphen-1-yl)eth-1-yl]amide;
4-bromo-5-(2-chloro-benzoylamino)-1H-pyrazole-3-carboxylic acid [1-(methoxycarbonyl)-1-(4-hydroxyphen-1-yl)methyl]amide;
(R)-4-bromo-5-(2-chloro-benzoylamino)-1H-pyrazole-3-carboxylic acid [1-(aminocarbonyl)-2-(methyl)prop-1-yl]amide;
(S)-4-bromo-5-(2-chloro-benzoylamino)-1H-pyrazole-3-carboxylic acid [1-(aminocarbonyl)eth-1-yl]amide;
(S)-4-bromo-5-(2-chloro-benzoylamino)-1H-pyrazole-3-carboxylic acid [1-(phenyl)-1-(aminocarbonyl)methyl]amide;
(R)-4-bromo-5-(2-chloro-benzoylamino)-1H-pyrazole-3-carboxylic acid [1-(methoxycarbonyl)-2-(1H-indol-3-yl)eth-1-yl]amide;
4-bromo-3-(2-chloro-benzoylamino)-1H-pyrazole-5-carboxylic acid [1-(methoxycarbonyl)-3-methylbut-1-yl]amide;
4-bromo-3-(2-chloro-benzoylamino)-1H-pyrazole-5-carboxylic acid [1-(methoxycarbonyl)eth-1-yl]amide;
(R)-4-chloro-5-(2-chloro-benzoylamino)-1H-pyrazole-3-carboxylic acid [1-(piperidin-1-ylcarbonyl)eth-1-yl]amide;
4-bromo-5-(2chlorobenzoylamino)-1H-pyrazole-3-carboxylic acid [(2-(dimethylaminocarbonyl)phen-1-ylaminocarbonyl)methyl]amide; or
pharmaceutically acceptable salts thereof.

Compounds of Formula (I) and Formula (II) can be employed as selective antagonists of the bradykinin B₁ over the bradykinin B₂ receptor.

This invention further provides a method for selectively inhibiting bradykinin B₁ receptor over bradykinin B₂ receptor in a biological sample comprising both the bradykinin B₁ and B₂ receptors which method comprises contacting an inhibiting effective amount of a compound of claim 1 or mixture thereof to the biological sample.

The present invention further provides a pharmaceutical composition comprising a pharmaceutically acceptable carrier and an amount of a compound of Formula (I) or Formula (II) or mixtures thereof effective to treat or palliate adverse symptoms in mammals mediated by bradykinin B₁ receptor.

The present invention further provides a method for treating or palliating adverse symptoms in a mammal mediated at least in part by bradykinin B₁ receptor which method comprises administering a therapeutically effective amount of a compound of Formula (I) or Formula (II) or mixtures thereof or as is more generally the case the pharmaceutical composition.

The present invention further provides a pharmaceutical composition comprising a pharmaceutically acceptable carrier and a therapeutically effective amount of a compound of Formula (I) or Formula (II) or mixtures thereof to treat or palliate adverse symptoms in a mammal associated with up-regulating bradykinin B₁ receptor following tissue damage or inflammation.

The present invention further provides a method for treating or palliating adverse symptoms in a mammal associated with tissue damage or inflammation which method comprises administering a therapeutically effective amount of a compound of Formula (I) or Formula (II) or mixtures thereof or as is more generally the case the pharmaceutical composition.

The present invention further provides a method for treating or palliating adverse symptoms associated with the presence or secretion of bradykinin B₁ receptor agonists in a mammal which method comprises administering a therapeutically effective amount of a compound of Formula (I) or Formula (II) or mixtures thereof or as is more generally the case the pharmaceutical composition.

The present invention provides a method for treating or ameliorating pain, inflammation, septic shock or the scarring process in a mammal mediated at least in part by bradykinin B₁ receptor which method comprises administering a therapeutically effective amount of a compound of Formula (I) or Formula (II) or mixtures thereof or as is more generally the case the pharmaceutical composition.

The present invention provides a method for treating or ameliorating adverse symptoms in a mammal associated with burns, perioperative pain, migraine, shock, central nervous system injury, asthma, rhinitis, premature labor, inflammatory arthritis, inflammatory bowel disease or neuropathic pain which method comprises administering a therapeutically effective amount of a compound of Formula (I) or Formula (II) or mixtures thereof or as is more generally the case the pharmaceutical composition.

The present invention further provides a method for treating or palliating adverse symptoms associated with the presence or secretion of bradykinin B₁ receptor agonists in a mammal which method comprises administering a therapeutically effective amount of a compound of Formula (I) or Formula (II) or mixtures thereof or as is more generally the case the pharmaceutical composition.

The invention also provides a method for determining bradykinin B₁ receptor agonist levels in a biological sample which method comprises contacting said biological sample with a compound of Formula (I) or Formula (II), at a predetermined concentration.

### DETAILED DESCRIPTION OF THE PREFERRED EMBODIMENTS

As noted above, this invention is directed to certain 3-amido-5-substituted pyrazole derivatives and related compounds which are useful as bradykinin B₁ receptor antagonists to relieve adverse symptoms in mammals mediated, at least in part, by bradykinin B₁ receptor including pain, inflammation, septic shock, the scarring process, etc. However, prior to describing this invention in further detail, the following terms will first be defined.

Definitions

Unless otherwise expressly defined with respect to a specific occurrence of the term, the following terms as used herein shall have the following meanings regardless of whether capitalized or not:

The term “alkyl” or “alk” refers to monovalent alkyl groups having from 1 to 15 carbon atoms and more preferably 1 to 6 carbon atoms and includes both straight chain and branched chain alkyl groups. This term is exemplified by groups such as methyl, t-butyl, n-heptyl, octyl and the like. The term C₁₋₄alkyl refers to all groups with from 1 to 4 carbon atoms.

“Substituted alkyl” refers to an alkyl group, of from 1 to 15 carbon atoms, more preferably, 1 to 6 carbon atoms, having from 1 to 5 substituents, preferably 1 to 3 substituents, independently selected from the group consisting of alkoxy, substituted alkoxy, acyl, acylamino, thiocarbonylamino, acyloxy, amino, substituted amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxylaryl, substituted aryloxyaryl, cyano, halogen, hydroxyl, nitro, oxo (>C═O), thioxo (>C═S), carboxyl, carboxyl esters, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂—OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, and —NRS(O)₂—NR-substituted heterocyclic where R is hydrogen or alkyl.

“Alkoxy” refers to the group “alkyl-O—” which includes, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Substituted alkoxy” refers to the group “substituted alkyl-O—”.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)— provided that a nitrogen atom of the heterocyclic or substituted heterocyclic is not bound to the —C(O)— group.

“Amino” refers to the group —NH₂.

“Substituted amino” refers to the group —NR⁴¹R⁴¹, where each R⁴¹ group is independently selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic, provided that both R⁴¹ groups are not hydrogen; or the R⁴¹ groups can be joined together with the nitrogen atom to form a heterocyclic or substituted heterocyclic ring.

The “acylamino” or as a prefix “carbamoyl” or “carboxamide” or “substituted carbamoyl” or “substituted carboxamide” refers to the group —C(O)NR⁴²R⁴² where each R⁴² is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R⁴² is joined to form together with the nitrogen atom a heterocyclic or substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiocarbonylamino” or as a prefix “thiocarbamoyl”, “thiocarboxamide” or “substituted thiocarbamoyl” or “substituted thiocarboxamide” refers to the group —C(S)NR⁴³R⁴³ where each R⁴³ is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R⁴³ is joined to form, together with the nitrogen atom a heterocyclic or substituted heterocyclic wherein alkyl, substituted alkyl, alkenyl, substituted alkenyl, alknyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” refers to the groups acyl-O— where acyl is as defined herein.

“Alkenyl” refers to alkenyl group having from 2 to 10 carbon atoms and more preferably 2 to 6 carbon atoms and having at least 1 and preferably from 1-2 sites of alkenyl unsaturation.

“Substituted alkenyl” refers to alkenyl groups as defined herein, having from 1 to 5 substituents, preferably 1 to 3 substituents, independently selected from the group of substituents defined for substituted alkyl provided that the hydroxyl, thio, oxo or thioxo groups are not attached to a vinyl carbon atom.

“Alkynyl” refers to alkynyl group having from 2 to 10 carbon atoms and more preferably 3 to 6 carbon atoms and having at least 1 and preferably from 1-2 sites of alkynyl unsaturation.

“Substituted alkynyl” refers to alkynyl groups, as defined herein, having from 1 to 5, preferably 1 to 3 substituents, selected from the same group of substituents as defined for substituted alkyl provided that the hydroxyl, thio, oxo or thioxo groups are not attached to an acetylenic carbon atom.

“Amidino” refers to the group H₂NC(═NH)— and the term “alkylamidino” refers to compounds having 1 to 3 alkyl groups (e.g., alkylHNC(═NH)—).

“Thioamidino” refers to the group R⁴⁴SC(═NH)— where R⁴⁴ is hydrogen or alkyl where alkyl is as defined herein.

“Aminoacyl” refers to the groups —NR⁴⁵C(O)alkyl, —NR⁴⁵C(O)substituted alkyl, —NR⁴⁵C(O)cycloalkyl, —NR⁴⁵C(O)substituted cycloaLkyl, —NR⁴⁵C(O)alkenyl, —NR⁴⁵C(O)substituted alkenyl, —NR⁴⁵C(O)alkynyl, —NR⁴⁵C(O)substituted alkynyl, —NR⁴⁵C(O)aryl, —NR⁴⁵C(O)substituted aryl, —NR⁴⁵C(O)heteroaryl, —NR⁴⁵C(O)substituted heteroaryl, —NR⁴⁵C(O)heterocyclic, and —NR⁴⁵C(O)substituted heterocyclic where R⁴⁵ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are defined herein.

“Aminocarbonyloxy” refers to the groups —NR⁴⁶C(O)O-alkyl, —NR⁴⁶C(O)O-substituted alkyl, —NR⁴⁶C(O)O-alkenyl, —NR⁴⁶C(O)O-substituted alkenyl, —NR⁴⁶C(O)O-alkynyl, —NR⁴⁶C(O)O-substituted alkynyl, —NR⁴⁶C(O)O-cycloalkyl, —NR⁴⁶C(O)O-substituted cycloalkyl, —N⁴⁶C(O)O-aryl, —NR⁴⁶C(O)O-substituted aryl, —NR⁴⁶C(O)O-heteroaryl, —NR⁴⁶C(O)O-substituted heteroaryl, —NR⁴⁶C(O)O-heterocyclic, and —NR⁴⁶C(O)O-substituted heterocyclic where R⁴⁶ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxycarbonylamino” or as a prefix “carbamoyloxy” or “substituted carbamoyloxy” refers to the groups —OC(O)NR⁴⁷R⁴⁷ where each R⁴⁷ is independently hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic or where each R⁴⁷ is joined to form, together with the nitrogen atom a heterocyclic or substituted heterocyclic and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxythiocarbonylamino” refers to the groups —OC(S)NR⁴⁸R⁴⁸ where each R⁴⁸ is independently hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic or where each R⁴⁸ is joined to form, together with the nitrogen atom a heterocyclic or substituted heterocyclic and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” refers to the group —NR⁴⁹C(O)NR⁴⁹— where R⁴⁹ is selected from the group consisting of hydrogen and alkyl.

“Aminothiocarbonylamino” refers to the group —NR⁵⁰C(S)NR⁵⁰— where R⁵⁰ is selected from the group consisting of hydrogen and alkyl.

“Aryl” or “Ar” refers to an aromatic carbocyclic group of from 6 to 14 carbon atoms having a single ring (i.e., monocyclic) (e.g., phenyl) or multiple condensed rings (e.g., naphthyl or anthryl) which condensed rings may or may not be aromatic (e.g., 2-benzoxazolinone, 2H-1,4-benzoxazin-3(4H)-one, and the like). When at least one of the rings in the fused multicyclic ring system is non-aromatic such as cycloalkyl, cycloalkenyl, heterocyclic or heteroaryl, the point of attachment of the aryl group to the core structure is on one of the aryl atoms. Preferred aryls include phenyl and naphthyl.

“Substituted aryl” refers to aryl groups, as defined herein, which are substituted with from 1 to 4, preferably 1-3, substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, substituted amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxyl esters, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OS(O)₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O):-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl.

“Aryloxy” refers to the group aryl-O— which includes, by way of example, phenoxy, naphthoxy, and the like.

“Substituted aryloxy” refers to substituted aryl-O— groups.

“Aryloxyaryl” refers to the group -aryl-O-aryl.

“Substituted aryloxyaryl” refers to aryloxyaryl groups substituted with from 1 to 4, preferably 1-3 substituents on either or both aryl rings independently selected from the same group consisting of substituents as defined for substituted aryl.

“Carboxyl” refers to the group —COOH and pharmaceutically acceptable salts thereof.

“Carboxyl esters” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-cycloalkyl, —C(O)O-substituted cycloalkyl, —C(O)O-aryl, —C(O)O-substituted aryl, —C(O)O-aryl, —C(O)O-substituted aryl, —C(O)O-heteroaryl, —C(O)O-substituted heteroaryl, —C(O)Oheterocyclic, —C(O)O-substituted heterocyclic, and the like.

“Cycloalkyl” refers to cyclic alkyl groups of from 3 to 20 carbon atoms having a single or multiple cyclic rings including, by way of example, cyclopropyl, cyclobutyl, cyclopentyl, cyclooctyl, adamantanyl, and the like. Cycloalkyl groups of the present invention also include fused multicyclic rings wherein one or more of the rings within the multicyclic ring system are aryl, cycloalkenyl, heteroaryl, and/or heterocyclic, as long as the point of attachment to the core or backbone of the structure is on the non-aromatic cycloalkyl ring.

“Cycloalkenyl” refers to cyclic alkenyl groups of from 4 to 20 carbon atoms having single or multiple unsaturation and having a single or multiple cyclic unsaturated but not aromatic rings. Suitable cycloalkenyl groups include, by way of example, cyclopentenyl, cyclooctenyl, and the like. Cycloalkenyl groups of the present invention also include fused multicyclic rings wherein one or more of the rings within the multicyclic ring system are aryl, heteroaryl, cycloalkyl and/or heterocyclic, as long as the point of attachment to the core or backbone of the structure is on the non-aromatic cycloalkenyl ring.

“Substituted cycloalkyl” and “substituted cycloalkenyl” refer to cycloalkyl and cycloalkenyl groups, as defined herein, having from 1 to 5, preferably 1-3 substituents independently selected from the same group of substituents as defined for substituted alkyl.

“Cycloalkoxy” refers to —O-cycloalkyl groups where cycloalkyl is as defined herein.

“Substituted cycloalkoxy” refers to —O-substituted cycloalkyl groups where substituted cycloalkyl is as defined herein.

“Guanidino” or “substituted guanidino” refers to the groups —NR⁵²C(═NR⁵²)NR⁵²R⁵² where each R⁵² is independently hydrogen or alkyl.

“Guanidinosulfone” refers to the groups —NR⁵³C(═NR⁵³)NRSO₂-alkyl, —NR⁵³C(═NR⁵³)NR⁵³SO₂-substituted alkyl, —NR⁵³C(═NR⁵³)NR⁵³SO₂-alkenyl, —NR⁵³C(═NR⁵³)NR⁵³SO₂-substituted alkenyl, —NR⁵³C(═NR⁵³)NR⁵³SO₂-alkynyl, —NR⁵³C(═NR⁵³)NR⁵³SO₂-substituted alkynyl, —NR⁵³C(═NR⁵³)NR⁵³SO₂-aryl, —NR⁵³C(═NR⁵³)NR⁵³SO₂-substituted aryl, —NR⁵³C(═NR⁵³)NR⁵³SO₂-cycloalkyl, —NR⁵³C(═NR⁵³)NR⁵³SO₂-substituted cycloalkyl, —NR⁵³C(═NR⁵³)NR⁵³SO₂-heteroaryl, —NR⁵³C(═NR⁵³)NR⁵³SO₂-substituted heteroaryl, —NR⁵³C(═NR⁵³)NR⁵³SO₂-heterocyclic, and —NR⁵³C(═NR⁵³)NR⁵³SO₂-substituted heterocyclic where each R⁵³ is independently hydrogen and alkyl.

“Halo” or “halogen” refers to fluoro, chloro, bromo and iodo.

“Heteroaryl” refers to an aromatic group of from 2 to 10 ring carbon atoms and 1 to 4 ring heteroatoms selected from oxygen, nitrogen and sulfur within the ring. Such heteroaryl groups can have a single ring (i.e., monocyclic) (e.g., pyridyl or furyl) or multiple condensed rings (e.g., indolizinyl or benzothienyl) which may be non-heteroaryl. When at least one of the rings in the fused multicyclic ring system is non-heteroaryl such as aryl, cycloalkyl, cycloalkenyl or heterocyclic, the point of attachment of the heteroaryl group to the core structure is on one of the heteroaryl atoms. Preferred heteroaryls include pyridyl, pyrrolyl, indolyl and furyl.

“Substituted heteroaryl” refers to heteroaryl groups, as defined above, which are substituted with from 1 to 3 substituents independently selected from the same group of substituents as defined for “substituted aryl”.

“Heteroaryloxy” refers to the group —O-heteroaryl and “substituted heteroaryloxy” refers to the group —O-substituted heteroaryl where heteroaryl and substituted heteroaryl are as defined above.

“Heterocycle” or “heterocyclic” refers to a saturated or unsaturated, but not heteroaromatic, group having a single ring or multiple condensed rings, from 2 to 20 ring carbon atoms and from 1 to 4 ring hetero atoms selected from nitrogen, sulfur or oxygen within the ring. “Heterocycle” or “heterocyclic” groups of the present invention also include fused multicyclic rings wherein one or more of the rings within the multicyclic ring system is not heterocyclic (e.g., cycloalkyl, cycloalkenyl, aryl or heteroaryl), as long as the point of attachement to the core or backbone of the structure is on the heterocyclic ring.

“Substituted heterocyclic” refers to heterocycle groups, as defined above, which are substituted with from 1 to 3 substituents independently selected from the group consisting of oxo (═O), thioxo (═S), plus the same group of substituents as defined for substituted aryl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole; carboline, phenanthridine, acridine, phenanthroline, isothiazole, phenazine, isoxazole, phenoxazine, phenothiazine, imidazolidine, imidazolinie, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholino, thiomorpholino, piperidinyl, pyrrolidine, tetrahydrofuranyl, and the like.

“Heterocyclyloxy” refers to the group —O-heterocyclic and “substituted heterocyclyloxy” refers to the group —O-substituted heterocyclic where heterocyclic and substituted heterocyclyoxy are as defined above.

“Thiol” refers to the group —SH.

“Thioalkyl” or “thioalkoxy” refer to the groups —S-alkyl.

“Substituted thioalkyl” and “substituted thioalkoxy” refer to the group —S-substituted alkyl.

“Thiocycloalkyl” refers to the groups —S-cycloalkyl.

“Substituted thiocycloalkyl” refers to the group —S-substituted cycloalkyl.

“Thioaryl” refers to the group —S-aryl and “substituted thioaryl” refers to the group —S-substituted aryl.

“Thioheteroaryl” refers to the group —S-heteroaryl and “substituted thioheteroaryl” refers to the group —S-substituted heteroaryl.

“Thioheterocyclic” refers to the group —S-heterocyclic and “substituted thioheterocyclic” refers to the group —S-substituted heterocyclic.

Amino acid refers to any of the naturally occurring amino acids, as well as synthetic analogs (e.g., D-stereoisomers of the naturally occurring amino acids, such as D-threonine) and derivatives thereof. α-Amino acids comprise a carbon atom to which is bonded an amino group, a carboxyl group, a hydrogen atom, and a distinctive group referred to as a “side chain”. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine and tryptophan), substituted arylalkyl (e.g., as in tyrosine), and heteroarylalkyl (e.g., as in histidine). Unnatural amino acids are also known in the art, as set forth in, for example, Williams (ed.), Synthesis of Optically Active alpha.-Amino Acids, Pergamon Press (1989); Evans et al., J. Amer. Chem. Soc., 112:4011-4030 (1990); Pu et al., J. Org. Chem., 56:1280-1283 (1991); Williams et al., J. Amer. Chem. Soc., 113:9276-9286 (1991); and all references cited therein.

Naturally and non-naturally occurring amino acid side chains, R⁵ and/or R⁶, are preferably selected from the group consisting of alkyl, substituted alkyl, alkenyl, substituted alkenyl, cycloalkyl, substituted cycloalkyl, cycloalkenyl, substituted cycloalkenyl, aryl, substituted aryl, heteroaryl, substituted heteroaryl, heterocyclic and substituted heterocyclic.

“Pharmaceutically acceptable salt” refers to pharmaceutically acceptable salts of a compound of Formula (I) or Formula (II) which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “pharmaceutically acceptable prodrugs” refers to art recognized modifications to one or more functional groups which functional groups are metabolized in vivo to provide a compound of this invention or an active metabolite thereof. Such functional groups are well known in the art including acyl groups for hydroxyl and/or amino substitution, esters of mono-, di- and tri-phosphates wherein one or more of the pendent hydroxyl groups have been converted to an alkoxy, a substituted alkoxy, an aryloxy or a substituted aryloxy group, and the like.

It is understood that the substitution patterns defined herein do not include any chemically impossible substitution patterns. Moreover, when a group is defined by the term “substituted” such as substituted aryl and a possible substituent is the substituted group itself, e.g., substituted aryl substituted with substituted aryl, it is not intended that such substitution patterns be repeated indefinitely such as to produce a polymer,e.g., (substituted aryl)ₙ. Accordingly, in all cases, the maximum number of repetitions is 4. That is too say that n is an integer from 1 to 4.

Compound Preparation

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such, conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

The compounds of this invention will typically contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), March's Advanced Organic Chemistry, (John Wiley and Sons, 5^{th} Edition) and Larock's Comprehensive Organic Transformations 2^{nd} Edition (VCH Publishers Inc., 1999).

Specifically, the substituted pyrazoles and various intermediates useful in the preparation of substituted pyrazoles are preferably prepared as shown in the following Schemes.

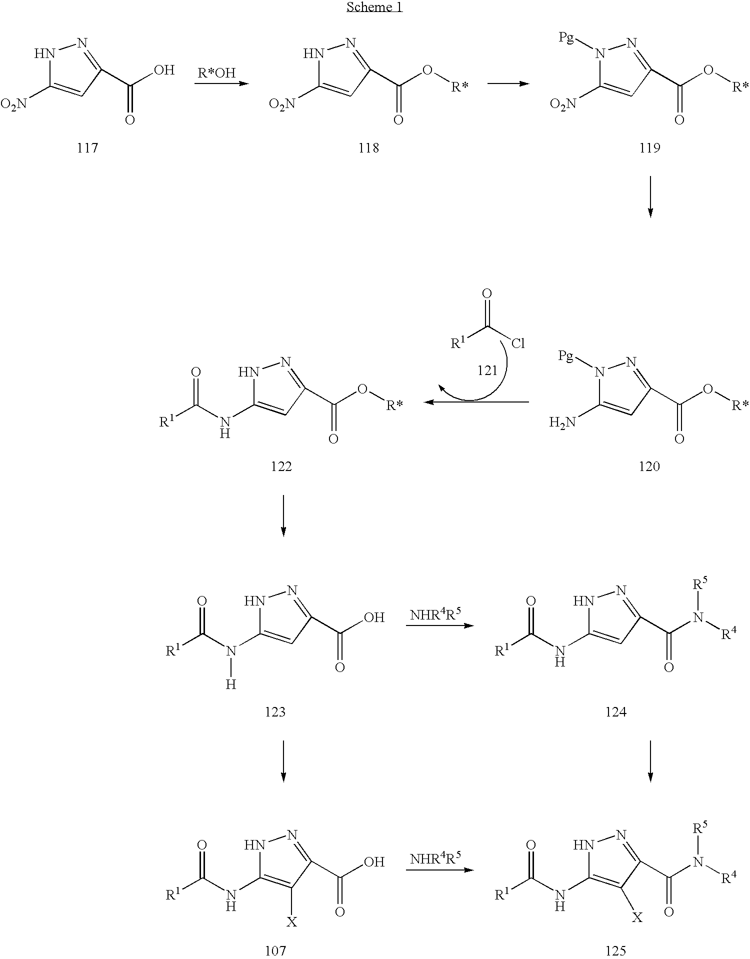

Specifically, in Scheme 1 (where R¹, R³, R⁴, R⁵, X, are as defined above and R* is alkyl), commercially available 3-nitro-5-carboxyl pyrazole, compound 117, is esterified under conventional conditions using a suitable alcohol, such as methanol, in acidic medium to provide for the corresponding ester, 118. The particular alcohol employed is not critical and is typically selected based on ease of synthesis and costs. The reaction is preferably conducted at an elevated temperature of from about 25 to about 100° C. until the reaction is substantially complete, which is typically 2 to 12 hours. The resulting product, compound 118, can be recovered by conventional methods, such as chromatography, filtration, crystallization, and the like or, alternatively, used in the next step without purification and/or isolation.

The 3-nitro-5-carboxyl ester pyrazole, compound 118, is protected with a conventional protecting group, Pg, under conventional conditions to afford compound 119. The selected protecting group is one that is removed under conditions other than hydrogenation. A preferred protecting group is the Boc group.

The nitro group of the protected 3-nitro-5-carboxyl ester pyrazole, compound 119, is reduced to an amine using standard reduction reactions. For example, compound 119 is reacted with hydrogen gas at about 10 to 60 psi, in the presence of a suitable catalyst such as Pd on carbon to afford the corresponding amine, compound 120. The reaction is preferably conducted at a temperature of from about 20 to about 80° C. until the reaction is substantially complete, which is typically 1 to 5 hours. The resulting amine, compound 120, can be recovered by conventional methods, such as chromatography, filtration, crystallization, and the like or, alternatively, used in the next step without purification and/or isolation.

The 3-amino-5-carboxyl ester pyrazole, compound 120, is acylated under conventional conditions by reaction with at least a stoichiometric amount and preferably an excess of a desired acyl chloride, compound 121. The reaction is preferably conducted in the presence of a conventional activating agent such as DMAP in the presence of a base such as pyridine that scavenges the acid generated. The reaction is preferably conducted in an inert solvent such as dichloromethane, chloroform and the like although a liquid base such as pyridine can be employed as the solvent and to scavenge the acid generated. The reaction is preferably conducted at a temperature of from about −5 to about 35° C. until the reaction is substantially complete, which is typically 2 to 12 hours. The resulting product, compound 122, is obtained after a standard deprotection reaction, and can be recovered by conventional methods, such as chromatography, filtration, crystallization, and the like or, alternatively, used in the next step without purification and/or isolation.

Hydrolysis of compound 122, using conventional conditions such as lithium hydroxide and water in methanol and/or THF, affords the 3-(R¹CO)-5-carboxylic acid pyrazole, compound 123.

Compound 123 is functionalized at the 4-position of the pyrazole ring by conventional methods to provide for compound 107. For example, when X is halo, compound 123 is contacted with at least a stoichiometric amount of a suitable halogenation agent such as N-halo succinimide, bromine, and the like. The reaction is conducted in an inert diluent such as dimethylformamide, dichloromethane, and the like at a temperature sufficient to effect halogenation. Typically, the reaction is conducted at from about 0° to about 40° C. until the reaction is substantially complete which typically occurs in about 0.1 to 10 hours. The resulting product, compound 107, can be recovered by conventional methods, such as chromatography, filtration, crystallization, and the like or, alternatively, used in the next step without purification and/or isolation.

Subsequently, the carboxylic acid group of compound 107 is amidated using at least a stoichiometric amount and preferably an excess of a suitable amine, HNR⁴R⁵, under conventional conditions well known in the art preferably using an activating agent to effect coupling such as HOBT, EDC.HCL, NMM and the like. The resulting compound 125 can be recovered by conventional methods, such as chromatography, filtration, crystallization, and the like.

Alternatively, compound 123 may be amidated as described above to form compound 124, which can be recovered by conventional methods, such as chromatography, filtration, crystallization, and the like. Compound 124 can be functionalized at the 4-position of the pyrazole ring by conventional methods to provide for compound 125 using the same methods described for the conversion of compound 123 to compound 107.

In an alternative synthetic embodiment, compounds of Formula (I) or Formula (II) where X is alkyl can be prepared as shown in Scheme 2 below:

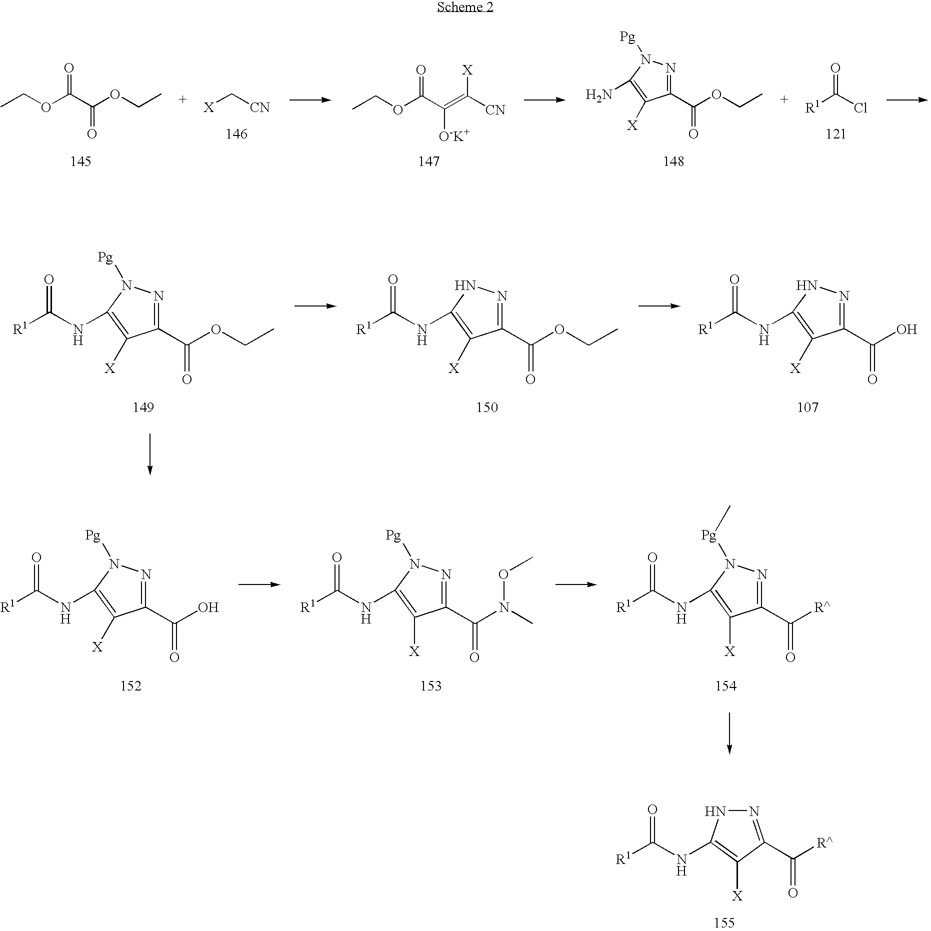

Specifically, in Scheme 2, wherein R¹ is defined above, commercially available oxalic acid diethyl ester, compound 145, is combined with at least a stoichiometric amount of an alkylnitrile, compound 146, in the presence of a suitable base such as potassium ethoxide in ethanol. The reaction is preferably maintained at a temperature of from about 60° C. to about 100° C. until the reaction is substantially complete, which is typically 12 to 24 hours. The resulting product, compound 147, can be recovered by conventional methods, such as chromatography, filtration, crystallization, and the like or, alternatively, used in the next step without purification and/or isolation.

Compound 147 is then cyclized using a slight excess of t-butyl hydrazine hydrochloride (not shown) in ethanol. The reaction is preferably conducted at elevated temperatures and pressures such as a temperature of from about 75 to about 150° C. and a pressure of from about 1 to 10 atm until the reaction is substantially complete, which is typically 12 to 24 hours. The resulting product, compound 148, can be recovered by conventional methods, such as chromatography, filtration, crystallization, and the like or, alternatively, used in the next step without purification and/or isolation.

Reaction of compound 148 proceeds in the manner described above for compound 120 to provide for compounds of Formula (I) or Formula (II) where X is alkyl.

Scheme 2 further illustrates derivatization of the carboxyl group of the 2-(Pg)-3-(—NHC(O)R¹)-4-(X)-5-carboxyl pyrazole, compound 149. Specifically, conventional hydrolysis of the ester provides for compound 152 which is then converted to the methoxymethylamide by reaction with commercially available N—O-dimethyl-hydroxylamine hydrochloride under conventional coupling conditions in a suitable inert diluent such as tetrahydrofuran, dioxane, and the like optionally in the presence of an activating agent. The reaction is maintained under conditions sufficient to afford compound 153 including, for example, a temperature of from about 0 to about 40° C. for a period of from 12 to 24 hours. The resulting product, compound 153, can be recovered by conventional methods, such as chromatography, filtration, crystallization, and the like or, alternatively, used in the next step without purification and/or isolation.

Compound 153 is then derivatized by contact with at least a stoichiometric amount, and preferably an excess, of Rˆ—Li where Rˆ is selected from the group consisting of alkyl, substituted alkyl, cycloalkyl, substituted cycloalkyl, heterocycloalkyl, substituted heterocycloalkyl, aryl, substituted aryl, heteroaryl and substituted heteroaryl. The reaction is typically conducted in an inert solvent such as tetrahydrofuran, dioxane, and the like at a reduced temperature of from about 0° C. to about −78° C. for a period of time sufficient for substantial reaction completion which typically occurs in about 12 to 24 hours. The product, compound 155, can be recovered by conventional methods, such as chromatography, filtration, crystallization, and the like

The starting materials employed in the reactions described above are either commercially available and/or can be prepared by methods well known in the art. For example, acid halides of the formula R¹C(O)X are readily prepared from the corresponding carboxylic acid by reaction with, e.g., oxalyl halide, thionyl halide and the like. Acids of the formula R¹C(O)OH are extremely well known and include aromatic acids (e.g., R¹ is aryl)

Alternatively, o-(Ar—S—CH₂-)benzoyl chloride, compound 143, can be prepared as illustrated in Scheme 3 below where Ar is an aryl, substituted aryl, heteroaryl, substituted heteroaryl, heterocyclic, and substituted heterocyclic:

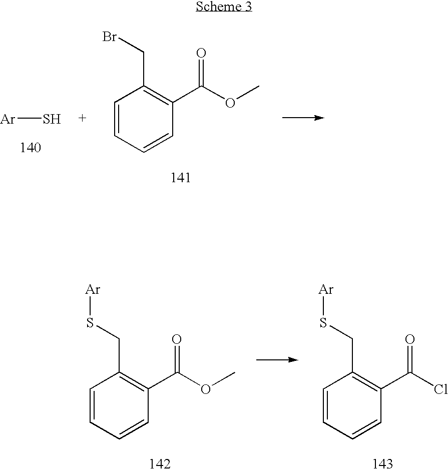

Specifically, compound 140 is coupled to o-bromomethyl-benzoic acid methyl ester, compound 141 (prepared as per Dvornikovs J. Org. Chem, 2002, 67, 2160), in the presence of about 30 equivalents potassium carbonate in DMF. This reaction is typically conducted at a temperature of from about 0 to about 30° C. until the reaction is substantially complete, which is typically 1 to 15 days. The resulting product, compound 142, can be recovered by conventional methods, such as chromatography, filtration, crystallization, and the like or, alternatively, used in the next step without purification and/or isolation.

o-(Ar—S—CH₂-)benzoyl chloride, compound 143, is then prepared by conventional hydrolysis of the methyl ester in compound 142 followed by conventional conversion of the carboxyl group to the carboxyl halide using, e.g., oxalyl halide in the presence of a base to scavenge the acid generated. The reaction is typically conducted in an inert solvent such as dichloromethane. This reaction is typically run at a temperature of about −20 to about 10° C. until the reaction is substantially complete, which is typically 1 to 12 hours. The resulting product, compound 143, can be recovered by conventional methods, such as filtration, crystallization, and the like or, alternatively, used in the next step without purification and/or isolation.

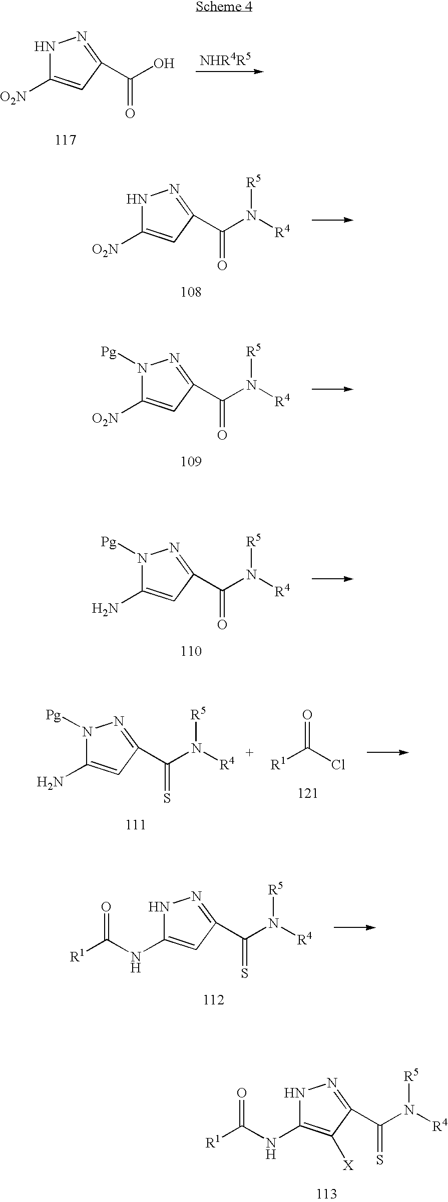

In one embodiment, Z of the substituted pyrazoles of Formula I is sulfur. These substituted pyrazoles are prepared as shown in Scheme 4, where Pg, X, R⁴, R⁵ and R¹ are as defined herein above.

Specifically, in Scheme 4, commercially available 3-nitro-5-carboxyl pyrazole, compound 117, is coupled to an amine using conventional conditions, for example, by using an activating agent such as HOBT, EDC.HCl, NMM and the like to effect coupling as described herein above. The resulting compound 108 can be recovered by conventional methods such as chromatography, filtration, crystallization and the like.

The 3-nitro-5-carboxyl amide pyrazole, compound 108, is protected with a protecting group, Pg, under conventional conditions to afford compound 109. The selected protecting group is one that is removed under conditions other than hydrogenation. A preferred protecting group is the Boc group.

The nitro group of the protected 3-nitro-5-carboxyl amide pyrazole, compound 109, is reduced to an amine using standard reduction reactions. For example, compound 109 is reacted with hydrogen gas at about 10 to 60 psi, in the presence of a suitable catalyst such as Pd on carbon to afford the corresponding amine, compound 110.

The 3-amino-5-carboxyl amide pyrazole, compound 110, is converted to the thioamide, compound 111, under conventional conditions known in the art. Formation of thioamides from amides can be accomplished using a number of known methods including the use of P₄S₁₀ or Lawesson's reagent as well as other methods know in the art such as those illustrated by Ernst Schaumann in Comprehensive Organic Synthesis Barry M. Trost, Ed.; Pergamon Press: Oxford, 1991; Vol. 6, Chapter 2.4, which is incorporated herein by reference in its entirety.

The 3-amino-5-thiocarboxyl amide pyrazole, compound 111, is acylated under conventional conditions by reaction with a desired acyl chloride, compound 121. The reaction is preferably conducted in the presence of a conventional activating agent such as DMAP in the presence of a base such as pyridine that scavenges the acid generated. The reaction is preferably conducted in an inert solvent such as dichloromethane, chloroform and the like. Alternatively a liquid base such as pyridine can be employed as the solvent and to scavenge the acid generated. The reaction is typically conducted at a temperature of about −5 to about 35° C. until completion, usually about 2 to about 12 hours. The resulting product, compound 112, is obtained after a standard deprotection reaction, and can be recovered by conventional methods, such as chromatography, filtration, crystallization, and the like or, alternatively, used in the next step without purification and/or isolation.

Compound 112, is functionalized at the 4-position of the pyrazole ring by conventional methods to provide for compound 113. For example, when X is halo, compound 112 is contacted with at least a stoichiometric amount of a suitable halogenation agent such as N-halo succinimide, Br₂, and the like. The reaction is conducted in an inert diluent such as dimethylformamide, dichloromethane and the like at a temperature sufficient to effect halogenation. Typically, the reaction is conducted at from about 0 to about 40° C. until reaction is substantially complete which typically occurs in about 0.1 to 10 hours. The resulting product, compound 113, can be recovered by conventional methods, such as chromatography, filtration, crystallization, and the like or, alternatively, used in the next step without purification and/or isolation.

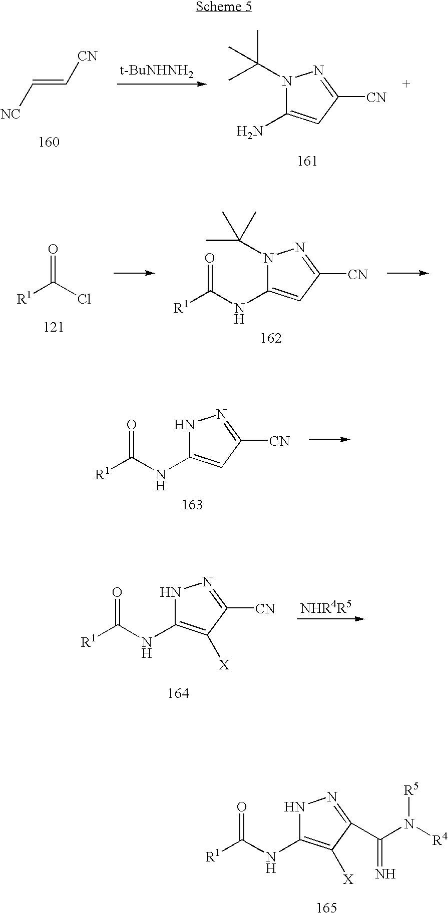

In one embodiment the substituted pyrazoles of Formula I in which Z is NH, the substituted pyrazoles are prepared as shown in Scheme 5.

Specifically, in Scheme 5, 3-amino-5-cyano pyrazole, compound 161, is prepared by the addition of tert-butylhydrazine to fumaronitrile, compound 160. This reaction is typically run at a temperature of from about 0 to about 110° C. until substantially complete, usually about 1 to about 48 hours. The resulting product, compound 161 can be recovered by conventional methods, such as chromatography, filtration, crystallization, and the like or, alternatively, used in the next step without purification and/or isolation.

The 3-amino-5-cyano pyrazole, compound 161, is acylated under conventional conditions by reaction with a desired acyl chloride, compound 121 to provide compound 162. The reaction is preferably conducted in the presence of a conventional activating agent such as DMAP in the presence of a base such as pyridine that scavenges the acid generated. The reaction is preferably conducted in an inert solvent such as dichloromethane, chloroform and the like although a liquid base such as pyridine can be employed as the solvent and to scavenge the acid generated. The resulting product, compound 163, is obtained after a standard deprotection of compound 162, and can be recovered by conventional methods, such as chromatography, filtration, crystallization, and the like or, alternatively, used in the next step without purification and/or isolation.

Compound 163, is functionalized at the 4-position of the pyrazole ring by conventional methods to provide for compound 164. For example, when X is halo, compound 163 is contacted with at least a stoichiometric amount of a suitable halogenation agent such as N-halo succinimide, bromine, and the like. The reaction is conducted in an inert diluent such as dimethylformamide, dichloromethane and the like at a temperature sufficient to effect halogenation. Typically, the reaction is conducted at from about 0 to about 40° C. until reaction is substantially complete which typically occurs in about 0.1 to 10 hours. The resulting product, compound 164, can be recovered by conventional methods, such as chromatography, filtration, crystallization, and the like or, alternatively, used in the next step without purification and/or isolation.

Compound 164 is converted to the amidine, compound 165, under conventional conditions known in the art. Formation of amidines from nitrites can be accomplished using a number of known methods including condensation with amines. Other methods of preparing amidines are illustrated by Willi Kantlehner in Comprehensive Organic Synthesis Barry M. Trost, Ed.; Pergamon Press: Oxford, 1991; Vol. 6, Chapter 2.7.

R¹ may be a sulfonated aminoalkyl such as Formula (VI) below, wherein R²¹ is hydrogen or methyl, and R²⁰ is an amino acid side chain or where R²⁰ and R²¹ and the atoms to which they are attached form a heterocyclic or heteroaryl group of from 4 to 12 ring atoms, and R²² is alkyl, substituted alkyl, aryl or substituted aryl.

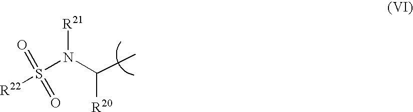

Compounds of Formula (I) or Formula (II) wherein R¹ is such a sulfonated amino group may be prepared as shown in Scheme 6 below where X, Z, Q, R², R³, R²⁰, R²¹ and R²² are as defined herein.

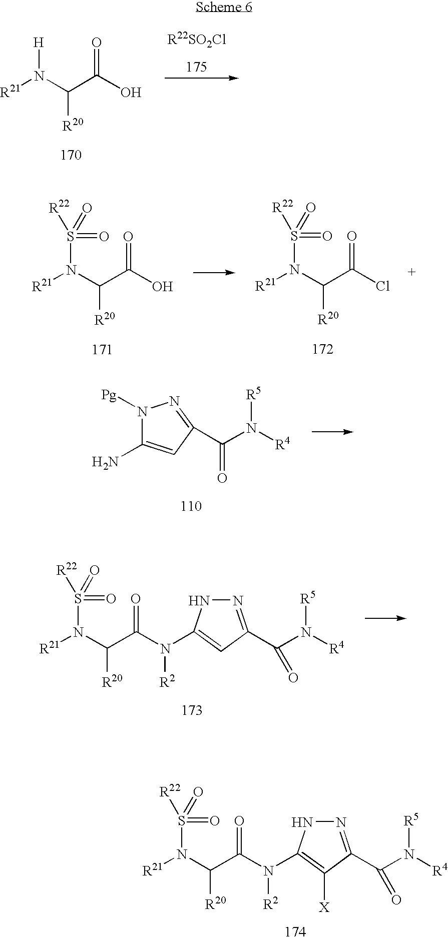

The amine group of compound 170 is converted to the sulfonamide using a suitable sulfonyl chloride, compound 175, and standard reactions conditions. For example, compound 170 may be reacted with an aryl sulfonyl chloride, compound 175, in the presence of a suitable base such as sodium carbonate an inert solvent such as as water at a temperature of about 0° C. to about 100° C. until the reaction is substantially complete, typically 1 to about 24 hours. The product, compound 171, can be recovered by conventional methods, such as chromatography, filtration, crystallization, and the like or, alternatively, used in the next step without purification and/or isolation.

Compound 171 is then converted to the acyl chloride using standard conditions. For example, compound 171 may be reacted with SOCl₂ in an inert solvent such as dichloromethane at a temperature of about −10° C. to about 39° C. until the the reaction is substantially complete, typically 1 to about 24 hours. The product, compound 172, can be recovered by conventional methods, such as filtration, crystallization, and the like or, alternatively, used in the next step without purification and/or crystallization.

Compound 172 is then coupled to compound 110 to form compound 173, using well known methods which are described herein above for the amidation reactions in Scheme 1 (used to prepare compound 124 and/or compound 125). Compound 123 is functionalized at the 4-position of the pyrazole ring by conventional methods which are described herein above for the halogenation reactions in Scheme 1 (used to prepare compound 107 and/or 125) to afford compound 174.

The sulfonyl chlorides, compound 175, employed in the above reaction are either known compounds or compounds that can be prepared from known compounds by conventional synthetic procedures. Such compounds are typically prepared from the corresponding sulfonic acid, i.e., from compounds of the formula R²²—SO₃H where R²² is as defined above, using phosphorous trichloride and phosphorous pentachloride. This reaction is generally conducted by contacting the sulfonic acid with about 2 to 5 molar equivalents of phosphorous trichloride and phosphorous pentachloride, either neat or in an inert solvent, such as dichloro-methane, at temperature in the range of about 0° C. to about 80° C. for about 1 to about 48 hours to afford the sulfonyl chloride. Alternatively, the sulfonyl chlorides can be prepared from the corresponding thiol compound, i.e., from compounds of the formula R²²—SH where R²² is as defined herein, by treating the thiol with chlorine (Cl₂) and water under conventional reaction conditions.

Pharmaceutical Formulations

When employed as pharmaceuticals, the compounds of Formula (I) or Formula (II) are usually administered in the form of pharmaceutical compositions. These compounds can be administered by a variety of routes including oral, rectal, transdermal, subcutaneous, intravenous, intramuscular, and intranasal. These compounds are effective as both injectable and oral compositions. Such compositions are prepared in a manner well known in the pharmaceutical art and comprise at least one active compound.

This invention also includes pharmaceutical compositions that contain, as the active ingredient, one or more of the compounds of Formula (I) or Formula (II) above associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound is effective over a wide dosage range and is generally administered in a pharmaceutically effective amount. It, will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices which deliver the formulation in an appropriate manner.

The following formulation examples illustrate the pharmaceutical compositions of the present invention.

### FORMULATION EXAMPLE 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### FORMULATION EXAMPLE 2

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### FORMULATION EXAMPLE 3

A dry powder inhaler formulation is prepared containing the following components:

| Ingredient | Weight % |
|---|---|
| Active Ingredient | 5 |
| Lactose | 95 |

The active mixture is mixed with the lactose and the mixture is added to a dry powder inhaling appliance.

### FORMULATION EXAMPLE 4

Tablets, each containing 30 mg of active ingredient, are prepared as follows:

| | Quantity |
|---|---|
| Ingredient | (mg/tablet) |
| Active Ingredient | 30.0 mg |
| Starch | 45.0 mg |
| Microcrystalline cellulose | 35.0 mg |
| Polyvinylpyrrolidone | 4.0 mg |
| (as 10% solution in water) | |
| Sodium carboxymethyl starch | 4.5 mg |
| Magnesium stearate | 0.5 mg |
| Talc | 1.0 mg |
| Total | 120 mg |

The active ingredient, starch and cellulose are passed through a No. 20 mesh U.S. sieve and mixed thoroughly. The solution of polyvinyl-pyrrolidone is mixed with the resultant powders, which are then passed through a 16 mesh U.S. sieve. The granules so produced are dried at 50E to 60EC and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 150 mg.

### FORMULATION EXAMPLE 5

Capsules, each containing 40 mg of medicament are made as follows:

| | |
|---|---|
| | Quantity |
| Ingredient | (mg/capsule) |

| | | |
|---|---|---|
| Active Ingredient | 40.0 | mg |
| Starch | 109.0 | mg |
| Magnesium stearate | 1.0 | mg |
| Total | 150.0 | mg |

The active ingredient, cellulose, starch, an magnesium stearate are blended, passed through a No. 20 mesh U.S. sieve, and filled into hard gelatin capsules in 150 mg quantities.

### FORMULATION EXAMPLE 6

Suppositories, each containing 25 mg of active ingredient are made as follows:

| Ingredient | Amount |
|---|---|
| Active Ingredient | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### FORMULATION EXAMPLE 7

Suspensions, each containing 50 mg of medicament per 5.0 mL dose are made as follows:

| Ingredient | Amount |
|---|---|
| Active Ingredient | 50.0 mg |
| Xanthan gum | 4.0 mg |
| Sodium carboxymethyl cellulose (11%) | |
| Microcrystalline cellulose (89%) | 50.0 mg |
| Sucrose | 1.75 mg |
| Sodium benzoate | 10.0 mg |
| Flavor and Color | q.v. |
| Purified water | to 5.0 mL |

The medicament, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### FORMULATION EXAMPLE 8

| | Quantity |
|---|---|
| Ingredient | (mg/capsule) |
| Active Ingredient | 15.0 mg |
| Starch | 407.0 mg |
| Magnesium stearate | 3.0 mg |
| Total | 425.0 mg |

The active ingredient, cellulose, starch, and magnesium stearate are blended, passed through a No. 20 mesh U.S. sieve, and filled into hard gelatin capsules in 560 mg quantities.

### FORMULATION EXAMPLE 9

An intravenous formulation may be prepared as follows:

| Ingredient | Quantity |
|---|---|
| Active Ingredient | 250.0 mg |
| Isotonic saline | 1000 mL |

### FORMULATION EXAMPLE 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, which is incorporated herein by reference in its entirety. Such patches may be constructed for continuous, pulsatile, or on demand delivery of pharmaceutical agents.

When it is desirable or necessary to introduce the pharmaceutical composition to the brain, either direct or indirect techniques may be employed. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is incorporated herein by reference in its entirety.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

Utility

Bradykinin (“BK”) is a kinin that plays an important role in the patho-physiological processes accompanying acute and chronic pain and inflammation. Bradykinins, like other related kinins, are autocoid peptides produced by the catalytic action of kallikrein enzymes on plasma and tissue precursors termed kininogens. Inhibition of bradykinin B1 receptors by compounds that are bradykinin B1 antagonists or inverse agonists would provide relief from maladies that mediate undesirable symptoms through a BK B1 receptor pathway.

The compounds of this invention are the bradykinin B₁ receptor antagonists and therefore are suitable for use in blocking or ameliorating pain as well as hyperalgesia in mammals. Such compounds would be effective in the treatment or prevention of pain including, for example, bone and joint pain (osteoarthritis), repetitive motion pain, dental pain, cancer pain, myofascial pain (muscular injury, fibromyalgia), perioperative pain (general surgery, gynecological) and chronic pain. In particular, inflammatory pain such as, for example, inflammatory airways disease (chronic obstructive pulmonary disease) would be effectively treated by bradykinin B1 antagonist compounds.

The compounds of this invention are also useful in the treatment of disease conditions in a mammal that are mediated, at least in part, by bradykinin B₁ receptor. Examples of such disease conditions include asthma, inflammatory bowel disease, rhinitis, pancreatitis, cystitis (interstitial cystitis), uveitis, inflammatory skin disorders, rheumatoid arthritis and edema resulting from trauma associated with burns, sprains or fracture. They may be used subsequent to surgical intervention (e.g. as post-operative analgesics) and to treat inflammatory pain of varied origins (e.g. osteoarthritis, rheumatoid arthritis, rheumatic disease, tenosynovitis and gout) as well as for the treatment of pain associated with angina, menstruation of cancer. They may be used to treat diabetic vasculopathy, post capillary resistance or diabetic symptoms associated with insulitis (e.g. hyperglycemia, diuresis, proteinuria and increased nitrite and kallikrein urinary excretion). They may be used as smooth muscle relaxants for the treatment of spasm of the gastrointestinal tract or uterus or in the therapy of Crohn's disease, ulcerative colitis or pancreatitis. Such compounds may be used therapeutically to treat hyperreactive airways and to treat inflammatory events associated with airways disease e.g. asthma, and to control, restrict or reverse airways hyperreactivity in asthma. They may be used to treat intrinsic and extrinsic asthma including allergic asthma (atopic or non-atopic) as well as exercise-induced asthma, occupational asthma, asthma post-bacterial infection, other non-allergic asthmas and “wheezy-infant syndrome”. They may also be effective against pneumoconiosis, including aluminosis, anthracosis, asbestosis, chalicosis, ptilosis, siderosis, silicosis, tabacosis and byssinosis was well as adult respiratory distress syndrome, chronic obstructive pulmonary or airways disease, bronchitis, allergic rhinitis, and vasomotor rhinitis. Additionally, they may be effective against liver disease, multiple sclerosis, atherosclerosis, Alzheimer's disease, septic shock e.g. as anti-hypovolemic and/or anti-hypotensive agents, cerebral edema, headache, migraine, closed head trauma, irritable bowel syndrome and nephritis. Finally, such compounds are also useful as research tools (in vivo and in vitro).

As noted above, the compounds of this invention are typically administered to the mammal in the form of a pharmaceutical composition. Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985).

In order to enhance serum half-life, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. No. 4,235,871, Geho et al, U.S. Pat. No. 4,501,728 and Allen et al., U.S. Pat. No. 4,837,028 each of which is incorporated herein by reference.

The amount administered to the patient will vary depending upon what is being administered, the purpose of the administration, such as prophylaxis or therapy, the state of the patient, the manner of administration, and the like all of which are within the skill of the attending clinician. In therapeutic applications, compositions are administered to a patient already suffering from a disease in an amount sufficient to cure or at least partially arrest the symptoms of the disease and its complications. An amount adequate to accomplish this is defined as “therapeutically effective dose.” Amounts effective for this use will depend on the disease condition being treated as well as by the judgment of the attending clinician depending upon factors such as the severity of the inflammation, the age, weight and general condition of the patient, and the like.

The compositions administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention will vary according to, for example, the particular use for which the treatment is made, the manner of administration of the compound, the health and condition of the patient, and the judgment of the prescribing physician. For example, for intravenous administration, the dose will typically be in the range of about 20 μg to about 500 μg per kilogram body weight, preferably about 100 μg to about 300 μg per kilogram body weight. Effective doses can be extrapolated from dose-response curves derived from in vitro or animal model test systems.

Alternatively, about 0.1 mg/day to about 1,000 mg/day of a compound, or mixture of compounds, of the present invention may be admistered orally, preferably from about 1 mg/day to about 100 mg/day, and more preferably from 5 mg/day to about 50 mg/day. From about 0.5 to about 100 mg/day may be given to a patient for parenteral, sublingual, intranasal or intrathecal administration; for depo administration and implants, from about 0.5 mg/day to about 50 mg/day; for topical administration from about 0.5 mg/day to about 200 mg/day; for rectal administration from about 0.5 mg to about 500 mg; and more preferably for parenteral administration, from about 5 to about 50 mg daily.

The following synthetic and biological examples are offered to illustrate this invention and are not to be construed in any way as limiting the scope of this invention. Unless otherwise stated, all temperatures are in degrees Celsius.

### EXAMPLES

In the examples below, the following abbreviations have the following meanings. If an abbreviation is not defined, it has its generally accepted meaning.
Ac=Acetyl
Boc=t-butoxycarbonyl
BOP=Benzotriazol-1-yloxytris(dimethylamino)-phosphonium hexafluorophosphate
bs=broad singlet
conc.=concentrated
d=doublet
dd=doublet of doublets
DIAD=diisopropyl azodicarboxylate
DEEA=diisopropylethyl amine
DMAP=4-N,N-dimethylaminopyridine
DMF=N,N-dimethylformamide
EDC or EDC.HCL=1-(3-dimethylaminopropyl)-3-ethylcarbodiimide hydrochloride
Et=ethyl
EtOH=ethanol
eq.=equivalents
g=gram
h=hours
HATU=O-(7-azabenzotriazol-1-yl)-1,1,3-,3-tetramethyl-uronium hexafluorophosphate
HOAc=acetic acid
HOBT=1-hydroxybenzothiazole hydrate
Hz=hertz
HPLC=high performance liquid chromatography
MS=mass spectroscopy
Me=methyl
MeOH=methanol
m=multiplet
M=molar
mg=milligram
min.=minutes
mL=milliliter
MHz=Megahertz
mmol=millimolar
NBSN=N-bromosuccinamide
NCS=N-chlorosuccinamide
NMR=nuclear magnetic resonance
NMM=N-methylmorpholine
OAc=acetate
psi=pounds per square inch
q=quartet
rt=room temperature
Rt=retention time
s=singlet
t=triplet
THF=tetrahydrofuran
μL=microliters
δ=chemical shift
DMSO=dimethyl sulfonamide
EtOAc=ethyl acetate
J=coupling constant
p-Tos-OH=para toluene sulfonic acid
TFA=trifluoroacetic acid

In the following examples and procedures, the term “Aldrich” indicates that the compound or reagent used in the procedure is commercially available from Aldrich Chemical Company, Inc., Milwaukee, Wis. 53233 USA; the term “Sigma” indicates that the compound or reagent is commercially available from Sigma, St. Louis Mo. 63178 USA; the term “TCI” indicates that the compound or reagent is commercially available from TCI America, Portland Oreg. 97203; the term “Frontier” or “Frontier Scientific” indicates that the compound or reagent is commercially available from Frontier Scientific, Utah, USA; the term “Bachem” indicates that the compound or reagent is commercially available from Bachem, Torrance, Calif., USA. The term “Matrix” indicates that the compound or reagent is commercially available from Matrix Scientific, Columbia, S.C., USA. The term “Ambinter” indicates that the compound or reagent is commercially available from Ambinter Paris, France. The term “Novabiochem” indicates that the compound or reagent is commercially available from EMD, Biosciences, Inc, San Diego, Calif.

The following general procedures illustrate general synthetic pathways for preparing 3-amido-5-substituted pyrazole derivatives of Formula (I) or Formula (II) and amine intermediates useful in preparing the same.

### General Procedure 1

### Preparation of tert-Butoxycarbonylaminoacetyl amides (3)

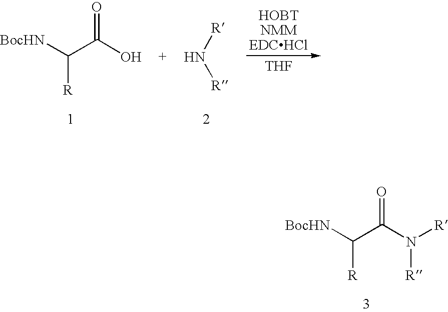

A mixture of 1.0 eq. of Boc-protected amino acid 1, 1.2 eq. of amine (2), 1.2 eq. of HOBT, 2.2 eq. of NMM, and 1.2 eq. of EDC•HCl in THF was stirred at rt. After a time sufficient for reaction completion, 1 M HCl was added to the reaction mixture. The acidified solution was extracted with EtOAc. The combined organic extracts were washed with saturated aqueous NaHCO₃ followed by drying over MgSO₄. The mixture was filtered. The filtrate was rotary evaporated and dried under vacuum to give amide 3.

### General Procedure 2

### Preparation of 4-Bromo-5-(2-chlorobenzoylamino)-1H-pyrazole-3-carboxylic acid amides

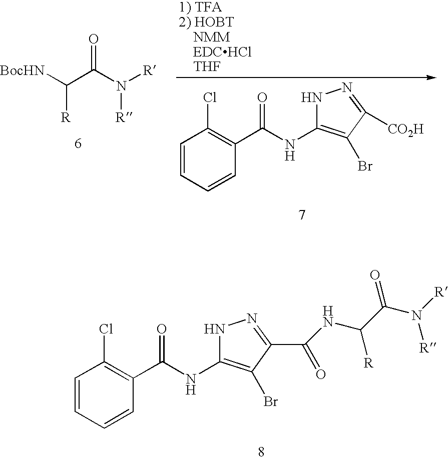

Amide 6 was prepared as described in General Procedure 1. Compound 8 was prepared as shown in General Procedure 2. A solution of 1.2 eq. of Boc-protected amide 6 in neat TFA was stirred at rt for 30 min. The reaction solution was rotary evaporated and dried under vacuum to afford the free amine. The deprotected amine was dissolved in THF. While stirring, 1.0 eq. of 7, 1.3 eq. of HOBT, 2.2 eq. of NMM, and 1.2 eq. of EDC.HCl were added in that order. After stirring the reaction mixture at rt for a time sufficient for reaction completion, the mixture was rotary evaporated. The crude material was dissolved in MeOH and flash chromatographed using a mixture of EtOAc-hexanes as eluant to afford product 8.

### General Procedure 3

### Preparation of [4-Bromo-5-(2-chlorobenzoylamino)-1H-pyrazole-3-carbonyl]aminoacetic acids (10)

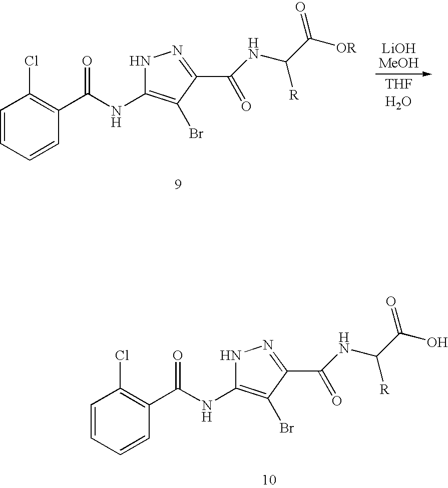

A solution of 1.0 eq. of ester 9 and 2.6 eq. of LiOH.H₂O in MeOH, THF, and H₂O (1:2:1) was stirred at rt. After a time sufficient for reaction completion, the reaction solution was rotary evaporated. The crude material was dissolved in water and 1 M HCl was added until the solution's pH was about 4. The acidified solution was extracted with EtOAc. The combined organic extracts were dried over MgSO₄ and filtered. The filtrate was rotary evaporated. The crude product was purified by crystallization from MeOH and hexanes to yield acid 10.

### General Procedure 5

### Preparation of 4-Bromo-5-(2-chlorobenzoylamino)-1H-pyrazole-3-carboxylic acid (7)

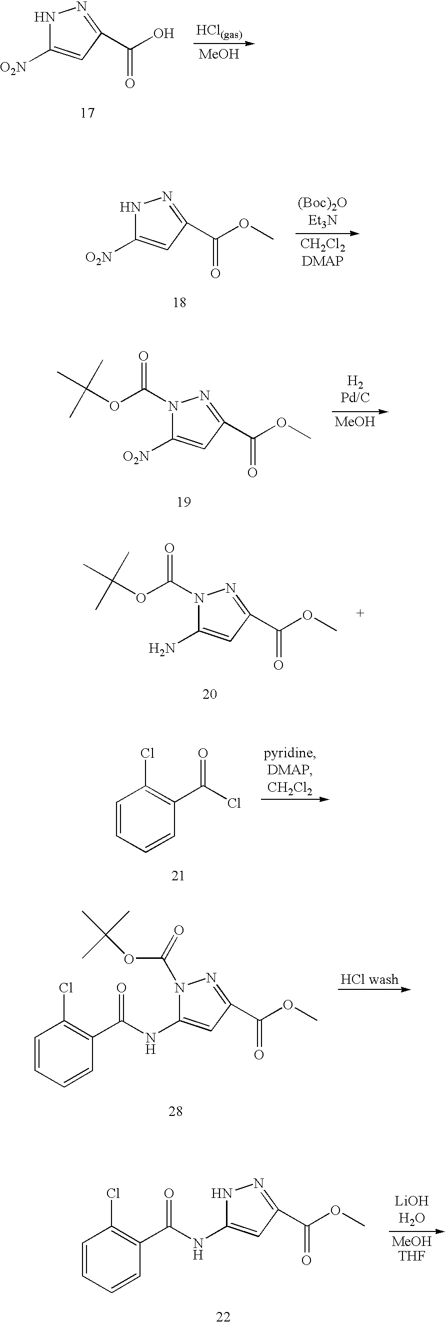

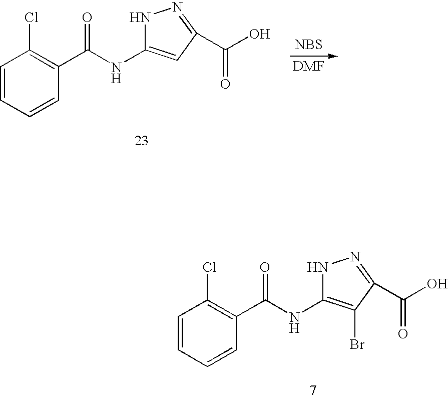

Preparation of 3-Methoxycarbonyl-5-nitropyrazole (18). A solution of 5-nitro-1H-pyrazole-3-carboxylic acid (17, Aldrich, cat. no. 41,483-2) in MeOH was prepared. While stirring, HCl was bubbled through the solution for 2 min. The reaction mixture was refluxed for a time sufficient for complete esterification and allowed to cool to rt. The solvent was removed by rotary evaporation. The crude material was basified by addition of saturated aqueous NaHCO₃ and extracting with EtOAc. The combined organic extracts were dried over MgSO₄ and filtered. The filtrate was rotary evaporated and dried under vacuum to yield 18.

Preparation of 1-tert-Butoxycarbonyl-3-methoxycarbonyl-5-nitropyrazole (19). A solution of 1.0 eq. of 18, 1.1 eq. of (Boc)₂O, 1.5 eq. of Et₃N, and 0.05 eq. of DMAP in CH₂Cl₂ was prepared. The reaction mixture was stirred at rt for a time sufficient for reaction completion and the solvent was removed by rotary evaporation. The crude material was dried under vacuum to afford product 19.

Preparation of 5-Amino-1-tert-butoxycarbonyl-3-methoxycarbonylpyrazole (20). A mixture of 1.0 eq. of 19 and 0.1 wt/wt eq. of 10% Pd on carbon was hydrogenated at 10-60 psi of hydrogen for a time sufficient for reaction completion. The reaction mixture was filtered through Celite. The filtrate was concentrated by rotary evaporation. The crude material was dried under vacuum to give product 20.

Preparation of 5-(2-Chloro-benzoylamino)-pyrazole-1,3-dicarboxylic acid 1-tert-butyl ester 3-methyl ester (28). A solution of 1.0 eq. of 20, 1.5 eq. of pyridine, and 0.04 eq. of DMAP in CH₂Cl₂ was prepared. After cooling to 0° C., 1.1 eq. of 2-chlorobenzoyl chloride (Aldrich, cat. no. 10,391-8) was added. The reaction solution was allowed to warm to rt and after a time sufficient for reaction completion, the solvent was removed by rotary evaporation to afford crude 28.

Preparation of 5-(2-Chlorobenzoylamino)-3-methoxycarbonylpyrazole (22). A solution of 28 in 1 M HCl was stirred for 5 min and extracted with EtOAc. The combined organic extracts were washed with saturated aqueous NaHCO₃, followed by drying over MgSO₄ and filtering. The filtrate was rotary evaporated and dried under vacuum to yield 22.

Preparation of 5-(2-Chlorobenzoylamino)-1H-pyrazole-3-carboxylic acid (23). A solution of 1.0 eq. of 22 and 5.0 eq. of LiOH.H₂O in THF, MeOH, and H₂O (2:1:1) was stirred at rt. After a time sufficient for reaction completion, the reaction mixture was rotary evaporated. The mixture was acidified with concentrated HCl. As the pH of the solution reached about 2, a precipitate formed. The solid was collected by filtration and after drying under vacuum, product 23 was obtained.

Preparation of the title compound (7). A solution of 1.0 eq. of 23 in DMF was prepared. While stirring, a solution of 1.2 eq. of N-bromosuccinamide in DMF was added. After stirring at rt for a time sufficient for reaction completion, H₂O was added. The mixture was extracted with EtOAc. The combined organic extracts were washed with 1 M HCl, followed by drying over MgSO₄ and filtering. The filtrate was rotary evaporated. The crude material was triturated with CH₂Cl₂ and dried under vacuum to yield 7.

### General Procedure 6

### Preparation of 4-Chloro-5-(2-chlorobenzoylamino)-1H-pyrazole-3-carboxylic acid (24)

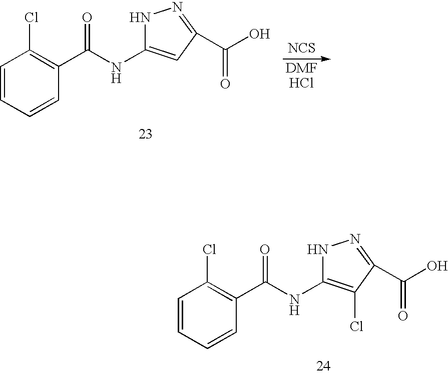

A solution of 1.0 eq. of 23 in DMF was prepared. While stirring, 1.3 eq. of N-chlorosuccinamide and a small amount of concentrated HCl were added. After stirring at rt for a time sufficient for reaction completion, H₂O was added. The quenched reaction solution was extracted with EtOAc. The combined organic extracts were dried over MgSO₄ and filtered. The filtrate was rotary evaporated. The crude material was triturated with CH₂Cl₂ and dried under vacuum to yield 24.

### General Procedure 7

### Preparation of (R)-4-Bromo-5-(2-chlorobenzoylamino)-1H-pyrazole-3-carboxylic acid amides (25)

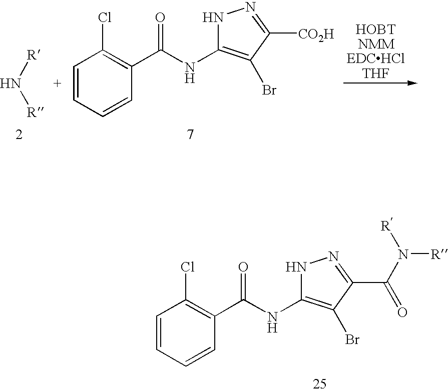

Compound 7 was prepared as shown in General Procedure 5. Compound 25 was prepared as shown in General Procedure 7. A mixture of 1.0 eq. of 7, 1.1 eq. of 2, 1.2 eq. of HOBT, 2.2 eq. of NMM, and 1.2 eq. of EDC•HCl in THF was stirred at rt. After a time sufficient for reaction completion, the reaction mixture was adsorbed onto silica gel and flash chromatographed using a mixture of EtOAc/hexanes as eluant to give product 25.

### General Procedure 8

### Preparation of 5-[(2-Chloro-benzoyl)-methyl-amino]-1H-pyrazole-3-carboxylic acid methyl ester (29)

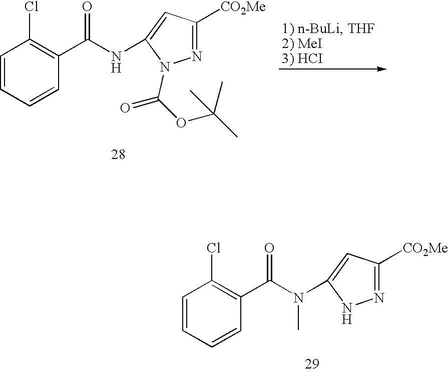

A suspension of 1.0 eq. of ester 28 (as prepared in General Procedure 5) in THF was stirred at −78° C. as 2.0 eq. of a 2.5 M solution of n-BuLi in THF was added. The cooling bath was removed and the reaction mixture was allowed to stir while warming for 10 min. The mixture was cooled back to −78° C. and 2.0 eq. of MeI was added. The bath was again removed and the reaction mixture was allowed to warm to rt. After a time sufficient for reaction completion, the reaction was quenched with 1 M HCl and extracted with EtOAc. The organic layer was washed with sat. aq. NaHCO₃, dried over MgSO₄, filtered and the solvent removed by rotary evaporation. The material was purified by flash chromatography on silica gel using a mixture of EtOAc-hexanes as eluant to afford 29.

### General Procedure 9

### Preparation of 4-Bromo-5-[2-(quinolin-8-ylthiomethyl)benzoylamino]-1H-pyrazole-3-carboxylic acid (43)

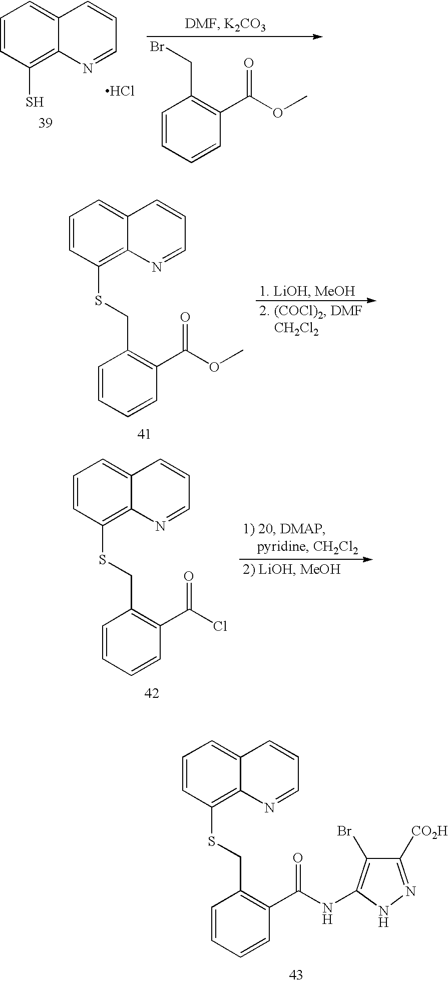

Preparation of 2-(Quinolin-8-ylthiomethyl)benzoic acid methyl ester (41). A solution of 4.0 eq. of quinoline-8-thiol hydrochloride (39, Aldrich, cat. no. 35,978-5) was dissolved in DMF. To this was added 32.0 eq. of potassium carbonate. The mixture was stirred at room temperature for 20 minutes and 1.0 eq of 2-bromomethyl-benzoic acid methyl ester (40, J. Org. Chem, 2002, 67, 2160) was added. The mixture was stirred at room temperature for a time sufficient enough for reaction completion. The mixture was diluted with 0.1 M citric acid and extracted with EtOAc. The organic layer was washed with brine and dried with Na₂SO₄, filtered, and concentrated. The product was purified by flash chromatography on silica gel using a mixture of EtOAc-hexanes as eluant to give 41.

Preparation of 2-(Quinolin-8-ylthiomethyl)benzoyl chloride (42). A solution of 1.0 eq of ester 41 and 3.0 eq. of LiOH in methanol and water was heated to 65° C. for a time sufficient for completion of the hydrolysis. The mixture was cooled to room temperature and concentrated, then diluted with H₂O. The pH of the aqueous mixture was adjusted to 4.5 and extracted with EtOAc. The organic layer was washed with brine and dried over Na₂SO₄, filtered, and concentrated to give the intermediate benzoic acid.

A solution of 1.0 eq. of 2-(quinolin-8-ylthiomethyl)benzoic acid in CH₂Cl₂ was cooled to 0° C. To this was added 1.1 eq. of oxalyl chloride followed by one drop of DMF. The mixture was warmed to room temperature and stirred for a time sufficient for reaction completion. The mixture was concentrated to give 42 which was used directly.

Preparation of the title compound (43). The procedure described for compound 22 was employed using 2-(quinolin-8-ylthiomethyl)benzoyl chloride (42). Hydrolysis of the methyl ester as described for compound 23 afforded acid 43.

### General Procedure 10

### Preparation of 4-Bromo-5-(2-chlorobenzoylamino)-1H-pyrazole-3-carboxylic acid amides (44)

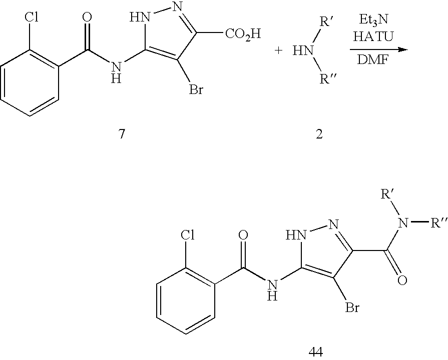

A solution of 1.0 eq. of acid 7, 1.0 eq. of amine 2, and 8.1 eq. of Et₃N in DMF was prepared. While stirring, a solution of 1.0 eq. of HATU dissolved in DMF was added. After stirring at rt for a time sufficient for reaction completion, 6.0 eq. of MP-carbonate resin and 6.0 eq. of PS-trisamine resin (both from Argonaut Technologies, Inc.) were added. The mixture was stirred at rt for 16 hrs, filtered, and washed with DMF and MeOH. The crude material was purified by reverse-phase HPLC using a mixture of acetonitrile-water as eluant. The purified material was concentrated and dried to afford amide 44.

### General Procedure 11

### Preparation of 5-(2-Chloro-benzoylamino)-4-methyl-1H-pyrazole-3-carboxylic acid (51)

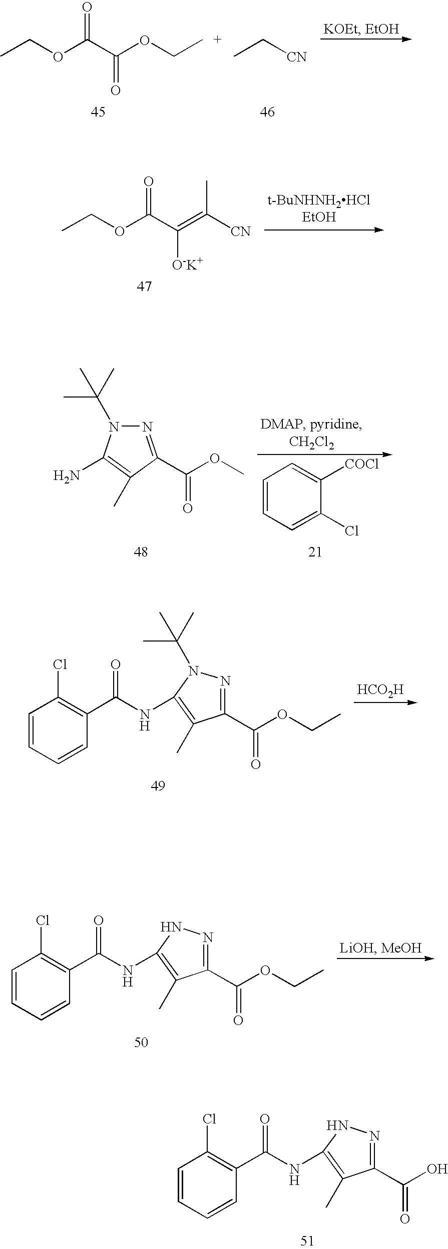

Preparation of Potassium 2-cyano-1-ethoxycarbonyl-2-methylethenolate (47). Potassium ethoxide (1.0 eq.) was placed in a sealed tube with EtOH and shaken until dissolved. A mixture of diethyl oxalate (1.0 eq.) and propionitrile (1.0 eq.) was added to the sealed tube and the mixture was capped and stirred at reflux. After a time sufficient for reaction completion, the reaction was cooled and the precipitate collected and washed with diethyl ether to afford 47.

Preparation of 5-Amino-1-tert-butyl-4-methyl-1H-pyrazole-3-carboxylic acid ethyl ester (48). 47 (1.0 eq.) was placed into a sealed pressure reaction flask followed by EtOH and t-butylhydrazine hydrochloride (1.1 eq.). The pressure flask was capped and heated to reflux. After a time sufficient for reaction completion, the mixture was evaporated to dryness and the solid obtained was dissolved in equal amounts of EtOAc and water. The organic layer was washed with saturated aqueous NaHCO₃, brine, dried with MgSO₄, and evaporated. This slightly yellow solid was triturated with hexanes and filtered to afford ester 48.

Preparation of 1-tert-Butyl-5-(2-chloro-benzoylamino)-4-methyl-1H-pyrazole-3-carboxylic acid ethyl ester (49). The procedure described for compound 22 was employed using 5-amino-1-tert-butyl-4-methyl-1H-pyrazole-3-carboxylic acid ethyl ester (49).

Preparation of 5-(2-Chloro-benzoylamino)-4-methyl-1H-pyrazole-3-carboxylic acid ethyl ester (50). Ester 49 was dissolved in a minimal amount of formic acid and heated to 80° C. for a time sufficient for reaction completion. Formic acid was removed via rotary evaporation to yield 50.

Preparation of the title compound (51). The procedure described for compound 23 was employed using 5-(2-chloro-benzoylamino)-4-methyl-1H-pyrazole-3-carboxylic acid ethyl ester 50. 51 was recovered by conventional techniques

### General Procedure 12

### Preparation of N-(5-Carboxyalkyl-4-methyl-2H-pyrazol-3-yl)-2-chloro-benzamide (55)

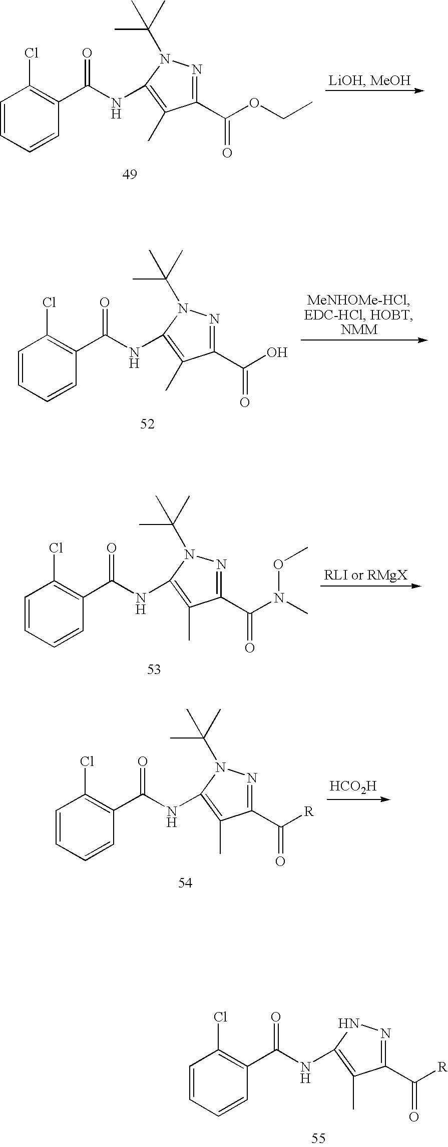

Preparation of 1-tert-Butyl-5-(2-chloro-benzoylamino)-4-methyl-1H-pyrazole-3-carboxylic acid (52). Hydrolysis as described in Procedure 3 was employed using 1-tert-butyl-5-(2-chlorobenzoylamino)-4-methyl-1H-pyrazole-3-carboxylic acid ethyl ester (49) to afford acid 52.

Preparation of 1-tert-Butyl-5-(2-chlorobenzoylamino)-4-methyl-1H-pyrazole-3-carboxylic acid methoxymethylamide (53). The procedure described for compound 3 was employed using 1-tert-butyl-5-(2-chloro-benzoylamino)-4-methyl-1H-pyrazole-3-carboxylic acid (52) and N,O-dimethylhydroxylamine hydrochloride (Aldrich, cat. no. D16,370-8).

Preparation of N-(5-Carboxyalkyl-2-tert-butyl-4-methyl-2H-pyrazol-3-yl)-2-chlorobenzamides (54). To a flask equipped with a stirbar was added 53 (1.0 eq.) dissolved in THF under a nitrogen atmosphere. The mixture was cooled to −10° C. and a 1.4 M solution of RLi, such as MeLi (6.0 eq.) in diethyl ether, was added dropwise. The mixture was allowed to slowly warm to room temperature and stirred for a time sufficient for reaction completion. The reaction was poured into 0.1 N HCl and extracted with dichloromethane, dried over MgSO₄, filtered and concentrated to a crude oil. The crude material was purified by column chromatography eluting with a mixture of EtOAc-hexanes to afford 54.

Preparation of the title compound (55). The procedure described for compound 50 in General Procedure 11 was employed using benzamide 54.

### General Procedure 13

### Preparation of 4-Bromo-5-(2-m-tolylthiomethylbenzoylamino)-1H-pyrazole-3-carboxylic acid (62)

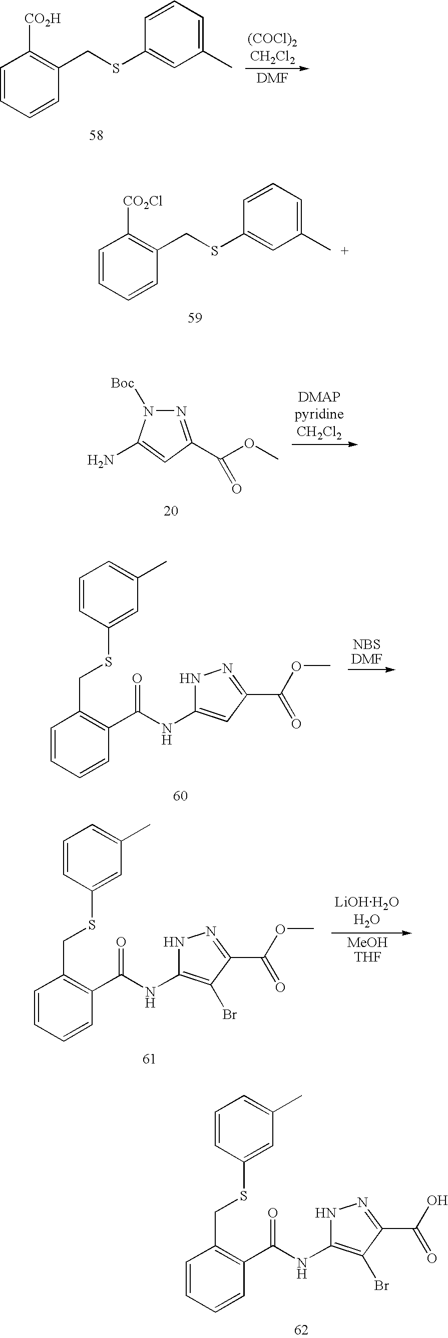

Preparation of 2-m-Tolylthiomethyl-benzoyl chloride (59). A solution of 1.0 eq. of 58 (Coll. Czech. Chem. Comm. 1982, 47, 3094) in CH₂Cl₂ was prepared. While stirring, 1.1 eq. of oxalyl chloride and one drop of DMF was added. After stirring at rt for a time sufficient for reaction completion, the reaction mixture was rotary evaporated and dried under vacuum to produce 59.

Preparation of 5-(2-m-Tolylthiomethybenzoylamino)-1H-pyrazole-3-carboxylic acid methyl ester (60). A solution of 1.1 eq. of 20, 1.1 eq. of pyridine, and 0.07 eq. of DMAP in CH₂Cl₂ was cooled to 0° C. While stirring, a solution of 1.0 eq. of 59 in CH₂Cl₂ was added. The reaction solution was allowed to warm to rt. After a time sufficient for reaction completion, the reaction solution was concentrated by rotary evaporation and 1.0 M HCl was added. The acidified solution was extracted with EtOAc. The combined organic extracts were washed with saturated aqueous NaHCO₃, followed by drying over MgSO₄ and filtering. The filtrate was rotary evaporated and the crude material was purified by flash chromatography on silica gel using a mixture of EtOAc-hexanes as eluant to give 60.

Preparation of 4-Bromo-5-(2-m-tolylthiomethylbenzoylamino)-1H-pyrazole-3-carboxylic acid methyl ester (61). A solution of 1.0 eq. of 60 in DMF was prepared. While stirring, a solution of 1.1 eq. of NBS in DMF was added. After stirring at rt for a time sufficient for reaction completion, water was added. The solution was extracted with EtOAc. The combined organic extracts dried over MgSO₄ and vacuum filtered. The filtrate was rotary evaporated and the crude material was purified by flash chromatography on silica gel using a mixture of EtOAc-hexanes as eluant to give 61.

Preparation of the title compound (62). The procedure described for compound 23 was employed with methyl ester 61 to afford acid 62.

### Example 1

### Preparation of (R)-4-Bromo-5-(2-chloro-benzoylamino-1H-pyrazole-3-carboxylic acid (1-(N,N-dimethylaminocarbonyl)eth-1-yl)amide

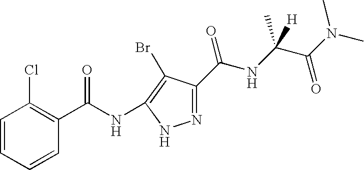

The pyrazole acid, prepared as described in Procedure 5, was coupled with (2R)-2-amino-N,N-dimethylpropanamide (prepared as described in Procedure 1) using the method described in Procedure 2.

MS+=442.0 ¹H-NMR (CDCl₃) δ 12.22 (br, 1H), 9.04 (s, 1H), 8.04 (m, 2H), 7.52-7.43 (m, 3H), 5.12 (m, 1H), 3.14 (s, 3H), 3.01 (s, 3H), 1.44 (d, J=6.8 Hz, 3H).

### Example 2

### Preparation of (S)-4-Bromo-5-(2-chloro-benzoylamino)-1H-pyrazole-3-carboxylic acid (1-(N,N-dimethylaminocarbonyl)eth-1-yl)amide

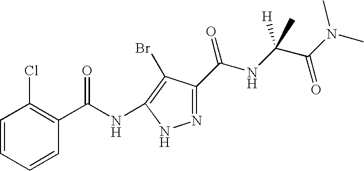

The pyrazole acid, prepared as described in Procedure 5, was coupled with (2S)-2-amino-N,N-dimethylpropanamide (prepared as described in Procedure 1) using the method described in Procedure 2.

MS+=442.0 ¹H-NMR (CDCl₃) δ 12.37 (br, 1H), 9.06 (s, 1H), 8.07 (m, 2H), 7.52-7.43 (m, 3H), 5.12 (m, 1H), 3.14 (s, 3H), 3.01 (s, 3H), 1.44 (d, J=6.8 Hz, 3H).

### Example 3

### Preparation of (S)-4-Bromo-5-(2-chloro-benzoylamino)-1H-pyrazole-3-carboxylic acid [1-(phenyl)-1-(methoxycarbonyl)methyl]amide

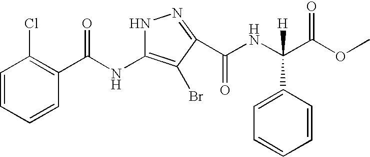

The pyrazole acid, prepared as described in Procedure 5, was coupled with (S)-(+)-2-phenylglycine methyl ester hydrochloride (Aldrich, 30,867-6) using the method described in Procedure 7.

MS+=491.0 ¹H-NMR (CDCl₃) δ 9.21 (br, 1H), 8.12 (br, 1H), 7.97, (br, 1H), 7.45 (m, 5H), 7.30 (m, 4H), 5.78 (m, 1H), 3.71 (s, 3H).

### Example 4

### Preparation of (S)-4-Bromo-5-(2-chloro-benzoylamino)-1H-pyrazole-3-carboxylic acid [1-(phenyl)-1-(carboxy)methyl]amide

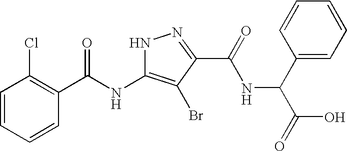

The pyrazole acid, prepared as described in Procedure 5, was coupled with L-phenylglycine methyl ester (Novabiochem, 04-10-0034) using the method described in Procedure 7 followed by hydrolysis using the method described in Procedure 3.

MS+=478.9 ¹H-NMR (CD₃OD) δ 7.66 (m, 1H), 7.52 (m, 5H), 7.35 (m, 3H), 5.52 (s, 1H).

### Example 5

### Preparation of (S)-5-(2-chloro-benzoylamino)-1H-pyrazole-3-carboxylic acid [1-(phenyl)-1-(methoxycarbonyl)methyl]amide

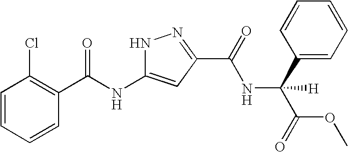

The pyrazole acid, prepared as described in Procedure 5, was coupled with (S)-(+)-2-phenylglycine methyl ester hydrochloride (Aldrich, 30,867-6) using the method described in Procedure 7.

MS+=413.0 ¹H-NMR (CDCl₃) δ 8.60 (br, 1H), 7.70 (m, 1H), 7.41 (m, 5H), 7.15 (m, 2H), 5.44 (m, 1H), 3.98 (s, 3H).

### Example 6

### Preparation of (S)-4-Bromo-5-(2-chloro-benzoylamino)-1H-pyrazole-3-carboxylic acid [2-(phenyl)-1-(methoxycarbonyl)eth-1-yl)]amide

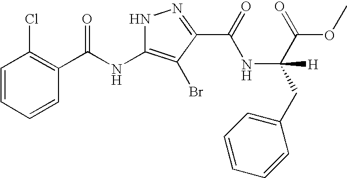

The pyrazole acid, prepared as described in Procedure 5, was coupled with L-phenylalanine methyl ester hydrochloride, (Bachem, E-2270) using the method described in Procedure 7.

MS+=505.0 ¹H-NMR (CDCl₃) δ 12.20 (br, 1H), 9.09 (br, 1H), 8.04 (m, 1H), 7.50 (m, 4H), 7.26 (m, 5H), 5.09 (m, 1H), 3.75 (s, 3H), 3.25 (m, 2H), 1.62 (s, 2H).

### Example 7

### Preparation of (R)-4-Bromo-5-(2-chloro-benzoylamino)-1H-pyrazole-3-carboxylic acid [1-(phenyl)-1-(methoxycarbonyl)methyl]amide

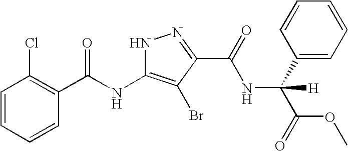

The pyrazole acid, prepared as described in Procedure 5, was coupled with (R)-(−)-2-phenylglycine methyl ester hydrochloride, (Aldrich, 30,788-2) using the method described in Procedure 7.

MS+=490.9 ¹H-NMR (CDCl₃) δ 12.38 (br, 1H), 9.13 (br, 1H), 8.01 (m, 2H), 7.41 (m, 8H), 5.78 (m, 1H), 3.74 (s, 3H).

### Example 8

### Preparation of (S)-4-Bromo-5-(2-m-tolylthiomethyl-benzoylamino)-1H-pyrazole-3-carboxylic acid [1-(phenyl)-1-(methoxycarbonyl)methyl]amide

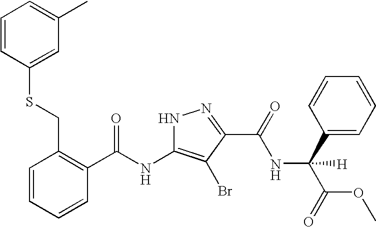

The pyrazole acid, prepared as described in Procedure 13, was coupled with (S)-(+)-2-phenylglycine methyl ester hydrochloride, (Aldrich, 30,867-6) using the method described in Procedure 7.

MS+=593.0 ¹H-NMR (CDCl₃) δ 8.55 (s, 1H), 8.04 (d, J=7.7 Hz, 1H), 7.57 (m, 1H), 7.37 (m, 8H), 7.07 (m, 3H), 6.98 (m, 1H), 5.78 (m, 1H), 4.34 (s, 2H), 3.73 (s, 3H), 2.20 (s, 3H).

### Example 9

### Preparation of (S)-2-{[4-Bromo-5-(2-chloro-benzoylamino)-1H-pyrazole-3-carboxylic acid [1-(methoxycarbonyl)pent-1-yl]amide

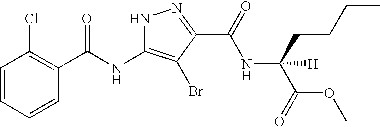

The pyrazole acid, prepared as described in Procedure 5, was coupled with L-norleucine methyl ester hydrochloride (Bachem, F-1930) using the method described in Procedure 7.

MS+=471.0 ¹H-NMR (CDCl₃) δ 9.11 (br, 1H), 8.03 (d, J=7.1 Hz, 1H), 7.61 (d, J=8.8 Hz, 1H), 7.49 (m, 3H), 4.78 (m, 1H), 3.76 (s, 3H), 1.86 (m, 2H), 1.34 (m, 4H), 0.87 (t, J=6.6 Hz, 3H).

### Example 10

### Preparation of (S)-4-Bromo-5-(2-chloro-benzoylamino)-1H-pyrazole-3-carboxylic acid [1-(phenyl)-1-(methylaminocarbonyl)methyl]amide

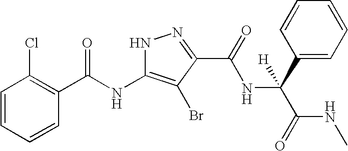

The pyrazole acid, prepared as described in Procedure 5, was coupled with Boc-D-phenylglycine methylamide (prepared as described in Procedure 1) using the method described in Procedure 2.

MS+=489.9 ¹H-NMR (CDCl₃) δ 9.11 (br, 1H), 8.43 (d, J=7.7 Hz, 1H), 7.96 (d, J=7.7 Hz, 1H), 7.38 (m, 5H), 7.26 (m, 3H), 6.74 (br, 1H), 2.74 (m, 3H).

### Example 11

### Preparation of (S)-4-Bromo-5-(2-chloro-benzoylamino)-1H-pyrazole-3-carboxylic acid [1-(piperidin-1-ylcarbonyl)eth-1-yl]amide

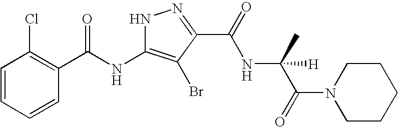

The pyrazole acid, prepared as described in Procedure 5, was coupled with Boc-(S)-(1-methyl-2-oxo-2-piperidin-1-yl-ethyl)-amine (prepared as described in Procedure 1) using the method of Procedure 2.

MS+=482.0 ¹H-NMR (CDCl₃) δ 9.21 (br, 1H), 8.19 (d, J=7.7 Hz, 1H), 7.95 (d, J=7.1 Hz, 1H), 7.41 (m, 3H), 5.06 (m, 1H), 3.3.54 (m, 4H), 1.58 (m, 6H), 1.37 (d, J=6.6 Hz, 3H).

### Example 12

### Preparation of 4-Bromo-5-(2-chloro-benzoylamino)-1H-pyrazole-3-carboxylic acid [1-(N-morpholinocarbonyl)eth-1-yl]amide

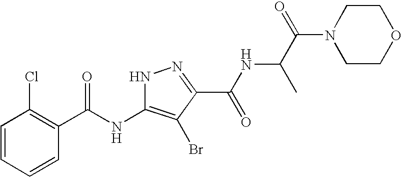

The pyrazole acid, prepared as described in Procedure 5, was coupled with Boc-(1-methyl-2-morpholin-4-yl-2-oxo-ethyl)-amine (prepared as described in Procedure 1) using the method described in Procedure 2.

MS+=484.0 ¹H-NMR (CDCl₃) δ 9.18 (br, 1H), 8.14 (d, J=7.7 Hz, 1H), 8.00 (d, J=7.1 Hz, 1H), 7.43 (m, 3H), 5.10 (m, 1H), 3.62 (m, 8H), 1.40 (d, J=6.6 Hz, 3H).

### Example 13

### Preparation of (R)-4-Bromo-5-(2-chloro-benzoylamino)-1H-pyrazole-3-carboxylic acid [1-(piperidin-1-ylcarbonyl)eth-1-yl]amide

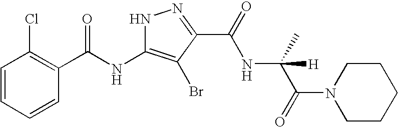

The pyrazole acid, prepared as described in Procedure 5, was coupled with Boc-(R)-(1-methyl-2-oxo-2-piperidin-1-yl-ethyl)-amine (prepared as described in Procedure 1) using the method of Procedure 2.

MS+=482.0 ¹H-NMR (CDCl₃) δ 9.20 (s, 1H), 8.19 (d, J=7.7 Hz, 1H), 7.95 (d, J=7.1 Hz, 1H), 7.41 (m, 3H), 5.05 (m, 1H), 3.50 (m, 4H), 1.53 (m, 5H), 1.38 (d, J=6.6 Hz, 3H).

### Example 14

### Preparation of (S)-4-Bromo-5-(2-chloro-benzoylamino)-1H-pyrazole-3-carboxylic acid [1-(phenyl)-1-(-piperidin-1-ylcarbonyl)methyl]amide

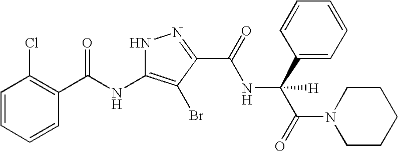

The pyrazole acid, prepared as described in Procedure 5, was coupled with Boc-(S)-(2-oxo-1-phenyl-2-piperidin-1-yl-ethyl)-amine (prepared as described in Procedure 1) using the method of Procedure 2.

MS+=544.1 ¹H-NMR (CDCl₃) δ 9.08 (br, 1H), 8.53 (d, J=7.7 Hz, 1H), 7.96 (d, J=7.1 Hz, 1H), 7.39 (m, 8H), 6.01 (m, 1H), 3.74 (m, 1H), 3.47 (m, 1H), 3.33 (m, 2H), 1.90 (m, 1H), 1.46 (m, 4H), 0.97 (m, 1H).

### Example 15

### Preparation of (R)-4-Bromo-5-(2-chloro-benzoylamino)-1H-pyrazole-3-carboxylic acid [1-(N-morpholinocarbonyl)eth-1-yl]amide

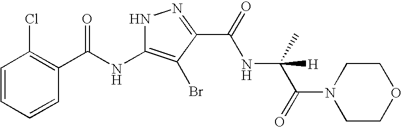

The pyrazole acid, prepared as described in Procedure 5, was coupled with Boc-(R)-(1-methyl-2-morpholin-4-yl-2-oxo-ethyl)-amine (prepared as described in Procedure 1) using the method described in Procedure 2.

MS+=484.0 ¹H-NMR (CDCl₃) δ 9.21 (br, 1H), 8.22 (m, 1H), 7.97 (m, 1H), 7.43 (m, 3H), 5.12 (m, 1H), 3.55 (m, 8H), 1.41 (d, J=9.0, 3H).

### Example 16

### Preparation of (R)-4-Bromo-5-(2-chloro-benzoylamino)-1H-pyrazole-3-carboxylic acid [1-(phenyl)-1-(piperidin-1-ylcarbonyl)methyl]amide

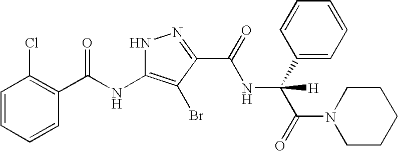

The pyrazole acid, prepared as described in Procedure 5, was coupled with Boc-(R)-(2-oxo-1-phenyl-2-piperidin-1-yl-ethyl)-amine (prepared as described in Procedure 1) using the method of Procedure 2.

MS+=544.0 ¹H-NMR (CDCl₃) δ 9.11 (br, 1H), 8.54 (d, J=7.1 Hz, 1H), 7.94 (d, J=7.1 Hz, 1H), 7.37 (m, 8H), 6.02 (m, 1H), 3.74 (m, 1H), 3.40 (m, 3H), 1.36 (m, 6H).

### Example 17

### Preparation of (R)-5-(2-Chloro-benzoylamino)-1H-pyrazole-3-carboxylic acid [1-(piperidin-1-ylcarbonyl)eth-1-yl]amide

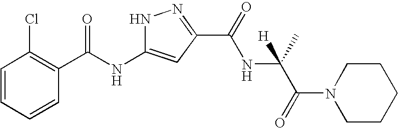

The pyrazole acid, prepared as described in Procedure 5, was coupled with Boc-(R)-(1-methyl-2-oxo-2-piperidin-1-yl-ethyl)-armine (prepared as described in Procedure 1) using the method of Procedure 2.

MS+=404.1 ¹H-NMR (CDCl₃) δ 7.76 (d, J=7.1 Hz, 1H), 7.63 (m, 1H), 7.34 (m, 4H), 4.33 (m, 1H), 3.54 (m, 2H), 3.18 (m, 2H), 1.58 (m, 6H), 1.04 (d, J=6.6 Hz, 3H).

### Example 18

### Preparation of (R)-4-Bromo-5-(2-chloro-benzoylamino)-1H-pyrazole-3-carboxylic acid [1-(N,N-dimethylaminocarbonyl)prop-1-yl]amide

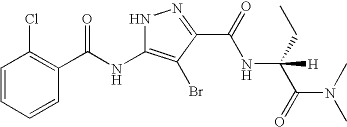

The pyrazole acid, prepared as described in Procedure 5, was coupled with Boc-(R)-(1-dimethylcarbamoyl-propyl)-amine (prepared as described in Procedure 1) using the method of Procedure 2.

MS+=456.0 ¹H-NMR (CDCl₃) δ 9.10 (br, 1H), 8.31 (d, J=8.8 Hz, 1H), 7.99 (d, J=7.1 Hz, 1H), 7.40 (m, 3H), 5.06 (m, 1H), 3.17 (s, 3H), 3.01 (s, 3H), 1.80 (m, 2H), 0.97 (t, J=7.7 Hz, 3H).

### Example 19

### Preparation of (R)-4-Bromo-5-(2-chloro-benzoylamino)-1H-pyrazole-3-carboxylic acid [1-(N-ethyl-N-methylaminocarbonyl)eth-1-yl]amide

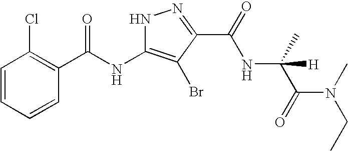

The pyrazole acid, prepared as described in Procedure 5, was coupled with Boc-(R)-[1-(ethyl-methyl-carbamoyl)-ethyl]-amine (prepared as described in Procedure 1) using the method of Procedure 2.

MS+=456.0 ¹H-NMR (CDCl₃) δ 12.87 (br, 1H), 9.23 (s, 1H), 8.21 (m, 1H), 7.95 (m, 1H), 7.41 (m, 3H), 5.07 (m, 1H), 3.45 (m, 2H), 3.08 and 2.95 (two singlets, 3H), 1.40 (t, J=6.0 Hz, 3H), 1.25 and 1.11 (two triplets, J=6.0 Hz, 3H).

### Example 20

### Preparation of (R)-4-Bromo-5-(2-chloro-benzoylamino)-1H-pyrazole-3-carboxylic acid [1-(N,N-diethylaminocarbonyl)eth-1-yl]amide

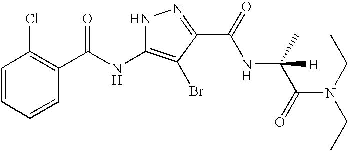

The pyrazole acid, prepared as described in Procedure 5, was coupled with Boc-(R)-(1-diethylcarbamoyl-ethyl)-amine (prepared as described in Procedure 1) using the method of Procedure 2.

MS+=470.0 ¹H-NMR (CDCl₃) δ 12.96 (br, 1H), 9.32 (s, 1H), 8.16 (m, 1H), 7.90 (m, 1H), 7.37 (m, 3H), 5.04 (m, 1H), 3.37 (m, 4H), 1.37 (d, J=6.0 Hz, 3H), 1.22 and 1.10 (two triplets, J=6.0 Hz, 6H).

### Example 21

### Preparation of 4-Bromo-5-(2-chloro-benzoylamino)-1H-pyrazole-3-carboxylic acid [N-methyl-N-phenyl-aminocarbonyl)methyl]amide

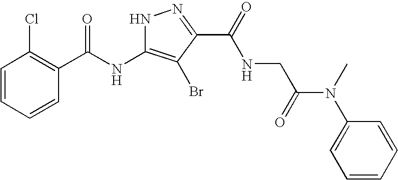

The pyrazole acid, prepared as described in Procedure 5, was coupled with Boc-[(methyl-phenyl-carbamoyl)-methyl]-amine (prepared as described in WO 2002020530) using the method of Procedure 2.

MS+=490.0 ¹H-NMR (CD₃OD) δ 7.54 (m, 9H), 3.92 (s, 2H), 3.34 (s, 3H).

### Example 22

### Preparation of 4-Bromo-5-(2-chloro-benzoylamino)-1H-pyrazole-3-carboxylic acid [(1-methyl-piperizin-4-ylcarbonyl)methyl]amide

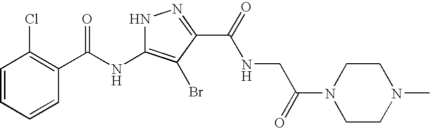

The pyrazole acid, prepared as described in Procedure 5, was coupled with Boc-[2-(4-methyl-piperazin-1-yl)-2-oxo-ethyl]-amine (prepared as described in Procedure 1) using the method described in Procedure 2.

MS+=483.0 ¹H-NMR (DMSO-d6) δ 8.04 (m, 1H), 7.52 (m, 4H), 4.13 (s, 2H), 3.41 (m, 4H), 2.28 (m, 4H).

### Example 23

### Preparation of (R)-4-Bromo-5-(2-chloro-benzoylamino)-1H-pyrazole-3-carboxylic acid [1-(piperidin-1-ylcarbonyl)-3-(phenyl)prop-1-yl]amide

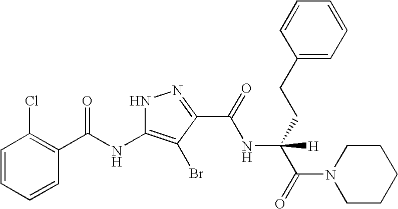

The pyrazole acid, prepared as described in Procedure 5, was coupled with Boc-(R)-[3-phenyl-1-(piperidine-1-carbonyl)-propyl]-amine (prepared as described in Procedure 1) using the method described in Procedure 2.

MS+=572.0 ¹H-NMR (DMSO-d6) δ 13.97 (br, 1H), 7.55 (m, 4H), 7.25 (m, 5H), 4.92 (m, 1H), 3.40 (m, 4H), 2.65 (m, 2H), 1.95 (m, 2H), 1.57 (m, 2H), 1.45 (m, 4H).

### Example 24

### Preparation of [4-Bromo-5-(2-chloro-benzoylamino)-1H-pyrazole-3-carboxylic acid [1-(methoxycarbonyl)-2-(4-hydroxyphen-1-yl)eth-1-yl]amide

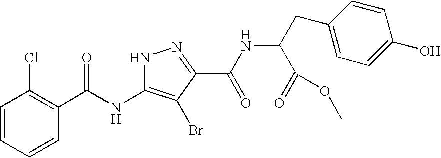

The pyrazole acid, prepared as described in Procedure 5, was coupled with D,L-tyrosine methyl ester hydrochloride (Sigma, T9130) using the method of Procedure 10.

MS+=521.0

### Example 25

### Preparation of 4-Bromo-5-(2-chloro-benzoylamino)-1H-pyrazole-3-carboxylic acid [1-(methoxycarbonyl)-1-(4-hydroxyphen-1-yl)methyl]amide

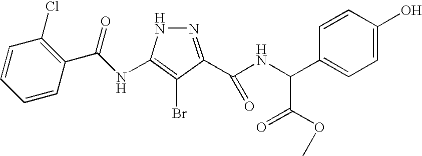

The pyrazole acid, prepared as described in Procedure 5, was coupled with p-hydroxyphenylglycine methyl ester (Aldrich catalog no. 53,492-7) using the method described in Procedure 10.

MS+506.8

### Example 26

### Preparation of (R)-4-Bromo-5-(2-chloro-benzoylamino)-1H-pyrazole-3-carboxylic acid [1-(aminocarbonyl)-2-(methyl)prop-1-yl]amide

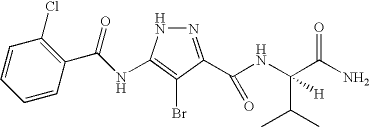

The pyrazole acid was prepared by the methods of Procedure 5 and was coupled with D-valine amide hydrochloride, (NOVABIOCHEM, 04-13-5048) using the method described in Procedure 10.

MS+=441.8

### Example 27

### Preparation of (S)-4-Bromo-5-(2-chloro-benzoylamino)-1H-pyrazole-3-carboxylic acid [1-(aminocarbonyl)eth-1-yl]amide

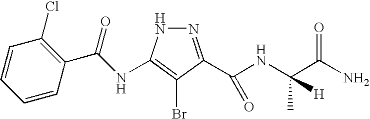

The pyrazole acid, prepared as described in Procedure 5, was coupled with L-alaninamide hydrochloride (Aldrich, 45,921-6) using the method described in Procedure 10.

MS+=413.9

### Example 28

### Preparation of (S)-4-Bromo-5-(2-chloro-benzoylamino)-1H-pyrazole-3-carboxylic acid [1-(phenyl)-1-(aminocarbonyl)methyl]amide

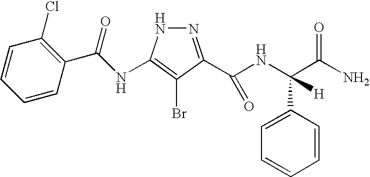

The pyrazole acid, prepared as described in Procedure 5, was coupled with L-phenylglycine amide hydrochloride (Bachem, F-3835) using the method described in Procedure 10.

MS+=475.9

### Example 29

### Preparation of (R)-2-{[4-Bromo-5-(2-chloro-benzoylamino)-1H-pyrazole-3-carboxylic acid [1-(methoxycarbonyl)-2-(1H-indol-3-yl)eth-1-yl]amide

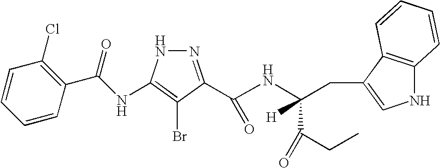

The pyrazole acid, prepared as described in Procedure 5, was coupled with D-tryptophan methyl ester hydrochloride (Aldrich, 36,450-9) using the method described in Procedure 10.

MS+=544.0

### Example 30

### Preparation of 4-Bromo-3-(2-chloro-benzoylamino)-1H-pyrazole-5-arboxylic acid [1-(methoxycarbonyl)-3-methylbut-1-yl]amide

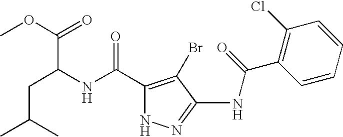

The pyrazole acid, prepared as described in Procedure 5, was coupled with D,L-leucine methyl ester hydrochloride (Bachem, F-2925) using the method described in Procedure 10.

MS+=471.0

### Example 31

### Preparation of 4-Bromo-3-(2-chloro-benzoylamino)-1H-pyrazole-5-carboxylic acid [1-(methoxycarbonyl)eth-1-yl]amide

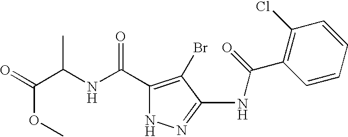

The pyrazole acid, prepared as described in Procedure 5, was coupled with D,L-alanine methyl ester hydrochloride (Bachem, F-1150) using the method described in Procedure 10.

MS+=428.9

### Example 32

### Preparation of (R)-4-Chloro-5-(2-chloro-benzoylamino)-1H-pyrazole-3-carboxylic acid [1-(piperidin-1-ylcarbonyl)eth-1-yl]amide

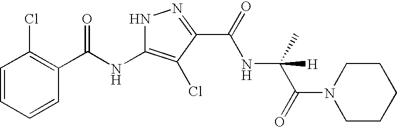

The pyrazole acid, prepared as described in Procedure 5, was coupled with Boc-1-[(2R)-2-amino-1-oxopropyl]piperidine (prepared by Procedure 1) using the method of Procedure 2.

MS+438.0 ¹H-NMR (CDCl₃) δ 9.44 (s, 1H), 8.17 (d, J=7.1 Hz, 1H), 7.81 (d, J=7.1 Hz, 1H), 7.36 (m, 3H), 4.96 (m, 1H), 3.3.51 (m, 4H), 1.56 (m, 6H), 1.30 (d, J=7.0 Hz, 3H).

### Example 33

### Preparation of 4-bromo-5-(2-chlorobenzoylamino)-1H-pyrazole-3-carboxylic acid [(2-(dimethylaminocarbonyl)phen-1-ylaminocarbonyl)methyl]amide

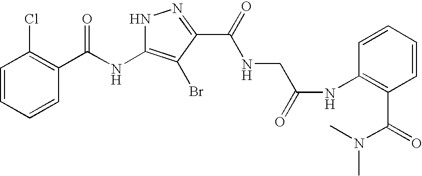

N-tert-Butoxycarbonylglycine (Aldrich, 13,453-8) was coupled to 2-amino-N,N-dimethylbenzamide (Ambinter, 0117380096) using the method described in Procedure 1 to afford [(2-Dimethylcarbamoyl-phenylcarbamoyl)methyl]carbamic acid tert-butyl ester. This was then coupled to the pyrazole acid, prepared as described in Procedure 5, using the method of Procedure 2.

MS+546.9 ¹H-NMR (DMSO-d6) δ 13.99 (br, 1H), 10.79 (br, 1H), 9.48 (br, 1H), 8.56 (br, 1H), 7.85 (m, 1H), 7.51 (m, 5H), 7.30 (m, 1H), 7.22 (m, 1H), 4.02 (m, 2H), 2.52 (s, 6H).

### BIOLOGICAL EXAMPLE

The potency and efficacy to inhibit the bradykinin B₁ receptor was determined for the compounds of this invention in a cell-based fluorescent calcium-mobilization assay. The assay measures the ability of test compounds to inhibit bradykinin B₁ receptor agonist-induced increase of intracellular free Ca⁺² in a native human bradykinin B₁ receptor-expressing cell line.

In this example, the following additional abbreviations have the meanings set forth below. Abbreviations heretofore defined are as defined previously. Undefined abbreviations have the art recognized meanings.
BSA=bovine serum albumin
DMSO=dimethylsulfoxide
FBS=fetal bovine serum
MEM=minimum essential medium
mM=millimolar
ng=nanogram
μg=micrograms
μM=micromolar

Specifically, calcium indicator-loaded cells are pre-incubated in the absence or presence of different concentrations of test compounds followed by stimulation with selective bradykinin B₁ receptor agonist peptide while Ca-dependent fluorescence is monitored.

IMR-90 human lung fibroblast cells (CCL 186, American Type Tissue Collection) are grown in MEM supplemented with 10% FBS as recommended by ATCC. Confluent cells are harvested by trypsinization and seeded into black wall/clear bottom 96-well plates (Costar #3904) at approximately 13,000 cells/well. The following day, cells are treated with 0.35 ng/mL interleukin-1β in 10% FBS/MEM for 2 hours to up-regulate bradykinin B₁ receptors. Induced cells are loaded with fluorescent calcium indicator by incubation with 2.3 μM Fluo-4/AM (Molecular Probes) at 371 C for 1.5 hrs in the presence of an anion transport inhibitor (2.5 mM probenecid in 1% FBS/MEM). Extracellular dye is removed by washing with assay buffer (2.5 mM probenecid, 0.1% BSA, 20 mM HEPES in Hank's Balanced Salt Solution without bicarbonate or phenol red, pH 7.5) and cell plates are kept in dark until used. Test compounds are assayed at 7 concentrations in triplicate wells. Serial dilutions are made in half log-steps at 100-times final concentration in DMSO and then diluted in assay buffer. Compound addition plates contain 2.5-times final concentrations of test compounds or controls in 2.5% DMSO/assay buffer. Agonist plates contain 5-times the final concentration of 2.5 nM (3×EC50) bradykinin B₁ receptor agonist peptide des-Arg¹⁰-kallidin (DAKD, Bachem) in assay buffer. Addition of test compounds to cell plate, incubation for 5 min at 351 C, followed by the addition of bradykinin B₁ receptor agonist DAKD is carried out in the Fluorometric Imaging Plate Reader (FLIPR, Molecular Devices) while continuously monitoring Ca-dependent fluorescence. Peak height of DAKD-induced fluorescence is plotted as function of concentration of test compounds. IC₅₀ values are calculated by fitting a 4-parameter logistic function to the concentration-response data using non-linear regression (Xlfit, IDBS (ID Business Solutions Ltd.)).

Typical potencies observed for bradykinin B₁ receptor agonist peptides are EC₅₀ approximately 0.8 nM and approximately 100 nM for des-Arg¹⁰-kallidin and des-Arg⁹-bradykinin, respectively, while for bradykinin B₁ receptor antagonist peptide des-Arg¹⁰, Leu⁹-kallidin IC₅₀ is approximately 1 nM.

The compounds of this invention have potency in the above assay as demonstrated by results of less than 50 micromolar. It is advantageous that the assay results be less than 1 micromolar, even more advantageous for the results to be less than 0.5 micromolar.

In view of the above, all of these compounds exhibit bradykinin B₁ receptor antagonistic properties and, accordingly, are useful in treating disease conditions mediated at least in part by bradykinin B₁ receptor.

What is claimed is:

1. A compound of Formula (I) or Formula (II): 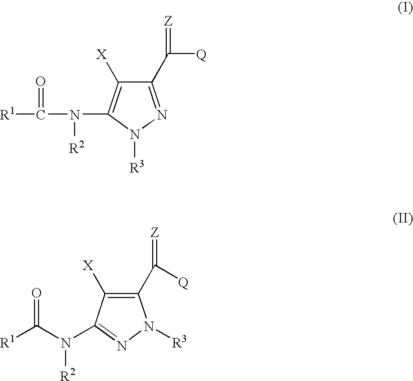
wherein
Z is selected from O, S and NH;
Q is 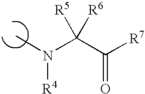
R¹ is selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic and substituted heterocyclic;
R² and R⁴ are independently selected from the group consisting of hydrogen, alkyl and substituted alkyl;
R³ is selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic and substituted heterocyclic;
R⁵ and R⁶ are independently selected from hydrogen, and the side chain of a natural or unnatural amino acid, wherein R⁵ and R⁶ may optionally be linked together to form a cycloalkyl or substituted cycloalkyl;
R⁷ is selected from the group consisting of —NR^{b}R^{c} and —OR^{b} wherein R^{b} and R^{c} are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic, or R^{b} and R^{c} are joined together with the nitrogen atom pendent thereto to form a heterocyclic or substituted heterocyclic group;
X is selected from the group consisting of hydrogen, alkyl, substituted alkyl, alkoxy, substituted alkoxy, aryl, substituted aryl, carboxyl, carboxyl esters, cyano, halo, heteroaryl, substituted heteroaryl, hydroxy, nitro, amino, substituted amino, acylamino, and aminoacyl;
or pharmaceutically acceptable salts, prodrugs or isomers thereof.

2. The compound according to claim 1, wherein Z is O.

3. The compound according to claim 2, wherein R¹ is selected from the group consisting of aryl and substituted aryl.

4. The compound according to claim 3 wherein R¹ is selected from the group consisting of phenyl, naphth-2-yl, naphth-1-yl, monosubstituted phenyls, monosubstituted naphthyls, disubstituted phenyls and trisubstituted phenyls.

5. The compound according to claim 4, wherein R¹ is selected from the group consisting of 5-dimethylaminonaphth-1-yl, 2-chlorophenyl, 2-fluorophenyl, 2-bromophenyl, 2-hydroxyphenyl, 2-nitrophenyl, 2-methylphenyl, 2-methoxyphenyl, 2-phenoxyphenyl, 2-trifluoromethylphenyl, 4-fluorophenyl, 4-chlorophenyl, 4-bromophenyl, 4-nitrophenyl, 4-methylphenyl, 4-hydroxyphenyl, 4-methoxyphenyl, 4-ethoxyphenyl, 4-butoxyphenyl, 4-isopropylphenyl, 4-phenoxyphenyl, 4-trifluoromethylphenyl, 4-hydroxymethylphenyl, 3-methoxyphenyl, 3-hydroxyphenyl, 3-nitrophenyl, 3-fluorophenyl, 3-chlorophenyl, 3-bromophenyl, 3-phenoxyphenyl, 3-thiomethoxyphenyl, 3-methylphenyl, 3-trifluoromethylphenyl, 2,3-dichlorophenyl, 2,3-difluorophenyl, 2,4-dichlorophenyl, 2,5-dimethoxyphenyl, 3,4-dichlorophenyl, 3,4-difluorophenyl, 3,4-methylenedioxyphenyl, 3,4-dimethoxy-phenyl, 3,5-difluorophenyl, 3,5-dichlorophenyl, 3,5-di-(trifluoromethyl)phenyl, 3,5-dimethoxyphenyl, 2,4-dichlorophenyl, 2,4-difluorophenyl, 2,6-difluorophenyl, 3,4,5-trifluorophenyl, 3,4,5-trimethoxyphenyl, 3,4,5-tri-(trifluoromethyl)phenyl, 2,4,6-trifluorophenyl, 2,4,6-trimethylphenyl, 2,4,6-tri-(trifluoromethyl)phenyl, 2,3,5-trifluorophenyl, 2,4,5-trifluorophenyl, 2,5-difluorophenyl, 2-fluoro-3-trifluoromethylphenyl, 4-fluoro-2-trifluoromethylphenyl, 2-fluoro-4-trifluoromethylphenyl, 4-benzyloxyphenyl, 2-chloro-6-fluorophenyl, 2,3,4,5,6-pentafluorophenyl, 2,5-dimethylphenyl, 4-phenylphenyl, 2-fluoro-3-trifluoromethylphenyl, 2-(quinolin-8-yl) thiomethyl)phenyl and 2-((3-methylphen-1-ylthio)methyl)phenyl.

6. The compound according to claim 1, wherein R¹ is selected from the group consisting of alkyl, substituted alkyl, alkenyl and cycloalkyl.

7. The compound according to claim 6, wherein R¹ is selected from the group consisting of isopropyl, n-propyl, n-butyl, isobutyl, sec-butyl, t-butyl, —CH₂CH═CH₂, —CH₂CH═CH(CH₂)₄CH₃, cyclopropyl, cyclobutyl, cyclohexyl, cyclopentyl, —CH₂-cyclopropyl, —CH₂-cyclobutyl, —CH₂-cyclohexyl, —CH₂-cyclopentyl, —CH₂CH₂-cyclopropyl, —CH₂CH₂-cyclobutyl, —CH₂CH₂-cyclohexyl, —CH₂CH₂-cyclopentyl, benzyl, 2-phenyleth-1-yl, and 3-phenyl-n-prop-1-yl.

8. The compound according to claim 1, wherein R¹ is selected from the group consisting of heteroaryl and substituted heteroaryl.

9. The compound according to claim 8, wherein R¹ is selected from the group consisting of pyrid-2-yl, pyrid-3-yl, pyrid-4-yl, 5-fluoropyrid-3-yl, 5-chloropyrid-3-yl, thiophen-2-yl, thiophen-3-yl, benzothiazol-4-yl, 2-phenylbenzoxazol-5-yl, furan-2-yl, benzofuran-2-yl, thionaphthen-2-yl, 2-chlorothiophen-5-yl, 3-methylisoxazol-5-yl, 2-(thiophenyl)thiophen-5-yl, 6-methoxythionaphthen-2-yl, 3-phenyl-1,2,4-thiooxadiazol-5-yl, 2-phenyloxazol-4-yl, 5-chloro-1,3-dimethylpyrazol-4-yl; 2-methoxycarbonyl-thiophen-3-yl; 2,3-dimethylimidazol-5-yl; 2-methylcarbonylamino-4-methyl-thiazol-5-yl; quinolin-8-yl; thiophen-2-yl; 1-methylimidiazol-4-yl; and 3,5-dimethylisoxazol-4-yl.

10. The compound according to claim 1, wherein R¹ is selected from the group consisting of 2-chlorophenyl, 2-fluorophenyl, 2-(quinolin-8-yl) thiomethyl)phen-1-yl and 2-((3-methylphen-1-ylthio)methyl)phen-1-yl.

11. The compound according to claim 1, wherein R¹ is represented by the formula: 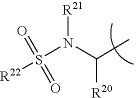
wherein R²¹ is hydrogen or alkyl, and R²⁰ is an amino acid side chain or where R²⁰ and R²¹ and the atoms to which they are attached form a heterocyclic or heteroaryl group of from 4 to 12 ring atoms, and R²² is alkyl, substituted alkyl, aryl or substituted aryl.

12. The compound according to claim 11, wherein R¹ is selected from the group consisting of N-(4-methylbenzenesulfonyl)pyrrol-2-yl, N-(4-chloro-2,5-dimethylbenzenesulfonyl)pyrrol-2-yl, N-(napthylsulfonyl)pyrrol-2-yl, N-(bezylsulfonyl)pyrrol-2-yl; N-(4-chloro-2,5-dimethylbenzenesulfonyl)azetidin-2-yl, N-(4-chloro-2,5-dimethylbenzenesulfonyl)piperidin-2-yl, 1-(4-chloro-2,5-dimethylbenzenesulfonyl)-1,2,3,4-tetrahydroisoquinolin-2-yl, N-(4-chloro-2,5-dimethylbenzenesulfonyl)-N-methyl-aminomethyl; and 1-[N-(4-chloro-2,5-dimethylbenzenesulfonyl)amino]eth-1-yl.

13. The compound according to claim 11, wherein R²² is selected from the group consisting of phenyl, 4-methylphenyl, 2,5-dimethylphenyl, 4-chlorophenyl, 2,5-dimethyl-4-chlorophenyl, benzyl, naphthyl, and 1,2,3,4-tetrahydroisoquinoline.

14. The compound according to claim 13, wherein R²⁰ is hydrogen.

15. The compound according to claim 14, wherein R²¹ is selected from the group consisting of hydrogen, methyl, and ethyl.

16. The compound according to claim 11, wherein R²⁰ and R²¹ are joined to form a heterocyclic group, such as azetidinyl, pyrrolidinyl, piperidinyl, 1,2,3,4-tetrahydroisoquinolinyl, and the like.

17. The compound according to claim 1, wherein R² and R⁴ are independently selected from the group consisting hydrogen, methyl, ethyl, isopropyl, 2-methoxyeth-1-yl, pyrid-3-ylmethyl, benzyl, and t-butoxycarbonyl-methyl.

18. The compound according to claim 17, wherein R³ is selected from the group consisting hydrogen, C₁₋₄alkyl, optionally substituted monocyclic aryl, and optionally substituted monocyclic heteroaryl.

19. The compound according to claim 1, wherein the R⁵ and R⁶ amino acid side chain groups are independently selected from the group consisting of hydrogen, alkyl, substituted alkyl, alkenyl, substituted alkenyl, cycloalkyl, substituted cycloalkyl, cycloalkenyl, substituted cycloalkenyl, aryl, substituted aryl, heteroaryl, substituted heteroaryl, heterocyclic and substituted heterocyclic.

20. The compound according to claim 19, wherein R⁵ is selected from the group consisting of hydrogen, methyl, ethyl, isopropyl, n-propyl, n-butyl, isobutyl, sec-butyl, t-butyl, —CH₂CH₂CH₂NHC(═NH)NH₂, —CH₂C(O)NH₂, —CH₂C(O)OH, —CH₂SH, —CH₂CH₂C(O)OH, —CH₂CH₂C(O)NH₂, imidazol-5-ylmethyl, —CH₂CH₂CH₂CH₂NH₂, —CH₂CH₂SCH₃, hydroxymethyl, 1-hydroxyethyl, phenyl, 4-hydroxyphenyl, benzyl, 4-hydroxybenzyl, 2-phenylethyl, 3-phenyl-n-propyl, 1H-indol-3-ylmethyl, —CH₂CH═CH₂, —CH₂CH═CH(CH₂)₄CH₃, cyclopropyl, cyclobutyl, cyclohexyl, cyclopentyl, cyclohex-1-enyl, —CH₂-cyclopropyl, —CH₂-cyclobutyl, —CH₂-cyclohexyl, —CH₂-cyclopentyl, —CH₂CH₂-cyclopropyl, —CH₂CH₂-cyclobutyl, —CH₂CH₂-cyclohexyl, and —CH₂CH₂-cyclopentyl.

21. The compound according to claim 1, wherein R⁷ is —OR^{b} and R^{b} is selected from the group consisting of hydrogen, methyl, ethyl, isopropyl, and benzyl.

22. The compound according to claim 1, wherein R⁷ is —NR^{a}R^{b} where R^{a} and R^{b} are independently selected from the group consisting of hydrogen, methyl, ethyl, isopropyl, phenyl, benzyl, and 2-dimethylcarbamoylphenyl.

23. The compound according to claim 1, wherein R⁷ is —NR^{a}R^{b} and R^{a} and R^{b} together with the nitrogen atom pendent thereto form a piperidinyl, morpholino, thiomorpholino, pyrazinyl, and 4-methylpyrazin-1-yl group.

24. The compound according to claim 1, wherein Q is selected from the group consisting of ((1-methyl-piperizin-4-ylcarbonyl)methyl)amino; ((N-methyl-N-phenyl-aminocarbonyl)methyl)amino; (1-(R or S)-1-(methoxycarbonyl)-1-(4-hydroxyphen-1-yl)methyl)amino; (1-(R or S)-1-(methoxycarbonyl)-2-(4-hydroxyphen-1-yl)eth-1-yl)amino; (1-(R or S)-1-(methoxycarbonyl)-3-(methyl)but-1-yl)amino; (1-(R or S)-1-(methoxycarbonyl)eth-1-yl)amino; (1-(R or S)-1-(N-morpholinocarbonyl)eth-1-yl)amino; (1-(R)-1-(aminocarbonyl)-2-methylprop-1-yl)amino; (1-(R)-1-(methoxycarbonyl)-2-(1H-indol-3-yl)eth-1-yl)amino; (1-(R)-1-(N,N-dimethylaminocarbonyl)eth-1-yl)amino; (1-(R)-1-(N,N-diethylaminocarbonyl)eth-1-yl)amino; (1-(R)-1-(N,N-dimethylaminocarbonyl)prop-1-yl)amino; (1-(R)-1-(N-ethyl-N-methylaminocarbonyl)eth-1-yl)amino; (1-(R)-1-(N-morpholinocarbonyl)eth-1-yl)amino; (1-(R)-1-(phenyl)-1-(methoxycarbonyl)methyl)amino; (1-(R)-1-(phenyl)-1-(piperidin-1-ylcarbonyl)methyl)amino; (1-(R)-1-(piperidin-1-ylcarbonyl)-3-(phenyl)prop-1-yl)amino; (1-(R)-1-(piperidin-1-ylcarbonyl)eth-1-yl)amino; (1-(S)-1-(aminocarbonyl)eth-1-yl)amino; (1-(S)-1-(methoxycarbonyl)pent-1-yl)amino; (1-(S)-1-(N,N-dimethylaminocarbonyl)eth-1-yl)amino; (1-(S)-1-(phenyl)-1-(aminocarbonyl)methyl)amino; (1-(S)-1-(phenyl)-1-(carboxy)methylamino; (1-(S)-1-(phenyl)-1-(methoxycarbonyl)methyl)amino; (1-(S)-1-(phenyl)-1-(methylaminocarbonyl) methyl)amino; (1-(S)-1-(phenyl)-1-(piperidin-1-ylcarbonyl)methyl)amino; (1-(S)-1-(piperidin-1-ylcarbonyl)eth-1-yl)amino; (1-(S)-2-(phenyl)-1-(methoxycarbonyl)eth-1-yl)amino; and [(2-(dimethylaminocarbonyl)phen-1-ylaminocarbonyl)methyl]amino.

25. The compound according to claim 1, wherein X is selected from the group consisting of hydrogen, bromine, chlorine and methyl.

26. A compound selected from the group consisting of:
(R)4-bromo-5-(2-chloro-benzoylamino)-1H-pyrazole-3-carboxylic acid (1-(N,N-dimethylaminocarbonyl)eth-1-yl)amide;
(S)-4-bromo-5-(2-chloro-benzoylamino)-1H-pyrazole-3-carboxylic acid (1-(N,N-dimethylaminocarbonyl)eth-1-yl)amide;
(S)-4-bromo-5-(2-chloro-benzoylamino)-1H-pyrazole-3-carboxylic acid [1-(phenyl)-1-(methoxycarbonyl)methyl]amide;
(S)-4-bromo-5-(2-chloro-benzoylamino)-1H-pyrazole-3-carboxylic acid [1-(phenyl)-1-(carboxy)methyl]amide;
(S)-5-(2-chloro-benzoylamino)-1H-pyrazole-3-carboxylic acid [1-(phenyl)-1-(methoxycarbonyl)methyl]amide;
(S)-4-bromo-5-(2-chloro-benzoylamino)-1H-pyrazole-3-carboxylic acid [2-(phenyl)-1-(methoxycarbonyl)eth-1-yl)]amide;
(R)-4-bromo-5-(2-chloro-benzoylamino)-1H-pyrazole-3-carboxylic acid [1-(phenyl)-1-(methoxycarbonyl)methyl]amide;
(S)-4-bromo-5-(2-m-tolylthiomethyl-benzoylamino)-1H-pyrazole-3-carboxylic acid [1-(phenyl)-1-(methoxycarbonyl)methyl]amide;
(S)-2-{[4-bromo-5-(2-chloro-benzoylamino)-1H-pyrazole-3-carboxylic acid [1-(methoxycarbonyl)pent-1-yl]amide;
(S)-4-bromo-5-(2-chloro-benzoylamino)-1H-pyrazole-3-carboxylic acid [1-(phenyl)-1-(methylaminocarbonyl)methyl]amide;
(S)-4-bromo-5-(2-chloro-benzoylamino)-1H-pyrazole-3-carboxylic acid [1-(piperidin-1-ylcarbonyl)eth-1-yl]amide;
4-bromo-5-(2-chloro-benzoylamino)-1H-pyrazole-3-carboxylic acid [1-(N-morpholinocarbonyl)eth-1-yl]amide;
(R)-4-bromo-5-(2-chloro-benzoylamino)-1H-pyrazole-3-carboxylic acid [1-(piperidin-1-ylcarbonyl)eth-1-yl]amide;
(S)-4-bromo-5-(2-chloro-benzoylamino)-1H-pyrazole-3-carboxylic acid [1-(phenyl)-1-(piperidin-1-ylcarbonyl)methyl]amide;
(R)-4-bromo-5-(2-chloro-benzoylamino)-1H-pyrazole-3-carboxylic acid [1-(N-morpholinocarbonyl)eth-1-yl]amide;
(R)-4-bromo-5-(2-chloro-benzoylamino)-1H-pyrazole-3-carboxylic acid [1-(phenyl)-1-(piperidin-1-ylcarbonyl)methyl]amide;
(R)-5-(2-chloro-benzoylamino)-1H-pyrazole-3-carboxylic acid [1-(piperidin-1-ylcarbonyl)eth-1-yl]amide;
(R)-4-bromo-5-(2-chloro-benzoylamino)-1H-pyrazole-3-carboxylic acid [1-(N,N-dimethylaminocarbonyl)prop-1-yl]amide;
(R)-4-bromo-5-(2-chloro-benzoylamino)-1H-pyrazole-3-carboxylic acid [1-(N-ethyl-N-methylaminocarbonyl)eth-1-yl]amide;
(R)-4-bromo-5-(2-chloro-benzoylamino)-1H-pyrazole-3-carboxylic acid [1-(N,N-diethylaminocarbonyl)eth-1-yl]amide;
4-bromo-5-(2-chloro-benzoylamino)-1H-pyrazole-3-carboxylic acid [N-methyl-N-phenyl-aminocarbonyl)methyl]amide;
4-bromo-5-(2-chloro-benzoylamino)-1H-pyrazole-3-carboxylic acid [(1-methyl-piperizin-4-ylcarbonyl)methyl]amide;
(R)-4-bromo-5-(2-chloro-benzoylamino)-1H-pyrazole-3-carboxylic acid [1-(piperidin-1-ylcarbonyl)-3-(phenyl)prop-1-yl]amide;
4-bromo-5-(2-chloro-benzoylamino)-1H-pyrazole-3-carboxylic acid [1-(methoxycarbonyl)-2-(4-hydroxyphen-1-yl)eth-1-yl]amide;
4-bromo-5-(2-chloro-benzoylamino)-1H-pyrazole-3-carboxylic acid [1-(methoxycarbonyl)-1-(4-hydroxyphen-1-yl)methyl]amide;
(R)-4-bromo-5-(2-chloro-benzoylamino)-1H-pyrazole-3-carboxylic acid [1-(aminocarbonyl)-2-(methyl)prop-1-yl]amide;
(S)-4-bromo-5-(2-chloro-benzoylamino)-1H-pyrazole-3-carboxylic acid [1-(aminocarbonyl)eth-1-yl]amide;
(S)-4-bromo-5-(2-chloro-benzoylamino)-1H-pyrazole-3-carboxylic acid [1-(phenyl)-1-(aminocarbonyl)methyl]amide;
(R)-4-bromo-5-(2-chloro-benzoylamino)-1H-pyrazole-3-carboxylic acid [1-(methoxycarbonyl)-2-(1H-indol-3-yl)eth-1-yl]amide;
4-bromo-3-(2-chloro-benzoylamino)-1H-pyrazole-5-carboxylic acid [1-(methoxycarbonyl)-3-methylbut-1-yl]amide;
4-bromo-3-(2-chloro-benzoylamino)-1H-pyrazole-5-carboxylic acid [1-(methoxycarbonyl)eth-1-yl]amide;
(R)-4-chloro-5-(2-chloro-benzoylamino)-1H-pyrazole-3-carboxylic acid [1-(piperidin-1-ylcarbonyl)eth-1-yl]amide;
4-bromo-5-(2-chlorobenzoylamino)-1H-pyrazole-3-carboxylic acid [(2-(dimethylaminocarbonyl)phen-1-ylaminocarbonyl)methyl]amide; or
pharmaceutically acceptable salts thereof.

27. A method for selectively inhibiting bradykinin B₁ receptor relative to the bradykinin B₂ receptor which method comprises contacting an inhibiting effective amount of a compound of claim 1 to a biological sample comprising both the bradykinin B₁ and B₂ receptors.

28. A pharmaceutical composition comprising a pharmaceutically acceptable carrier and an amount of a compound of claim 1 or mixtures thereof effective to treat or palliate adverse symptoms in mammals mediated at least in part by the bradykinin B₁ receptor.

29. A method for treating or palliating adverse symptoms in a mammal mediated at least in part by bradykinin B₁ receptor which method comprises administering a therapeutically effective amount of a compound of claim 1 or mixtures thereof.

30. A method for treating or palliating adverse symptoms in a mammal mediated at least in part by bradykinin B₁ receptor which method comprises administering to said mammal a therapeutically effective amount of a pharmaceutical composition of claim 28.

31. A method for treating or palliating adverse symptoms in a mammal associated with up-regulation of the bradykinin B₁ receptor following tissue damage or inflammation which method comprises administering to said mammal a therapeutically effective amount of a compound of claim 1 or mixtures thereof.

32. A method for treating or palliating adverse symptoms in a mammal associated with up-regulation of the bradykinin B₁ receptor following tissue damage or inflammation which method comprises administering to said mammal a therapeutically effective amount of the pharmaceutical composition of claim 28.

33. A method for treating or palliating adverse symptoms associated with the presence or secretion of bradykinin B₁ receptor agonists in a mammal which method comprises administering a therapeutically effective amount of a compound of claim 1 or mixtures thereof.

34. A method for treating or palliating adverse symptoms associated with the presence or secretion of bradykinin B₁ receptor agonists in a mammal which method comprises administering a therapeutically effective amount of the pharmaceutical composition of claim 28.

35. A method for treating or ameliorating pain, inflammation, septic shock or the scarring process in mammals mediated at least in part by bradykinin B₁ receptor which method comprises administering a therapeutically effective amount of a compound of claim 1 or mixtures thereof.

36. A method for treating or ameliorating pain, inflammation, septic shock or the scarring process in mammals mediated at least in part by bradykinin B₁ receptor which method comprises administering a therapeutically effective amount of a pharmaceutical composition according to claim 28.

37. A method for treating or ameliorating adverse symptoms in a mammal associated with up-regulating bradykinin B₁ receptor relative to burns, perioperative pain, migraine, shock, central nervous system injury, asthma, rhinitis, premature labor, inflammatory arthritis, inflammatory bowel disease or neuropathic pain which method comprises administering a therapeutically effective amount of a compound of claim 1 or mixtures thereof.

38. A method for treating or ameliorating adverse symptoms in a mammal associated with up-regulating bradykinin B₁ receptor relative to burns, perioperative pain, migraine, shock, central nervous system injury, asthma, rhinitis, premature labor, inflammatory arthritis, inflammatory bowel disease or neuropathic pain which method comprises administering a therapeutically effective amount of a pharmaceutical composition according to claim 28.

39. A method for treating or palliating adverse symptoms associated with the presence or secretion of bradykinin B₁ receptor agonists in mammals which method comprises administering a therapeutically effective amount of a compound of claim 1 or mixtures thereof.

40. A method for treating or palliating adverse symptoms associated with the presence or secretion of bradykinin B₁ receptor agonists in mammals which method comprises administering a therapeutically effective amount of a pharmaceutical composition according to claim 28.
